(19) United States
(12) Patent Application Publication
(10) Pub. No.: US 2010/0184640 A1
(43) Pub. Date: Jul. 22, 2010

(54) DOG DIABETES

(75) Inventors: Christopher Andrew Jones, Nottingham (GB); Neale Fretwell, Nottingham (GB); Brian Catchpole, Hertfordshire (GB); Lorna Jane Kennedy, Manchester (GB); William Ernest Royce Ollier, Manchester (GB)

Correspondence Address: FULBRIGHT & JAWORSKI, LLP, 1301 MCKINNEY, SUITE 5100, HOUSTON, TX, 77010-3095, US

(73) Assignee: MARS INCORPORATED, McLEAN, VA (US)

(21) Appl. No.: 12/095,622

(22) PCT Filed: Nov. 30, 2006
(86) PCT No.: PCT/GB2006/004476

§ 371 Date: Feb. 19, 2009

(30) Foreign Application Priority Data
Nov. 30, 2005 (GB) 0524421.5

(51) Int. Cl. A61K 38/28; C12Q 1/68; C07H 21/04; G01N 33/68; A61P 3/10; A23K 1/18; G06F 19/00

(52) U.S. Cl. 514/3; 435/6; 536/24.31; 536/24.33; 435/7.21; 426/2; 702/19

(57) ABSTRACT

A method a method for diagnosing susceptibility to diabetes in a non-human animal, the method comprising: a) identifying whether or not a polymorphism as defined in Table 4 or a polymorphism which is in linkage disequilibrium with such a polymorphism is present in the genome of the animal; and b) thereby diagnosing whether the animal is susceptible to diabetes, wherein optionally the said identifying is carried out on a sample from the animal.

### FIELD OF THE INVENTION

The present invention relates to the diagnosis and treatment of diabetes in animals.

### BACKGROUND OF THE INVENTION

Use of an assay which identifies animals that are susceptible to diabetes would then allow such animals to be given therapy for diabetes.

### SUMMARY OF THE INVENTION

The present inventors have identified polymorphism markers in animals which are associated with diabetes.

Accordingly, the invention provides a method for diagnosing susceptibility to diabetes in a non-human animal, the method comprising:

a) identifying whether or not a polymorphism as defined in Table 4 or a polymorphism which is in linkage disequilibrium with such a polymorphism is present in the genome of the animal; and

b) thereby diagnosing whether the animal is susceptible to diabetes,

wherein optionally the said identifying is carried out on a sample from the animal.

The invention further provides:
a probe, primer or antibody which is capable of detecting the polymorphisms;
a kit for carrying out the method of the invention comprising means for detecting the polymorphisms;
a method of preparing customised food for an animal which is susceptible to diabetes, the method comprising:

(a) determining whether the animal is susceptible to diabetes by a method of the invention; and

(b) preparing food suitable for the animal;
a database comprising information relating to polymorphisms and optionally their association with diabetes.

### DESCRIPTION OF THE FIGURES

FIG. 1 shows odds ratio and confidence interval for protective and risk haplotypes in all dogs.

FIG. 2 shows percentage of dogs by risk group with DLA-DRB1*009 haplotypes.

FIG. 3 shows percentage of dogs by risk group with DLA-DQA1*004/DQB1*013 haplotypes.

FIG. 4 shows percentage of dogs by risk group with DLA-DQA1 alleles containing Arg 55.

FIG. 5 shows an apparatus of the invention.

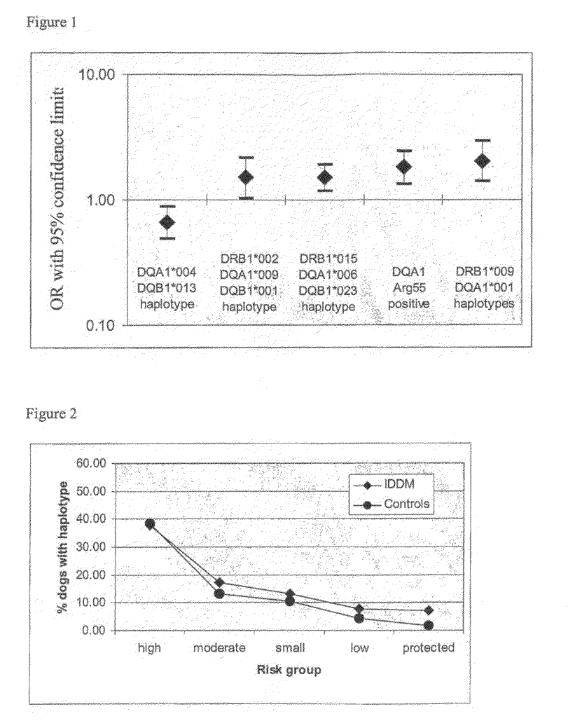

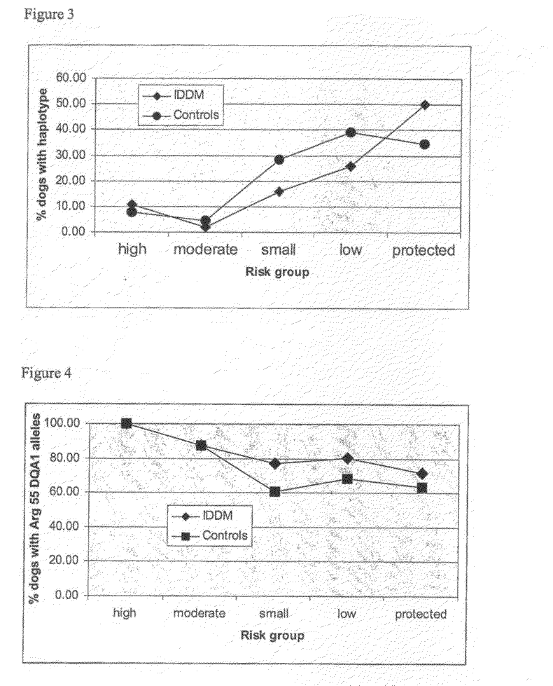

### DETAILED DESCRIPTION OF THE INVENTION

The present invention provides a method for determining susceptibility to diabetes in an animal. The diabetes condition is normally one which is caused, at least partially, by an autoimmune mechanism.

The animal tested is typically a mammal, preferably a non-human animal, such as a dog, cat, horse, pig, cattle or sheep. The animal may be a companion animal or pet. In a preferred embodiment, the animal tested is a dog. The dog tested may be of any breed, or may be a mixed or crossbred dog, or an outbred dog (mongrel). The dog may be of any of the breeds mentioned herein.

The animal may be from 0 to 10 years old, for example from 0 to 5 years old, from 0 to 3 years old or from 0 to 2 years old. When the method of the invention is carried out on a sample from the animal, the sample may have been taken from an animal within any of these age ranges. The animal may be tested by the method of the invention before any symptoms of diabetes are apparent.

A dog of any breed may be tested by a method of the present invention. The table below provides examples of dog breeds, wherein S=small, M=medium, L=large and XL=extra large.

| | |
|---|---|
| Breed | Size |

a) Hounds

| | |
|---|---|
| Afghan Hound | L |
| Basenji | M |
| Basset Bleu De Gascogne | M |
| Basset Fauve De Bretagne | M |
| Basset Griffon Vendeen (Grand) | M |
| Basset Griffon Vendeen (Petit) | M |
| Basset Hound | M |
| Bavarian Mountain Hound | M |
| Beagle | M |
| Bloodhound | L |
| Borzoi | L |
| Dachshund | M |
| Dachshund (Long Haired) | M |
| Dachshund (Miniature Long Haired) | S |
| Dachshund (Short Haired) | M |
| Dachshund (Smooth Haired) | M |
| Dachshund (Miniature Smooth Haired) | S |
| Dachshund (Wire Haired) | M |
| Dachshund (Miniature Wire Haired) | S |
| Deerhound | L |
| Norwegian Elkhound | L |
| Finnish Spitz | M |
| Foxhound | L |
| Grand Bleu De Gascogne | L |
| Greyhound | L |
| Hamiltonstovare | L |
| Ibizan Hound | L |
| Irish Wolfhound | XL |
| Norwegian Lundehund | M |
| Otterhound | L |
| Pharaoh Hound | L |
| Rhodesian Ridgeback | L |
| Saluki | L |
| Segugio Italiano | L |
| Sloughi | L |
| Whippet | M |

b) Working Dogs

| | |
|---|---|
| Alaskan Malamute | L |
| Beauceron | L |
| Bernese Mountain Dog | XL |
| Bouvier Des Flandres | L |
| Boxer | L |
| Bullmastiff | L |
| Canadian Eskimo Dog | L |
| Dobermann | L |
| Dogue de Bordeaux | L |
| German Pinscher | M |
| Greenland Dog | L |
| Giant Schnauzer | L |
| Great Dane | XL |
| Hovawart | L |
| Leonberger | XL |
| Mastiff | XL |
| Neapolitan Mastiff | XL |
| Newfoundland | XL |
| Portuguese Water Dog | L |
| Rottweiler | L |
| Russian Black Terrier | L |
| St. Bernard | XL |
| Siberian Husky | L |
| Tibetan Mastiff | XL |

c) Terrier

| | |
|---|---|
| Airedale Terrier | L |
| Australian Terrier | S |
| Bedlington Terrier | M |
| Border Terrier | S |
| Bull Terrier | M |
| Bull Terrier (Miniature) | M |
| Cairn Terrier | S |
| Cesky Terrier | M |
| Dandie Dinmont Terrier | M |
| Fox Terrier (Smooth) | M |
| Fox Terrier (Wire) | M |
| Glen of Imaal Terrier | M |
| Irish Terrier | M |
| Jack Russell Terrier | M |
| Kerry Blue Terrier | M |
| Lakeland Terrier | M |
| Manchester Terrier | M |
| Norfolk Terrier | S |
| Norwich Terrier | S |
| Parson Russell Terrier | M |
| Scottish Terrier | M |
| Sealyham Terrier | M |
| Skye Terrier | M |
| Soft Coated Wheaten Terrier | M |
| Staffordshire Bull Terrier | M |
| Welsh Terrier | M |
| West Highland White Terrier | S |

d) Gundogs (Sporting Group)

| | |
|---|---|
| Bracco Italiano | L |
| Brittany | M |
| English Setter | L |
| German Longhaired Pointer | L |
| German Shorthaired Pointer | L |
| German Wirehaired Pointer | L |
| Gordon Setter | L |
| Hungarian Vizsla | L |
| Hungarian Wirehaired Vizsla | L |
| Irish Red and White Setter | L |
| Irish Setter | L |
| Italian Spinone | L |
| Kooikerhondje | M |
| Lagotto Romagnolo | M |
| Large Munsterlander | L |
| Nova Scotia Duck Tolling Retriever | M |
| Pointer | L |
| Retriever (Chesapeake Bay) | L |
| Retriever (Curly Coated) | L |
| Retriever (Flat Coated) | L |
| Retriever (Golden) | L |
| Retriever (Labrador) | L |
| Spaniel (American Cocker) | M |
| Spaniel (American Water) | M |
| Spaniel (Clumber) | L |
| Spaniel (Cocker) | M |
| Spaniel (English Cocker) | M |
| Spaniel (English Springer) | M |
| Spaniel (Field) | M |
| Spaniel (Irish Water) | M |
| Spaniel (Sussex) | M |
| Spaniel (Welsh Springer) | M |
| Spanish Water Dog | M |
| Vizsla | M |
| Weimaraner | L |

e) Pastoral (Herding Group)

| | |
|---|---|
| Anatolian Shepherd Dog | L |
| Australian Cattle Dog | M |
| Australian Shepherd | L |
| Bearded Collie | L |
| Belgian Shepherd Dog (Groenendael) | L |
| Belgian Shepherd Dog (Malinois) | L |
| Belgian Shepherd Dog (Laekenois) | L |
| Belgian Shepherd Dog (Tervueren) | L |
| Bergamasco | L |
| Border Collie | M |
| Briard | L |
| Collie (Rough) | L |
| Collie (Smooth) | L |
| Estrela Mountain Dog | XL |
| Finnish Lapphund | M |
| German Shepherd Dog (Alsatian) | L |
| Hungarian Kuvasz | L |
| Hungarian Puli | M |
| Komondor | L |
| Lancashire Heeler | S |
| Maremma Sheepdog | L |
| Norwegian Buhund | M |
| Old English Sheepdog | L |
| Polish Lowland Sheepdog | M |
| Pyrenean Mountain Dog | XL |
| Pyrenean Sheepdog | M |
| Samoyed | L |
| Shetland Sheepdog | M |
| Swedish Lapphund | M |
| Swedish Vallhund | M |
| Welsh Corgi (Cardigan) | M |
| Welsh Corgi (Pembroke) | M |

f) Utility Dogs (Non-sporting)

| | |
|---|---|
| Akita | L |
| American Eskimo | M |
| Boston Terrier | S |
| Bulldog | M |
| Canaan Dog | L |
| Chow Chow | L |
| Dalmatian | L |
| French Bulldog | S |
| German Spitz (Klein) | S |
| German Spitz (Mittel) | M |
| Japanese Shiba Inu | M |
| Japanese Spitz | M |
| Keeshond | M |
| Lhasa Apso | S |
| Mexican Hairless | M |
| Miniature Schnauzer | S |
| Poodle (Miniature) | M |
| Poodle (Standard) | L |
| Poodle (Toy) | S |
| Schipperke | S |
| Schnauzer (Standard) | M |
| Shar Pei | M |
| Shih Tzu | S |
| Tibetan Spaniel | S |
| Tibetan Terrier | M |

g) Toy Dogs

| | |
|---|---|
| Affenpinscher | S |
| Australian Silky Terrier | S |
| Bichon Frise | S |
| Bolognese | S |
| Cavalier King Charles Spaniel | S |
| Chihuahua (Long Coat) | S |
| Chihuahua (Smooth Coat) | S |
| Chinese Crested | S |
| Coton De Tulear | S |
| English Toy Terrier (Black and Tan) | S |
| Griffon Bruxellios | S |
| Havanese | S |
| Italian Greyhound | S |
| Japanese Chin | S |
| King Charles Spaniel | S |
| Lowchen (Little Lion Dog) | S |
| Maltese | S |
| Miniature Pinscher | S |
| Papillon | S |
| Pekingese | S |
| Pomeranian | S |
| Pug | S |
| Silky Terrier | S |
| Toy Fox Terrier | S |
| Yorkshire Terrier | S |

In a preferred embodiment the dog to be tested is a dog which is of a breed mentioned in Table 1 or Table 3. In particular the dog may be of any of the following breeds: Samoyed, Tibetan Terrier, Bichon Frise, Yorkshire Terrier, Schnauzer (miniature), Border Collie, Dachshund, Border Terrier or Poodle; or a dog that is genetically related to any of these breeds. Preferably the dog to be tested is a pure bred. However, in one embodiment, the dog to be tested may have at least 50% of any of the breeds mentioned herein. In another embodiment, the dog may have at least 75% of any of the breeds mentioned herein in its genetic bred background. Thus, at least 50% or at least 75% of its genome may be derived from any of the breeds mentioned herein. In one embodiment, the dog may have a parent or grandparent which is of any of the breeds mentioned herein. The genetic breed background of a dog may be determined by detecting the presence or absence of two or more breed-specific SNP markers in the dog.

### Detection of Polymorphisms

The detection of polymorphisms according to the invention may comprise contacting a polynucleotide or protein of the animal with a specific binding agent for a polymorphism and determining whether the agent binds to the polynucleotide or protein, wherein binding of the agent indicates the presence of the polymorphism, and lack of binding of the agent indicates the absence of the polymorphism.

The method is generally carried out in vitro on a sample from the animal. The sample typically comprises a body fluid and/or cells of the individual and may, for example, be obtained using a swab, such as a mouth swab. The sample may be a blood, urine, saliva, skin, cheek cell or hair root sample. The sample is typically processed before the method is carried out, for example DNA extraction may be carried out. The polynucleotide or protein in the sample may be cleaved either physically or chemically, for example using a suitable enzyme. In one embodiment the part of polynucleotide in the sample is copied or amplified, for example by cloning or using a PCR based method prior to detecting the polymorphism.

In the present invention, any one or more methods may comprise determining the presence or absence of one or more polymorphisms in the animal. The polymorphism is typically detected by directly determining the presence of the polymorphic sequence in a polynucleotide or protein of the animal. Such a polynucleotide is typically genomic DNA, mRNA or cDNA. The polymorphism may be detected by any suitable method such as those mentioned below.

A specific binding agent is an agent that binds with preferential or high affinity to the protein or polypeptide having the polymorphism but does not bind or binds with only low affinity to other polypeptides or proteins. The specific binding agent may be a probe or primer. The probe may be a protein (such as an antibody) or an oligonucleotide. The probe may be labelled or may be capable of being labelled indirectly. The binding of the probe to the polynucleotide or protein may be used to immobilise either the probe or the polynucleotide or protein.

Generally in the method, determination of the binding of the agent to the polymorphism can be carried out by determining the binding of the agent to the polynucleotide or protein of the animal. However in one embodiment the agent is also able to bind the corresponding wild-type sequence, for example by binding the nucleotides or amino acids which flank the polymorphism position, although the manner of binding to the wild-type sequence will be detectably different to the binding of a polynucleotide or protein containing the polymorphism.

The method may be based on an oligonucleotide ligation assay in which two oligonucleotide probes are used. These probes bind to adjacent areas on the polynucleotide which contains the polymorphism, allowing after binding the two probes to be ligated together by an appropriate ligase enzyme. However the presence of single mismatch within one of the probes may disrupt binding and ligation. Thus ligated probes will only occur with a polynucleotide that contains the polymorphism, and therefore the detection of the ligated product may be used to determine the presence of the polymorphism.

In one embodiment the probe is used in a heteroduplex analysis based system. In such a system when the probe is bound to polynucleotide sequence containing the polymorphism it forms a heteroduplex at the site where the polymorphism occurs and hence does not form a double strand structure. Such a heteroduplex structure can be detected by the use of single or double strand specific enzyme. Typically the probe is an RNA probe, the heteroduplex region is cleaved using RNAase H and the polymorphism is detected by detecting the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment a PCR primer is used that primes a PCR reaction only if it binds a polynucleotide containing the polymorphism, for example a sequence- or allele-specific PCR system, and the presence of the polymorphism may be determined by the detecting the PCR product. Preferably the region of the primer which is complementary to the polymorphism is at or near the 3′ end of the primer. The presence of the polymorphism may be determined using a fluorescent dye and quenching agent-based PCR assay such as the Taqman PCR detection system. The specific binding agent may be capable of specifically binding the amino acid sequence encoded by a variant sequence. For example, the agent may be an antibody or antibody fragment. The detection method may be based on an ELISA system. The method may be an RFLP based system. This can be used if the presence of the polymorphism in the polynucleotide creates or destroys a restriction site that is recognised by a restriction enzyme.

The presence of the polymorphism may be determined based on the change which the presence of the polymorphism makes to the mobility of the polynucleotide or protein during gel electrophoresis. In the case of a polynucleotide single-stranded conformation polymorphism (SSCP) or denaturing gradient gel electrophoresis (DDGE) analysis may be used.

The presence of the polymorphism may be detected by means of fluorescence resonance energy transfer (FRET). In particular, the polymorphism may be detected by means of a dual hybridisation probe system. This method involves the use of two oligonucleotide probes that are located close to each other and that are complementary to an internal segment of a target polynucleotide of interest, where each of the two probes is labelled with a fluorophore. Any suitable fluorescent label or dye may be used as the fluorophore, such that the emission wavelength of the fluorophore on one probe (the donor) overlaps the excitation wavelength of the fluorophore on the second probe (the acceptor). A typical donor fluorophore is fluorescein (FAM), and typical acceptor fluorophores include Texas red, rhodamine, LC-640, LC-705 and cyanine 5 (Cy5).

In order for fluorescence resonance energy transfer to take place, the two fluorophores need to come into close proximity on hybridisation of both probes to the target. When the donor fluorophore is excited with an appropriate wavelength of light, the emission spectrum energy is transferred to the fluorophore on the acceptor probe resulting in its fluorescence. Therefore, detection of this wavelength of light, during excitation at the wavelength appropriate for the donor fluorophore, indicates hybridisation and close association of the fluorophores on the two probes. Each probe may be labelled with a fluorophore at one end such that the probe located upstream (5′) is labelled at its 3′ end, and the probe located downstream (3′) is labelled at is 5′ end. The gap between the two probes when bound to the target sequence may be from 1 to 20 nucleotides, preferably from 1 to 17 nucleotides, more preferably from 1 to 10 nucleotides, such as a gap of 1, 2, 4, 6, 8 or 10 nucleotides.

The first of the two probes may be designed to bind to a conserved sequence of the gene adjacent to a polymorphism and the second probe may be designed to bind to a region including one or more polymorphisms. Polymorphisms within the sequence of the gene targeted by the second probe can be detected by measuring the change in melting temperature caused by the resulting base mismatches. The extent of the change in the melting temperature will be dependent on the number and base types involved in the nucleotide polymorphisms.

The polymorphic position may be typed directly, in other words by determining the nucleotide present at that position, or indirectly, for example by determining the nucleotide present at another polymorphic position that is in linkage disequilibrium with said polymorphic position.

Polymorphisms which are in linkage disequilibrium with each other in a population are typically found together on the same chromosome. Typically one is found at least 30% of the times, for example at least 40%, at least 50%, at least 70% or at least 90%, of the time the other is found on a particular chromosome in individuals in the population. Thus a polymorphism which is not a functional susceptibility polymorphism, but is in linkage disequilibrium with a functional polymorphism, may act as a marker indicating the presence of the functional polymorphism.

Polymorphisms which are in linkage disequilibrium with the polymorphisms mentioned herein are typically located within 500 kb, preferably within 400 kb, within 200 kb, within 100 kb, within 50 kb, within 10 kb, within 5 kb, within 1 kb, within 500 bp, within 100 bp, within 50 bp or within 10 bp of the polymorphism.

A polynucleotide of the invention may be used as a primer, for example for PCR, or a probe. A polynucleotide or polypeptide of the invention may carry a revealing label. Suitable labels include radioisotopes such as ³²P or ³⁵S, fluorescent labels, enzyme labels or other protein labels such as biotin.

Polynucleotides of the invention may be used as a probe or primer which is capable of selectively binding to a polymorphism. The invention thus provides a probe or primer for use in a method according to the invention, which probe or primer is capable of selectively detecting the presence of a polymorphism. Preferably the probe is isolated or recombinant nucleic acid. The probe may be immobilised on an array, such as a polynucleotide array.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of a full length polynucleotide sequence of the invention.

Polypeptides of the invention may be chemically modified, for example post-translationally modified. The polypeptides may be glycosylated or comprise modified amino acid residues. Such modified polypeptides fall within the scope of the term “polypeptide” of the invention.

The polypeptides (e.g. antibodies) and polynucleotides (e.g. primer and probes) of the invention may be present in an isolated or substantially purified form. They may be mixed with carriers or diluents which will not interfere with their intended use and still be regarded as substantially isolated. They may also be in a substantially purified form, in which case they will generally comprise at least 90%, e.g. at least 95%, 98% or 99%, of the proteins or polynucleotides or dry mass of the preparation.

In the method of the invention the presence or absence of the alleles mentioned in Table 4 may be detected by any suitable means. Typically in the method one or more of the polymorphisms listed in Table 4 is typed. Thus, the presence or absence of the polymorphism may be determined, typically in a polynucleotide from the dog, to ascertain whether or not the genome of the dog comprises the relevant polymorphism. In one embodiment, whether or not the genome of the dog comprises all of the polymorphisms listed a row of Table 4 is acertained. Thus for example, the method may comprise determining the presence or absence of 96C, 126A and 254G as shown in the top row of polymorphisms in Table 4. In a preferred embodiment, at least 5, at least 15 or at least 20 of the polymorphisms shown in Table 4 are typed in the method of the invention. In one embodiment, a polymorphism which is in linkage to disequilibrium with a polymorphism shown in Table 4 is typed (in order to acesertain the presence of a polymorphism in Table 4 in the genome of the dog). In one embodiment, whether or not the polymorphisms which are typed are present on the same DNA strand is also determined.

### Detector Antibodies

The invention also provides detector antibodies that are specific for a polypeptide of the invention. A detector antibody is specific for one polymorphism, for example. The detector antibodies of the invention are for example useful in purification, isolation or screening methods involving immunoprecipitation techniques.

Antibodies may be raised against specific epitopes of the polypeptides of the invention. An antibody, or other compound, “specifically binds” to a polypeptide when it binds with preferential or high affinity to the protein for which it is specific but does substantially bind not bind or binds with only low affinity to other polypeptides. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample (such as any such sample mentioned herein), which method comprises:
I providing an antibody of the invention;
II incubating a biological sample with said antibody under conditions which allow for the formation of an antibody-antigen complex; and
III determining whether antibody-antigen complex comprising said antibody is formed.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”. The fragment may be any of the fragments mentioned herein (typically at least 10 or at least 15 amino acids long).

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified. A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

### Detection Kit

The invention also provides a kit that comprises means for determining the presence or absence of one or more polymorphisms in an animal which are associated with susceptibility to diabetes. In particular, such means may include a specific binding agent, probe, primer, pair or combination of primers, or antibody, including an antibody fragment, as defined herein which is capable of detecting or aiding detection of a polymorphism. The primer or pair or combination of primers may be sequence specific primers which only cause PCR amplification of a polynucleotide sequence comprising the polymorphism to be detected, as discussed herein. The kit may also comprise a specific binding agent, probe, primer, pair or combination of primers, or antibody which is capable of detecting the absence of the polymorphism. The kit may further comprise buffers or aqueous solutions.

The kit may additionally comprise one or more other reagents or instruments which enable any of the embodiments of the method mentioned above to be carried out. Such reagents or instruments may include one or more of the following: a means to detect the binding of the agent to the polymorphism, a detectable label such as a fluorescent label, an enzyme able to act on a polynucleotide, typically a polymerase, restriction enzyme, ligase, RNAse H or an enzyme which can attach a label to a polynucleotide, suitable buffer(s) or aqueous solutions for enzyme reagents, PCR primers which bind to regions flanking the polymorphism as discussed herein, a positive and/or negative control, a gel electrophoresis apparatus, a means to isolate DNA from sample, a means to obtain a sample from the individual, such as swab or an instrument comprising a needle, or a support comprising wells on which detection reactions can be carried out. The kit may be, or include, an array such as a polynucleotide array comprising the specific binding agent, preferably a probe, of the invention. The kit typically includes a set of instructions for using the kit.

### Screening for Therapeutic Agents

The present invention also relates to the use of polypeptides encoded by the polymorphic sequence as a screening target for identifying therapeutic agents for the treatment of diabetes. In one embodiment the invention provides a method for identifying an agent useful for the treatment of diabetes, which method comprises contacting the polypeptide with a test agent and determining whether the agent is capable of binding to the polypeptide or modulating the activity or expression of the polypeptide. Any suitable binding assay format can be used to determine whether the polypeptide binds the test agent, such as the formats discussed below.

The method may be carried out in vitro, either inside or outside a cell, or in vivo. In one embodiment the method is carried out on a cell, cell culture or cell extract that comprises the polypeptide.

The method may also be carried out in vivo in an non-human animal which is transgenic for a polymorphism as defined herein. The transgenic non-human animal is typically of a species commonly used in biomedical research and is preferably a laboratory strain. Suitable animals include rodents, particularly a mouse, rat, guinea pig, ferret, gerbil or hamster. Most preferably the animal is a mouse.

Suitable candidate agents which may be tested in the above screening methods include antibody agents, for example monoclonal and polyclonal antibodies, single chain antibodies, chimeric antibodies and CDR-grafted antibodies. Furthermore, combinatorial libraries, defined chemical identities, peptide and peptide mimetics, oligonucleotides and natural agent libraries, such as display libraries may also be tested. The test agents may be chemical compounds, which are typically derived from synthesis around small molecules which may have any of the properties of the agent mentioned herein. Batches of the candidate agents may be used in an initial screen of, for example, ten substances per reaction, and the substances of batches which show modulation tested individually. The term ‘agent’ is intended to include a single substance and a combination of two, three or more substances. For example, the term agent may refer to a single peptide, a mixture of two or more peptides or a mixture of a peptide and a defined chemical entity. In one aspect of the invention, the test agent is a food ingredient.

### Treatment of Diabetes

The invention provides a method of treating an animal for diabetes. The method comprising identifying an animal which is susceptible to diabetes by the above-described method, and administering to the animal an effective amount of a therapeutic agent which treats diabetes. The therapeutic agent may be any drug known in the art that may be used to treat diabetes, or may an agent identified by a screening method as discussed previously.

The therapeutic agent may be administered in various manners such as orally, intracranially, intravenously, intramuscularly, intraperitoneally, intranasally, intrademally, and subcutaneously. The pharmaceutical compositions that contain the therapeutic agent will normally be formulated with an appropriate pharmaceutically acceptable carrier or diluent depending upon the particular mode of administration being used. For instance, parenteral formulations are usually injectable fluids that use pharmaceutically and physiologically acceptable fluids such as physiological saline, balanced salt solutions, or the like as a vehicle. Oral formulations, on the other hand, may be solids, for example tablets or capsules, or liquid solutions or suspensions.

The amount of therapeutic agent that is given to an animal will depend upon a variety of factors including the condition being treated, the nature of the animal under treatment and the severity of the condition under treatment. A typical daily dose is from about 0.1 to 50 mg per kg, preferably from about 0.1 mg/kg to 10 mg/kg of body weight, according to the activity of the specific inhibitor, the age, weight and conditions of the animal to be treated, the type and severity of the disease and the frequency and route of administration. Preferably, daily dosage levels are from 5 mg to 2 g.

### Customised Food

In one aspect, the invention relates to a customised diet for an animal that is susceptible to diabetes. In a preferred embodiment, the customised food is for a companion animal or pet, such as a dog. Such a food may be in the form of, for example, wet pet foods, semi-moist pet foods, dry pet foods and pet treats. Wet pet food generally has a moisture content above 65%. Semi-moist pet food typically has a moisture content between 20-65% and can include humectants and other ingredients to prevent microbial growth. Dry pet food, also called kibble, generally has a moisture content below 20% and its processing typically includes extruding, drying and/or baking in heat. The ingredients of a dry pet food generally include cereal, grains, meats, poultry, fats, vitamins and minerals. The ingredients are typically mixed and put through an extruder/cooker. The product is then typically shaped and dried, and after drying, flavours and fats may be coated or sprayed onto the dry product.

Accordingly, the present invention enables the preparation of customised food suitable for an animal which is susceptible to diabetes, wherein the customised animal food formulation comprises ingredients that prevent or alleviate diabetes (for example, in an increased amount), and/or does not comprise components that contribute to or aggravate diabetes or comprises components that contribute to or aggravate diabetes in a reduced amount. Such ingredients may be any of those known in the art to prevent or alleviate diabetes, such as insulin. Alternatively, screening methods as discussed herein may identify such ingredients. The preparation of customised animal food may be carried out by electronic means, for example by using a computer system.

In another embodiment, the customised food may be formulated to include functional or active ingredients that help prevent or alleviate diabetes.

The present invention also relates to a method of providing a customised animal food, comprising providing food suitable for an animal which is susceptible to diabetes to the animal, the animal's owner or the person responsible for feeding the animal, wherein the animal has been determined to be susceptible to diabetes by a method of the invention. In one aspect of the invention, the customised food is made to inventory and supplied from inventory, i.e. the customised food is pre-manufactured rather than being made to order. Therefore according this aspect of the invention the customised food is not specifically designed for one particular animal but instead is suitable for more than one animal. For example, the customised food may be suitable for any animal that is susceptible to diabetes. Alternatively, the customised food may be suitable for a sub-group of animals that are susceptible to diabetes, such as animals of a particular breed, size or lifestage. In another embodiment, the food may be customised to meet the nutritional requirements of an individual animal.

### Bioinformatics

The sequences of the polymorphisms may be stored in an electronic format, for example in a computer database. Accordingly, the invention provides a database comprising information relating to polymorphismsequences. The database may include further information about the polymorphism, for example the level of association of the polymorphism with diabetes or the frequency of the polymorphism in the population. In one aspect of the invention, the database further comprises information regarding the food components which are suitable and the food components which are not suitable for animals who possess a particular polymorphism.

A database as described herein may be used to determine the susceptibility of an animal to diabetes. Such a determination may be carried out by electronic means, for example by using a computer system (such as a PC). Typically, the determination will be carried out by inputting genetic data from the animal to a computer system; comparing the genetic data to a database comprising information relating to polymorphisms; and on the basis of this comparison, determining the susceptibility of the animal to diabetes.

The invention also provides a computer program comprising program code means for performing all the steps of a method of the invention when said program is run on a computer. Also provided is a computer program product comprising program code means stored on a computer readable medium for performing a method of the invention when said program is run on a computer. A computer program product comprising program code means on a carrier wave that, when executed on a computer system, instruct the computer system to perform a method of the invention is additionally provided.

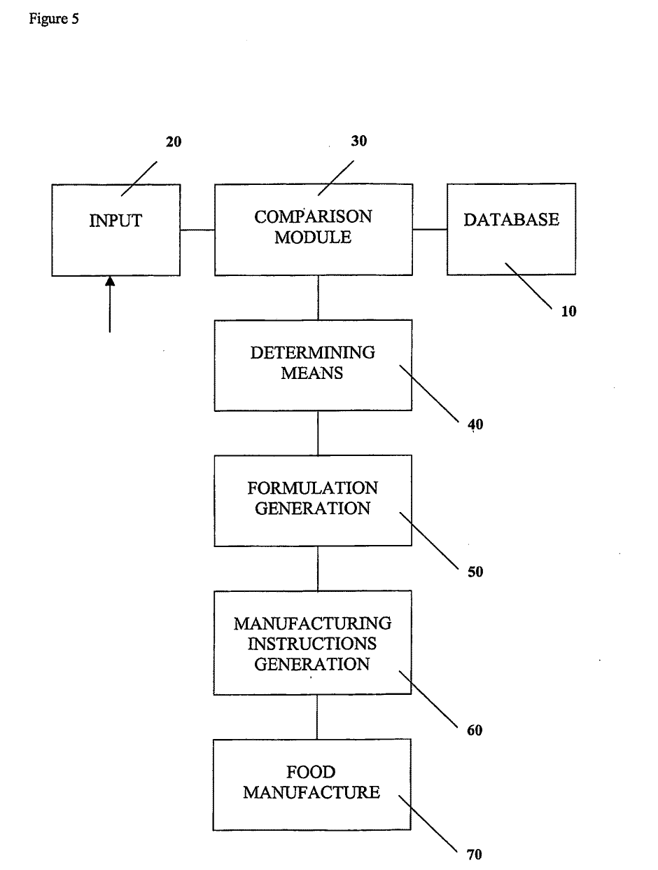

As illustrated in FIG. 5, the invention also provides an apparatus arranged to perform a method according to the invention. The apparatus typically comprises a computer system, such as a PC. In one embodiment, the computer system comprises: means 20 for receiving genetic data from the animal; a module 30 for comparing the data with a database 10 comprising information relating to polymorphisms; and means 40 for determining on the basis of said comparison the susceptibility of the animal to diabetes.

### Food Manufacturing

In one embodiment of the invention, the manufacture of a customised animal food may be controlled electronically. Typically, information relating to the polymorphism present in an animal may be processed electronically to generate a customised animal food formulation. The customised animal food formulation may then be used to generate electronic manufacturing instructions to control the operation of food manufacturing apparatus. The apparatus used to carry out these steps will typically comprise a computer system, such as a PC, which comprises means 50 for processing the nutritional information to generate a customised animal food formulation; means 60 for generating electronic manufacturing instructions to control the operation of food manufacturing apparatus; and a food product manufacturing apparatus 70.

The food product manufacturing apparatus used in the present invention typically comprises one or more of the following components: container for dry pet food ingredients; container for liquids; mixer; former and/or extruder; cut-off device; cooking means (e.g. oven); cooler; packaging means; and labelling means. A dry ingredient container typically has an opening at the bottom. This opening may be covered by a volume-regulating element, such as a rotary lock. The volume-regulating element may be opened and closed according to the electronic manufacturing instructions to regulate the addition of dry ingredients to the pet food.

Dry ingredients typically used in the manufacture of pet food include corn, wheat, meat and/or poultry meal. Liquid ingredients typically used in the manufacture of pet food include fat, tallow and water. A liquid container may contain a pump that can be controlled, for example by the electronic manufacturing instructions, to add a measured amount of liquid to the pet food.

In one embodiment, the dry ingredient container(s) and the liquid container(s) are coupled to a mixer and deliver the specified amounts of dry ingredients and liquids to the mixer. The mixer may be controlled by the electronic manufacturing instructions. For example, the duration or speed of mixing may be controlled. The mixed ingredients are typically then delivered to a former or extruder. The former/extruder may be any former or extruder known in the art that can be used to shape the mixed ingredients into the required shape. Typically, the mixed ingredients are forced through a restricted opening under pressure to form a continuous strand. As the strand is extruded, it may be cut into pieces (kibbles) by a cut-off device, such as a knife. The kibbles are typically cooked, for example in an oven. The cooking time and temperature may be controlled by the electronic manufacturing instructions. The cooking time may be altered in order to produce the desired moisture content for the food. The cooked kibbles may then be transferred to a cooler, for example a chamber containing one or more fans.

The food manufacturing apparatus may comprise a packaging apparatus. The packaging apparatus typically packages the food into a container such as a plastic or paper bag or box. The apparatus may also comprise means for labelling the food, typically after the food has been packaged. The label may provide information such as: ingredient list; nutritional information; date of manufacture; best before date; weight; and species and/or breed(s) for which the food is suitable.

The invention is illustrated by the following Examples:

### EXAMPLES

### Materials and Methods

Control DNA samples were obtained from residual blood samples taken for diagnostic clinical purposes at the Small Animal Hospital, University of Liverpool. Table 1 shows the breed distribution of the 460 diabetics, 1047 controls and 69 female entire diabetics.

All the dogs were characterised for three DLA class II loci using either sequence based typing (SBT) (Kennedy et al Tissue Antigens 60: 43-52, 2002; Kennedy et al Immunogenetics 48: 296-301, 1998) or Reference Strand-mediated Conformation Analysis (RSCA).

All PCR reactions are performed with 25 ng DNA in a 25 μl reaction containing 1×PCR buffer as supplied by Qiagen (with no extra magnesium), Q solution (Qiagen), final concentrations of 0.1 μM for each primer, 200 μM each dNTP, with 2 units of Taq polymerase, (Qiagen HotStarTaq). A negative control containing no DNA template should be included in each run of amplifications to identify any contamination.

Primers used were: DRBF forward: gat ccc ccc gtc ccc aca g, DRBR3 reverse: cgc ccg ctg cgc tca, DQAin1 forward: taa ggt tct ttt etc cct ct, DQAIn2 reverse: gga cag att cag tga aga ga, DQB1B forward: ctc act ggc ccg get gtc tc and DQBR2 reverse: cac etc gcc get gca acg tg. All primers are intronic and locus specific, and the product sizes are 303 bp for DLA-DRB1, 345 bp for DQA1 and 300 bp for DQB1.

A standard Touchdown PCR protocol was used for all amplifications, which consisted of an initial 15 minutes at 95° C., 14 touch down cycles of 95° C. for 30 seconds, followed by 1 minute annealing, starting at 62° C. (DRB1), 54° C. (DQA1) 73° C. (DQB1) and reducing by 0.5° C. each cycle, and 72° C. for 1 minute. Then 20 cycles of 95° C. for 30 seconds, 55° C. (DRB1), 47° C. (DQA1) 66° C. (DQB1) for 1 minute, 72° C. for 1 minute plus a final extension at 72° C. for 10 minutes.

To check for the presence of a product, 5 μl was run on a 2% agarose gel. No purification was required for RSCA. However, this was required SBT: 2 units of shrimp alkaline phosphatase (USB) and 10 units of Exol (New England Biolabs) were added to 5 μl of PCR product. The mixture was incubated for 1 hour at 37° C., then for 15 minutes at 80° C.

RSCA: FLRs were generated, using a range of DLA-DRB1 alleles from the domestic dog and grey wolf. The FLRs were produced by PCR using cloned alleles as templates and a 5′-FAM22 labelled forward primer. In order to increase the proportion of the labelled reference strand in the reaction, the primer proportions were altered to 0.5 μM FAM22-labelled forward primer and 0.1 μM reverse unlabelled primer. All other aspects of the PCR reaction remained the same. This single stranded-biased FLR was used to increase the heights of the FLR-allele heteroduplex peaks relative to the homoduplex peaks in subsequent RSCA. All the resulting FLRs were diluted 1:30 in water before use in the hybridisation reactions.

In order to form duplexes between test samples and FLRs, 2 μl of diluted FLR and 2 μl of test sample PCR product were mixed in a 96 well plate and incubated in a thermal cycler at 95° C. for 10 minutes, ramped down to 55° C. at 1° C./second, 55° C. for 15 minutes and 4° C. for 15 minutes. The plate was stored at 4° C. until required. Subsequently, 8 μl distilled water were added to each hybridisation reaction, and then 2 μl were mixed with 4.8 μl water and 0.2 μl Genescan Rox-500 size standards (Applied Biosystems), in a 384 well plate. These samples were run on an ABI 3100 DNA analyser, using 50 cm capillary arrays, 4% Genescan non-denaturing polymer (Applied Biosystems) and data collected using matrix Dye set D. The conditions were: injection voltage 15 kV, injection time 15 seconds, run voltage 15 kV, run temperature 30° C. Each run took 35 minutes. The data were analysed using software programs “Genescan” and “Genotyper” (Applied Biosystems). Genescan was used to assign sizes to each peak, based on the ROX-500 standards. Using Genotyper, allele peaks formed by the control samples were assigned to “bins” for each FLR used. The bins were exported to a program which assigned the alleles for each sample.

Three-locus, DLA-DRB1/DQA1/DQB1, haplotypes were identified by following a sequential analytical process. Firstly, all dogs that were homozygous at all three loci were selected, and from these several different DLA-DRB1-DQA1-DQB1 haplotype combinations were identified. Dogs that were homozygous at only two loci were then selected. From these dogs many of the previous haplotypes were confirmed and also several further haplotypes were identified. The remaining dogs were examined using the haplotype data already identified and haplotypes were assigned to each of these dogs. From these dogs further possible haplotypes were identified.

TABLE 1
Distribution of dog breeds in the patient and control data sets

| | | | FE |
|---|---|---|---|
| | IDDM | Controls | IDDM |
| Breed | n = 460 | n = 1047 | n = 69 |
| Afghan Hound | | 2 | |
| Australian Shepherd Dog | 1 | 2 | |
| Basset Hound | | 8 | |
| Beagle | 5 | 59 | 2 |
| Bernese Mountain Dog | | 7 | |
| Bichon Frise | 11 | 21 | 3 |
| Bloodhound | | 1 | |
| Bouvier | | 2 | |
| Boxer | | 51 | 1 |
| Briard | | 3 | |
| Bull Mastiff | | 15 | 1 |
| Bulldog | | 3 | |
| Chow Chow | | 6 | 1 |
| Collie (Bearded) | 1 | 3 | 1 |
| Collie (Border) | 26 | 41 | 11 |
| Collie (Rough) | | 5 | |
| Corgi | 4 | 3 | |
| Dachshund (All types) | 11 | 25 | 4 |
| Dalmatian | 1 | 4 | |
| Deerhound | | 1 | |
| Doberman | 5 | 36 | 1 |
| Elkhound | 1 | | |
| Foxhound | | 2 | |
| German Shepherd Dog | | 57 | 1 |
| Great Dane | 1 | 6 | |
| Greyhound | | 2 | |
| Hovawart | | 6 | |
| Husky | 1 | 12 | 1 |
| Irish Wolfhound | | 5 | |
| Japanese Akita | | 4 | |
| Labrador | 56 | 93 | 7 |
| Lhasa Apso | 3 | 4 | |
| Lurcher | 1 | 4 | |
| Mastiff | | 3 | |
| Munsterlander (Large) | | 2 | |
| Newfoundland | 1 | 5 | |
| Papillon | | 7 | |
| Pharaoh Hound | | 1 | |
| Pinscher (Miniature) | 1 | | |
| Pointer | | 4 | |
| Polish lowland sheepdog | 2 | | |
| Pomeranian | 2 | 2 | |
| Poodle (All types) | 8 | 25 | |
| Pug | | 1 | |
| Pyrenean Mountain Dog | | 4 | 1 |
| Retriever (Chesapeake Bay) | 1 | | |
| Retriever (Floatcoat) | | 2 | |
| Retriever (Golden) | 6 | 44 | 1 |
| Rhodesian Ridgeback | 1 | 18 | |
| Rottweiler | 4 | 19 | |
| Samoyed | 15 | 9 | 4 |
| Schnauzer (Miniature) | 10 | 14 | |
| Setter (English) | 3 | 33 | |
| Setter (Gordon) | 3 | 3 | |
| Setter (Irish) | 1 | 9 | |
| Sharpei | | 2 | |
| Sheepdog (Old English) | 3 | 8 | |
| Sheepdog (Shetland) | 4 | 3 | |
| Shih Tzu | 2 | 19 | |
| Spaniel (CKCS) | 19 | 17 | |
| Spaniel (Clumber) | | 1 | |
| Spaniel (Cocker) | 15 | 30 | 2 |
| Spaniel (Field) | | 1 | |
| Spaniel (Springer) | 8 | 21 | 1 |
| Spinone (Italian) | | 2 | |
| Spitz | 1 | 2 | 1 |
| St Bernard | | 5 | |
| Terrier (Airedale) | | 2 | |
| Terrier (Border) | 10 | 11 | |
| Terrier (Boston) | | 1 | |
| Terrier (Bull) | 1 | 2 | |
| Terrier (Cairn) | 15 | 11 | 3 |
| Terrier (Dandie Dinmont) | 1 | | |
| Terrier (Fox) | 1 | 2 | |
| Terrier (Jack Russell) | 17 | 40 | 3 |
| Terrier (Maltese) | | 2 | |
| Terrier (Manchester) | | 1 | |
| Terrier (Patterdale) | | | 1 |
| Terrier (Scottish) | 1 | 3 | |
| Terrier (Staffs Bull) | 3 | 8 | |
| Terrier (Tibetan) | 7 | 6 | |
| Terrier (Welsh) | | 1 | |
| Terrier (West Highland | 38 | 33 | 4 |
| White) | | | |
| Terrier (Yorkshire) | 29 | 47 | 3 |
| Vizsla Hungarian | | 5 | |
| Weimaraner | 1 | 5 | |
| Whippet | 1 | 2 | |
| X-Crossbreed | 97 | 56 | 11 |

TABLE 2
Percentage of IDDM, control and female entire IDDM dogs with each haplotype

| | | | IDDM | | Controls | | FE IDDM | | Odds | |
|---|---|---|---|---|---|---|---|---|---|---|
| DRB1 | DQA1 | DQB1 | n = 460 | % | n = 1047 | % | n = 69 | % | Ratio | P value |
| 001 | 001 | 002 | 96 | 20.87 | 219 | 20.92 | 20 | 28.99 | | |
| 001 | 001 | 036 | 10 | 2.17 | 20 | 1.91 | | 0.00 | | |
| 001 | 003 | 004 | 4 | 0.87 | 17 | 1.62 | | 0.00 | | |
| 001 | 009 | 001 | 10 | 2.17 | 12 | 1.15 | 3 | 4.35 | | |
| 002 | 009 | 001 | 54 | 11.74 | 85 | 8.12 | 9 | 13.04 | 1.51 | 0.03 |
| 004 | 002 | 015 | 1 | 0.22 | 44 | 4.20 | | 0.00 | | |
| 005 | 003 | 005 | 5 | 1.09 | 13 | 1.24 | | 0.00 | | |
| 006 | 004 | 013 | 7 | 1.52 | 39 | 3.72 | 1 | 1.45 | | |
| 006 | 005 | 007 | 81 | 17.61 | 180 | 17.19 | 9 | 13.04 | | |
| 006 | 005 | 02001 | 8 | 1.74 | 24 | 2.29 | 1 | 1.45 | | |
| 008 | 003 | 004 | 10 | 2.17 | 21 | 2.01 | 1 | 1.45 | | |
| 009 | 001 | 008 | 55 | 11.96 | 65 | 6.21 | 4 | 5.80 | 2.05 | 0.0002 |
| 011 | 002 | 013 | 29 | 6.30 | 66 | 6.30 | 5 | 7.25 | | |
| 012 | 001 | 002 | 2 | 0.43 | 14 | 1.34 | 1 | 1.45 | | |
| 012 | 004 | 013 | 8 | 1.74 | 24 | 2.29 | 1 | 1.45 | | |
| 012 | 004 | 013017 | 37 | 8.04 | 105 | 10.03 | 5 | 7.25 | | |
| 013 | 001 | 002 | 40 | 8.70 | 70 | 6.69 | 7 | 10.14 | | |
| 015 | 006 | 003 | 7 | 1.52 | 27 | 2.58 | 2 | 2.90 | | |
| 015 | 006 | 019022 | 11 | 2.39 | 8 | 0.76 | 4 | 5.80 | | |
| 015 | 006 | 02002 | 25 | 5.43 | 37 | 3.53 | 3 | 4.35 | | |
| 015 | 006 | 022 | 13 | 2.83 | 14 | 1.34 | 7 | 10.14 | | |
| 015 | 006 | 023 | 158 | 34.35 | 268 | 25.60 | 15 | 21.74 | 1.52 | 0.0006 |
| 015 | 009 | 001 | 13 | 2.83 | 52 | 4.97 | 2 | 2.90 | | |
| 018 | 001 | 002 | 12 | 2.61 | 21 | 2.01 | 4 | 5.80 | | |
| 018 | 001 | 008 | 4 | 0.87 | 32 | 3.06 | 1 | 1.45 | | |
| 020 | 004 | 013 | 25 | 5.43 | 77 | 7.35 | 3 | 4.35 | | |
| 023 | 003 | 005 | 12 | 2.61 | 13 | 1.24 | 2 | 2.90 | | |
| other | Rare | haplos | 48 | 10.43 | 149 | 14.23 | 9 | 13.04 | | |
| | Arg55 | | 388 | 84.35 | 783 | 74.78 | 59 | 85.5 | 1.82 | .00005 |
| | 004 | 013 | 76 | 16.52 | 242 | 23.11 | 9 | 13.04 | 0.66 | 0.005 |

TABLE 3
Percentage of dogs from selected breeds with a high risk and a protective haplotype

| | | | | | DRB1*009 | | | | | | | |
|---|---|---|---|---|---|---|---|---|---|---|---|---|
| | | | | | haplotypes | | | | DQA1*004/DQB1*013 | | | |
| | Risk | | IDDM | controls | IDDM | | controls | | IDDM | | controls | |
| | Ratio | Breed | n | n | n | % | n | % | n | % | n | % |
| High | 17.30 | Samoyed | 15 | 9 | 8 | 53.33 | 5 | 55.56 | 4 | 26.67 | 2 | 22.22 |
| High | 6.93 | Terrier (Tibetan) | 7 | 6 | 1 | 14.29 | 2 | 33.33 | | | | |
| High | 6.77 | Terrier (Cairn) | 15 | 11 | 5 | 33.33 | 3 | 27.27 | | | | |
| moderate | 3.60 | Bichon Frise | 11 | 21 | | | 3 | 14.29 | | | | |
| moderate | 3.48 | Terrier | 29 | 47 | | | | | | | 2 | 4.26 |
| | | (Yorkshire) | | | | | | | | | | |
| moderate | 3.18 | Schnauzer | 10 | 14 | 7 | 70.00 | 13 | 92.86 | | | | |
| | | (Miniature) | | | | | | | | | | |
| moderate | 2.89 | Collie (Border) | 26 | 41 | 3 | 11.54 | 3 | 7.32 | 2 | 7.69 | 3 | 7.32 |
| moderate | 2.83 | Dachshund (all | 11 | 25 | 7 | 63.64 | 4 | 16.00 | | | 2 | 8.00 |
| | | types) | | | | | | | | | | |
| moderate | 2.51 | Terrier (Border) | 10 | 11 | | | | | | | 1 | 9.09 |
| moderate | 2.40 | Poodle (All types) | 8 | 25 | 1 | 12.50 | 1 | 4.00 | | | | |
| small | 1.74 | Rottweiler | 4 | 19 | | | | | | | | |
| small | 1.70 | Terrier (WHWT) | 38 | 33 | | | | | | | | |
| small | 1.48 | Terrier (Jack | 17 | 40 | 3 | 17.65 | 7 | 17.50 | | | 3 | 7.50 |
| | | Russell) | | | | | | | | | | |
| small | 1.45 | Spaniel (CKCS) | 19 | 17 | 8 | 42.11 | 7 | 41.18 | 8 | 42.11 | 2 | 11.76 |
| small | 1.22 | Doberman | 5 | 36 | | | 1 | 2.78 | 5 | 100.00 | 36 | 100.00 |
| Low | 0.97 | Labrador | 56 | 93 | | | 3 | 3.23 | 25 | 44.64 | 58 | 62.37 |
| Low | 0.78 | X-Crossbreed | 97 | 56 | 13 | 13.40 | 5 | 8.93 | 18 | 18.56 | 11 | 19.64 |
| Low | 0.75 | Spaniel (Cocker) | 15 | 30 | | | | | | | 1 | 3.33 |
| protected | 0.42 | Spaniel (English | 8 | 21 | 1 | 12.50 | 3 | 14.29 | 2 | 25.00 | 12 | 57.14 |
| | | springer) | | | | | | | | | | |
| protected | 1.19 | Retriever | 6 | 44 | | | | | 5 | 83.33 | 37 | 84.09 |
| | | (Golden) | | | | | | | | | | |
| protected | 0.15 | German | | 57 | | | | | | | 8 | 14.04 |
| | | Shepherd dog | | | | | | | | | | |
| protected | 0.07 | Boxer | | 51 | | | | | | | 3 | 5.88 |
| | | All IDDM no FE | 460 | 1047 | 62 | 13.48 | 74 | 7.07 | 76 | 16.52 | 242 | 23.11 |

TABLE 4
Diabetes Susceptibility Alleles and Polymorphism which are typed

| | | | | | | | | |
|---|---|---|---|---|---|---|---|---|
| DRB1*00201 | 98 C | 126 A | 254 G | | | | | |
| DRB1*00901 | 95 A | 185 T | | | | | | |
| DRB1*01501 | 11 G | 62 C | 126 A | 161 G | 173 G | 218 G | 236 G | 254 A |
| DRB1*01502 | 24 T | 62 C | 254 G | | | | | |
| DQB1*00101 | 10 A | 93 C | 152 G | | | | | |
| DQB1*008011 | 22 G | 104 C | 124 A | 173 A | 237 A | | | |
| DQB1*008012 | 104 T | | | | | | | |
| DQB1*00802 | 22 G | 124 T | 173 A | | | | | |
| DQB1*01301 | 22 T | 172 C | 237 A | | | | | |
| DQB1*02301 | 10 A | 22 G | 94 A | 124 A | 154 C | 237 T | | |
| DQA1*00101 | 12 A | 58 A | 148 G | | | | | |
| DQA1*00401 | 84 G | 148 C | 189 A | 210 C | | | | |
| dqa1*00402 | 84 T | 189 A | | | | | | |
| DQA1*00601 | 12 T | 148 G | 189 A | 210 C | | | | |
| DQA1*00901 | 12 T | 58 T | 148 G | | | | | |

| DQB1 | 460 | % | 652 | % | 1124 | % | c | d | d | | X2 | OR | Cl | p |
|---|---|---|---|---|---|---|---|---|---|---|---|---|---|---|
| 001 | 77 | 16.74 | 110 | 16.87 | 168 | 14.95 | 383 | 542 | 956 | | | | | |
| 002 | 152 | 33.04 | 227 | 34.82 | 352 | 31.32 | 308 | 425 | 772 | | | | | |
| 003 | 7 | 1.52 | 15 | 2.30 | 27 | 2.40 | 453 | 637 | 1097 | | | | | |
| 003v | | 0.00 | | 0.00 | 23 | 2.05 | 460 | 652 | 1101 | | | | | |
| 004 | 14 | 3.04 | 29 | 4.45 | 55 | 4.89 | 446 | 623 | 1069 | | | | | |
| 005 | 18 | 3.91 | 21 | 3.22 | 26 | 2.31 | 442 | 631 | 1098 | | | | | |
| 00502 | | 0.00 | | 0.00 | 1 | 0.09 | 460 | 652 | 1123 | | | | | |
| 007 | 82 | 17.83 | 120 | 18.40 | 231 | 20.55 | 378 | 532 | 893 | | | | | |
| 008 | 54 | 11.74 | 50 | 7.67 | 83 | 7.38 | 406 | 602 | 1041 | x | 4.8 | 1.29 | 1.06-1.57 | 0.03 |
| 00802 | 10 | 2.17 | 26 | 3.99 | 78 | 6.94 | 450 | 626 | 1046 | | | | | |
| 011 | 5 | 1.09 | | 0.00 | | 0.00 | 455 | 652 | 1124 | | | | | |
| 013 | 71 | 15.43 | 106 | 16.26 | 218 | 19.40 | 389 | 546 | 906 | x | ns | | | |
| 013017 | 37 | 8.04 | 74 | 11.35 | 108 | 9.61 | 423 | 578 | 1016 | | | | | |
| 015 | 2 | 0.43 | 6 | 0.92 | 46 | 4.09 | 458 | 646 | 1078 | x | 13.64 | 0.1 | 0.02-0.43 | 3E−04 |
| 017 | | 0.00 | 1 | 0.15 | 3 | 0.27 | 460 | 651 | 1121 | | | | | |
| 019 | 2 | 0.43 | 4 | 0.61 | 8 | 0.71 | 458 | 648 | 1116 | | | | | |
| 019022 | 12 | 2.61 | 8 | 1.23 | 8 | 0.71 | 448 | 644 | 1116 | | | | | |
| 02001 | 8 | 1.74 | 22 | 3.37 | 24 | 2.14 | 452 | 630 | 1100 | | | | | |
| 02002 | 25 | 5.43 | 26 | 3.99 | 36 | 3.20 | 435 | 626 | 1088 | | | | | |
| 022 | 13 | 2.83 | 12 | 1.84 | 14 | 1.25 | 447 | 640 | 1110 | | | | | |
| 023 | 159 | 34.57 | 180 | 27.61 | 264 | 23.49 | 301 | 472 | 860 | x | 5.84 | 1.39 | 1.06-1.81 | 0.02 |
| | | | | | | | | | | | 19.9 | 1.72 | 1.35-2.20 | 1E−05 |
| 026 | 3 | 0.65 | 2 | 0.31 | 2 | 0.18 | 457 | 650 | 1122 | | | | | |
| 028 | 3 | 0.65 | | 0.00 | 1 | 0.09 | 457 | 652 | 1123 | | | | | |
| 029 | | 0.00 | | 0.00 | 1 | 0.09 | 460 | 652 | 1123 | | | | | |
| 030 | | 0.00 | 1 | 0.15 | 1 | 0.09 | 460 | 651 | 1123 | | | | | |
| 035 | 2 | 0.43 | 4 | 0.61 | 13 | 1.16 | 458 | 648 | 1111 | | | | | |
| 036 | 10 | 2.17 | 16 | 2.45 | 16 | 1.42 | 450 | 636 | 1108 | | | | | |
| 038 | | 0.00 | 1 | 0.15 | 5 | 0.44 | 460 | 651 | 1119 | | | | | |
| 041 | | 0.00 | | 0.00 | 1 | 0.09 | 460 | 652 | 1123 | | | | | |
| 046 | 2 | 0.43 | 2 | 0.31 | 2 | 0.18 | 458 | 650 | 1122 | | | | | |

| | | | | | 1124 | | | | | | | | | |
|---|---|---|---|---|---|---|---|---|---|---|---|---|---|---|
| DQA1 | 460 | % | 652 | % | cont | % | c | d | d | | | | | |
| 001 | 214 | 46.52 | 300 | 46.01 | 493 | 43.86 | 246 | 352 | 631 | | | | | |
| 002 | 34 | 7.39 | 44 | 6.75 | 128 | 11.39 | 426 | 608 | 996 | | | | | |
| 003 | 31 | 6.74 | 50 | 7.67 | 87 | 7.74 | 429 | 602 | 1037 | | | | | |
| 004 | 77 | 16.74 | 135 | 20.71 | 241 | 21.44 | 383 | 517 | 883 | x | 4.21 | 0.74 | 0.55-0.99 | 0.04 |
| 00402 | | 0.00 | 2 | 0.31 | 4 | 0.36 | 460 | 650 | 1120 | | | | | |
| 005 | 90 | 19.57 | 133 | 20.40 | 247 | 21.98 | 370 | 519 | 877 | | | | | |
| 006 | 205 | 44.57 | 227 | 34.82 | 357 | 31.76 | 255 | 425 | 767 | x | 10.38 | 1.51 | 1.17-1.94 | 0.002 |
| 007 | | 0.00 | 1 | 0.15 | 1 | 0.09 | 460 | 651 | 1123 | | | | | |
| 008 | | 0.00 | | 0.00 | 1 | 0.09 | 460 | 652 | 1123 | | | | | |
| 009 | 76 | 16.52 | 113 | 17.33 | 170 | 15.12 | 384 | 539 | 954 | | | | | |
| 010 | 2 | 0.43 | 4 | 0.61 | 7 | 0.62 | 458 | 648 | 1117 | | | | | |
| 012011 | 1 | 0.22 | | 0.00 | 1 | 0.09 | 459 | 652 | 1123 | | | | | |
| 012012 | 1 | 0.22 | 4 | 0.61 | 12 | 1.07 | 459 | 648 | 1112 | | | | | |
| 014 | 2 | 0.43 | | 0.00 | 1 | 0.09 | 458 | 652 | 1123 | | | | | |

| DRB1 | 460 | % | 652 | % | 1124 cont | % | c | d | d | | | | | | |
|---|---|---|---|---|---|---|---|---|---|---|---|---|---|---|---|
| 001 | 119 | 25.87 | 196 | 30.06 | 278 | 24.73 | 341 | 456 | 846 | | | | | | |
| 00103 | 1 | 0.22 | 1 | 0.15 | 1 | 0.09 | 459 | 651 | 1123 | | | | | | |
| 002 | 57 | 12.39 | 55 | 8.44 | 92 | 8.19 | 403 | 597 | 1032 | x | 4.23 | 1.54 | 1.02-2.31 | 0.04 | |
| | | | | | | | | | | | 6.29 | 1.59 | 1.1-2.28 | 0.01 | |
| 00203 | | 0.00 | 4 | 0.61 | 4 | 0.36 | 460 | 648 | 1120 | | | | | | |
| 003 | 1 | 0.22 | 1 | 0.15 | 3 | 0.27 | 459 | 651 | 1121 | | | | | | |
| 004 | 1 | 0.22 | 6 | 0.92 | 44 | 3.91 | 459 | 646 | 1080 | | | | | | |
| 005 | 5 | 1.09 | 13 | 1.99 | 14 | 1.25 | 455 | 639 | 1110 | | | | | | |
| 006 | 96 | 20.87 | 146 | 22.39 | 288 | 25.62 | 364 | 506 | 836 | x | ns | | | | |
| | | | | | | | | | | | ns | | | | |
| 008 | 1 | 0.22 | 18 | 2.76 | 32 | 2.85 | 459 | 634 | 1092 | x | 8.93 | 0.08 | 0.00-0.54 | 0.003 | |
| 00802 | 9 | 1.96 | 3 | 0.46 | 7 | 0.62 | 451 | 649 | 1117 | x | 4.34 | 4.32 | 1.07-20.19 | 0.04 | |
| 009 | 61 | 13.26 | 62 | 9.51 | 89 | 7.92 | 399 | 590 | 1035 | x | 3.49 | 1.45 | 0.98-2.15 | 0.06 | ns |
| | | | | | | | | | | | 10.25 | 1.78 | 1.24-2.55 | 0.001 | |
| 010 | 1 | 0.22 | | 0.00 | | 0.00 | 459 | 652 | 1124 | | | | | | |
| 011 | 29 | 6.30 | 26 | 3.99 | 66 | 5.87 | 431 | 626 | 1058 | | | | | | |
| 012 | 47 | 10.22 | 91 | 13.96 | 141 | 12.54 | 413 | 561 | 983 | | | | | | |
| 013 | 41 | 8.91 | 44 | 6.75 | 70 | 6.23 | 419 | 608 | 1054 | | | | | | |
| | | 0.00 | | 0.00 | 1 | 0.09 | 460 | 652 | 1123 | | | | | | |
| 015 | 219 | 47.61 | 255 | 39.11 | 385 | 34.25 | 241 | 397 | 739 | x | 7.62 | 1.41 | 1.10-1.81 | 0.006 | |
| | | | | | | | | | | | 24.12 | 1.74 | 1.39-2.19 | 1E−06 | |
| 01503 | | 0.00 | 1 | 0.15 | 24 | 2.14 | 460 | 651 | 1100 | | | | | | |
| 016 | | 0.00 | 3 | 0.46 | 6 | 0.53 | 460 | 649 | 1118 | | | | | | |
| 017 | 2 | 0.43 | 2 | 0.31 | 3 | 0.27 | 458 | 650 | 1121 | | | | | | |
| 018 | 16 | 3.48 | 34 | 5.21 | 61 | 5.43 | 444 | 618 | 1063 | | | | | | |
| 019 | 1 | 0.22 | 1 | 0.15 | 3 | 0.27 | 459 | 651 | 1121 | | | | | | |
| 020 | 25 | 5.43 | 44 | 6.75 | 77 | 6.85 | 435 | 608 | 1047 | | | | | | |
| 023 | 13 | 2.83 | 11 | 1.69 | 14 | 1.25 | 447 | 641 | 1110 | | | | | | |
| 024 | | 0.00 | | 0.00 | 4 | 0.36 | 460 | 652 | 1120 | | | | | | |
| 025 | 1 | 0.22 | 4 | 0.61 | 12 | 1.07 | 459 | 648 | 1112 | | | | | | |
| 028 | | 0.00 | | 0.00 | 1 | 0.09 | 460 | 652 | 1123 | | | | | | |
| 029 | | 0.00 | 1 | 0.15 | 1 | 0.09 | 460 | 651 | 1123 | | | | | | |
| 03202 | | 0.00 | | 0.00 | 1 | 0.09 | 460 | 652 | 1123 | | | | | | |
| 033 | | 0.00 | 1 | 0.15 | 5 | 0.44 | 460 | 651 | 1119 | | | | | | |
| 040 | 2 | 0.43 | 4 | 0.61 | 7 | 0.62 | 458 | 648 | 1117 | | | | | | |
| 046 | | 0.00 | | 0.00 | 6 | 0.53 | 460 | 652 | 1118 | | | | | | |
| 047 | | 0.00 | 1 | 0.15 | 3 | 0.27 | 460 | 651 | 1121 | | | | | | |
| 048 | | 0.00 | 3 | 0.46 | 4 | 0.36 | 460 | 649 | 1120 | | | | | | |
| 052 | 1 | 0.22 | | 0.00 | 1 | 0.09 | 459 | 652 | 1123 | | | | | | |
| 053 | | 0.00 | | 0.00 | 1 | 0.09 | 460 | 652 | 1123 | | | | | | |
| 054 | | 0.00 | 1 | 0.15 | 1 | 0.09 | 460 | 651 | 1123 | | | | | | |
| 069 | | 0.00 | | 0.00 | 16 | 1.42 | 460 | 652 | 1108 | | | | | | |
| 071 | | 0.00 | 3 | 0.46 | 3 | 0.27 | 460 | 649 | 1121 | | | | | | |
| 073 | 1 | 0.22 | 4 | 0.61 | 5 | 0.44 | 459 | 648 | 1119 | | | | | | |
| 075 | | 0.00 | 2 | 0.31 | 2 | 0.18 | 460 | 650 | 1122 | | | | | | |
| a79 | | 0.00 | | 0.00 | 1 | 0.09 | 460 | 652 | 1123 | | | | | | |
| a79v | | 0.00 | | 0.00 | 1 | 0.09 | 460 | 652 | 1123 | | | | | | |
| Ik3389 | 2 | 0.43 | 2 | 0.31 | 2 | 0.18 | 458 | 650 | 1122 | | | | | | |
| New | 3 | 0.65 | 6 | 0.92 | 11 | 0.98 | 457 | 646 | 1113 | | | | | | |
| Ik3385 | 1 | 0.22 | | 0.00 | | 0.00 | 459 | 652 | 1124 | | | | | | |
| n3315 | 1 | 0.22 | | 0.00 | | 0.00 | 459 | 652 | 1124 | | | | | | |

| | | | 460 diab | | 652 | | 1124 | | | | | | | | | |
|---|---|---|---|---|---|---|---|---|---|---|---|---|---|---|---|---|
| DRB1 | DQA1 | DQB1 | no Fe | % | matcon | % | cont | % | c | d | d | | | | | |
| | 001 | 002 | 160 | 34.63 | 244 | 37.42 | 372 | 33.10 | 300 | 408 | 752 | | | | | |
| | 001 | 008 | 64 | 13.85 | 76 | 11.66 | 155 | 13.79 | 396 | 576 | 969 | xx | ns | | | |
| 001 | 001 | 036 | 10 | 2.16 | 16 | 2.45 | 16 | 1.42 | 450 | 636 | 1108 | | | | | |
| | 002 | 013 | 33 | 7.14 | 37 | 5.67 | 80 | 7.12 | 427 | 615 | 1044 | | | | | |
| | 002 | 015 | 2 | 0.43 | 6 | 0.92 | 46 | 4.09 | 458 | 646 | 1078 | xx | 13.64 | 0.1 | 0.02-0.43 | 0.0002 |
| | 003 | 004 | 14 | 3.03 | 29 | 4.45 | 56 | 4.98 | 446 | 623 | 1068 | | | | | |
| | 003 | 005 | 18 | 3.90 | 24 | 3.68 | 30 | 2.67 | 442 | 628 | 1094 | | | | | |
| | 004 | 013 | 40 | 8.66 | 70 | 10.74 | 143 | 12.72 | 420 | 582 | 981 | xx | 4.79 | 0.65 | 0.44-0.96 | 0.03 |
| 012 | 004 | 013017 | 38 | 8.23 | 75 | 11.50 | 109 | 9.70 | 422 | 577 | 1015 | | | | | |
| 006 | 005 | 007 | 82 | 17.75 | 121 | 18.56 | 232 | 20.64 | 378 | 531 | 892 | | | | | |
| 006 | 005 | 02001 | 8 | 1.73 | 22 | 3.37 | 24 | 2.14 | 452 | 630 | 1100 | | | | | |
| 006 | 005 | 028 | 3 | 0.65 | | | 1 | 0.09 | 457 | 652 | 1123 | | | | | |
| 015 | 006 | 003 | 7 | 1.52 | 15 | 2.30 | 27 | 2.40 | 453 | 637 | 1097 | | | | | |
| 01503 | 006 | 003v | | | | | 23 | 2.05 | 460 | 652 | 1101 | | | | | |
| 015 | 006 | 011 | 5 | 1.08 | | | | 0.00 | 455 | 652 | 1124 | | | | | |
| 015 | 006 | 019022 | 12 | 2.60 | 8 | 1.23 | 8 | 0.71 | 448 | 644 | 1116 | | | | | |
| 015 | 006 | 02002 | 25 | 5.41 | 26 | 3.99 | 36 | 3.20 | 435 | 626 | 1088 | | | | | |
| 015 | 006 | 022 | 13 | 2.81 | 12 | 1.84 | 14 | 1.25 | 447 | 640 | 1110 | | | | | |
| | 006 | 023 | 158 | 34.20 | 177 | 27.15 | 260 | 23.13 | 302 | 475 | 864 | x | 6.31 | 1.4 | 1.08-1.83 | 0.01 |
| | | | | | | | | | | | | | 20.57 | 1.74 | 1.36-2.22 | 0.000006 |
| | 009 | 001 | 77 | 16.67 | 112 | 17.18 | 171 | 15.21 | 383 | 540 | 953 | | | | | |
| 040 | 010 | 019 | 2 | 0.43 | 4 | 0.61 | 7 | 0.62 | 458 | 648 | 1117 | | | | | |
| 025 | 012012 | 035 | 1 | 0.22 | 4 | 0.61 | 12 | 1.07 | 459 | 648 | 1112 | | | | | |
| Other | | | 13 | 2.81 | 16 | 2.45 | 34 | 3.02 | 447 | 636 | 1090 | | | | | |
| rare | | | | | | | | | | | | | | | | |
| haplos | | | | | | | | | | | | | | | | |

Allelic names and sequences for class II alleles are shown below:

DLA dqa1.L12, exon 2 (nucleotides 15-260)
>DQA1*00101
GAC CAT GTT GCC AAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT AGA AGT TTT GAC CCA CAG GGT GCA CTG AGA AAC
TTG GCT ATA ATA AAA CAA AAC TTG AAC ATC ATG ACT
AAA AGG TCC AAC CAA ACT GCT GCT ACC AAT
>DQA1*00201
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCT ATA ACA AAA CAA AAC TTG AAC ATC ATG ACT
AAA AGG TCC AAC AAA ACT GCT GCT ACC AAT
>DQA1*00301
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCC AGA GCA AAA CAA AAC TTG AAC ATC CTG ACT
AAA AGT TCC AAC CAA ACT GCT GCT ACC AAT
>DQA1*00401
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCT ATA ATA AAA CAA AAC TTG AAC ATC CTG ACT
AAA AGG TCC AAC CAA ACT GCT GCT ACC AAT
>DQA1*005011
GAC CAT GTT GCC TAC TAC GGC ATA AAT GTC TAC CAG
TCT TAC GGT CCC TCT GGC CAG TTC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCT ATA ACA AAA CAA AAC TTG AAC ATC ATG ACT
AAA AGG TCC AAC AAA ACT GCT GCT ACC AAT
>DQA1*005012
GAC CAT GTT GCC TAC TAC GGC ATA AAT GTC TAC CAG
TCT TAC GGT CCC TCT GGC CAG TTC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCG CTG AGA AAC
TTG GCT ATA ACA AAA CAA AAC TTG AAC ATC ATG ACT
AAA AGG TCC AAC AAA ACT GCT GCT ACC AAT
>DQA1*00601
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT AGA AGT TTT GAC CCA CAG GGT GCA CTG AGA AAC
TTG GCT ATA ATA AAA CAA AAC TTG AAC ATC CTG ACT
AAA AGG TCC AAC CAA ACT GCT GCT ACC AAT
>DQA1*00701
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCT ATA ACA AAA CAA AAC TTG AAC ATC ATG ACT
AAA AGG TCC AAC CAA ACT GCT GCT ACC AAT
>DQA1*00801
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCC AGA GCA AAA CAA AAC TTG AAC ATC CTG ACT
AAA AGG TCC AAC CAA ACT GCT GCT ACC AAT
>DQA1*00901
GAC CAT GTT GCC TAC TAC GGC ATA AAT GTC TAC CAG
TCT TAC GGT CCC TCT GGC CAG TTC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT AGA AGT TTT GAC CCA CAG GGT GCA CTG AGA AAC
TTG GCT ATA ATA AAA CAA AAC TTG AAC ATC ATG ACT
AAA AGG TCC AAC CAA ACT GCT GCT ACC AAT
>DQA1*01001
GACCATGTTGCCTACTACGGCATAAATGTCTACCAGTCTTACGGTCCCTC
TGGCCAGTACACCCATGAATTTGATGGCGATGAGGAGTTCTACGTGGACC
TGGAGAAGAAGGAAACTGTCTGGCGGCTGCCTGTGTTTAGCACATTTAGA
AGTTTTGACCCACAGGGTGCACTGAGAAACTTGGCTATAGCAAAACAAAA
CTTGAACATCCTGACTAAAAGTTCCAACCAAACTGCTGCTACCAAT
>DQA1*01101
GACCATGTTGCCTACTACGGCATAAATGTCTACCAGTCTTACGGTCCCTC
TGGCCAGTACACCCATGAATTTGATGGCGATGAGGAGTTCTACGTGGACC
TGGAGAAGAAGGAAACTGTCTGGCGGCTGCCTGTGTTTAGCACATTTACA
AGTTTTGACCCACAGGGTGCACTGAGAAACTTGGCTATAATAAAACAAAA
CTTGAACATCATGACTAAAAGGTCCAACAAAACTGCTGCTACCAAT
>DQA1*012011
GACCATGTTGCCTACTACGGCATAAATGTCTACCAGTCTTACGGTCCCTC
TGGCCAGTACACCCATGAATTTGATGGCGATGAGGAGTTCTACGTGGACC
TGGAGAAGAAGGAAACTGTCTGGCGGCTGCCTGTGTTTAGCACATTTGCA
AGTTTTGACCCACAGGGTGCACTGAGAAACTTGGCTATAGCAAAACAAAA
CTTGAACATCATGACTAAAAGGTCCAACCAAACTGCTGCTACCAAT
>dqa1*012012
GACCATGTTGCCTACTACGGCATAAATGTCTACCAGTCTTACGGTCCCTC
TGGCCAGTACACCCATGAATTTGATGGCGATGAGGAaTTCTACGTGGACC
TGGAGAAGAAGGAAACTGTCTGGCGGCTGCCTGTGTTTAGCACATTTGCA
AGTTTTGACCCACAGGGTGCACTGAGAAACTTGGCTATAGCAAAACAAAA
CTTGAACATCATGACTAAAAGGTCCAACCAAACTGCTGCTACCAAT
>DQA1*01301
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT AGA AGT TTT GAC CCA CAG GGT GCA CTG AGA AAC
TTG GCT ATA ACA AAA CAA AAC TTG AAC ATC ATG ACT
AAA AGG TCC AAC AAA ACT GCT GCT ACC AAT
>DQA1*014011
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT AGA AGT TTT GAC CCA CAG GGT GCA CTG AGA AAC
TTG GCT ATA ATA AAA CAA AAC TTG AAC ATC ATG ACT
AAA AGG TCC AAC CAA ACT GCT GCT ACC AAT
>DQA1*014012
GAC CAT GTT GCC TAC TAC GGC ATA AAT GTC TAC CAG
TCT TAC GGT CCC TCT GGC CAG TAC ACA CAT GAA TTT
GAT GGC GAT GAG GAG TTC TAC GTG GAC CTG GAG AAG
AAG GAA ACT GTC TGG CGG CTG CCT GTG TTT AGC ACA
TTT AGA AGT TTT GAC CCA CAG GGT GCA CTG AGA AAC
TTG GCT ATA ATA AAA CAA AAC TTG AAC ATC ATG ACT
AAA AGG TCC AAC CAA ACT GCT GCT ACC AAT
>DQA1*01501
GACCATGTTGCCAACTACGGCATAAATGTCTACCAGTCTTACGGTCCCTC
TGGCCAGTTCACCCATGAATTTGATGGCGATGAGGAGTTCTACGTGGACC
TGGAGAAGAAGGAAACTGTCTGGCGGCTGCCTGTGTTTAGCACATTTAGA
AGTTTTGACCCACAGGGTGCACTGAGAAACTTGGCTATAATAAAACAAAA
CTTGAACATCATGACTAAAAGGTCCAACCAAACTGCTGCTACCAAT
>07v1
GAC CAT GTT GCC TAC TAC GGC ATA AAT GTC TAC CAG
TCT TAC GGT CCC TCT GGC CAG TTC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCT ATA ACA AAA CAA AAC TTG AAC ATC ATG ACT
AAA AGG TCC AAC CAA ACT GCT GCT ACC AAT
>dqa1*00402
GACCATGTTGCCTACTACGGCATAAATGTCTACCAGTCTTACGGTCCCTC
TGGCCAGTACACCCATGAATTTGATGGCGATGAGttGTTCTACGTGGACC
TGGAGAAGAAGGAAACTGTCTGGCGGCTGCCTGTGTTTAGCACATTTACA
AGTTTTGACCCACAGGGTGCACTGAGAAACTTGGCTATAATAAAACAAAA
CTTGAACATCCTGACTAAAAGGTCCAACCAAACTGCTGCTACCAAT
>dqa383-11
GACCATGTTGCCAACTACGGCATAAATGTCTACCAGTCTTACGGTCCCTC
TGGCCAGTACACCCATGAATTTGATGGCGATGAGGAGTTCTACGTGGACC
TGGAGAAGAAGGAAACTGTCTGGCGGCTGCCTGTGTTTAGCACATTTACA
AGTTTTGACCCACAGGGTGCACTGAGAAACTTGGCCATAACAAAACAAAA
CTTGAACATCATGACTAAAAGGTCCAACAAAACTGCTGCTACCAAT
>DQA1*01601
GAC CAT GTT GCC TAC TAC GGC ATA AAT GTC TAC CAG
TCT TAC GGT CCC TCT GGC CAG TAC ACA CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCT ATA ATA AAA CAA AAC TTG AAC ATC ATG ACT
AAA AGG TCC AAC CAA ACT GCT GCT ACC AAT
>DQA1*01602
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAT ACT GTC TGG CGG CTG CCT GTG TTT AGC ACA
TTT ACA AGT TTT GAC CCA CAG GGT GCA CTG AGA AAC
TTG GCT ATA ATA AAA CAA AAC TTG AAC ATC ATG ACT
AAA AGG TCC AAC CAA ACT GCT GCT ACC AAT
>DQA/M/LO51
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCT ATA GCA AAA CAA AAC TTG AAC ATC CTG ACT
AAA AGT TCC AAC CAA ACT GCT GCT ACC AAT
>DQA/W53/B
GAC CAT GTT GCC aAC TAC GGC ATA AAT GTC TAC CAG
TCT TAC GGT CCC TCT GGC CAG TaC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCT ATA AtA AAA CAA AAC TTG AAC ATC ATG ACT
AAA AGG TCC AAC cAA ACT GCT GCT ACC AAT
>DQA1*01701
GAC CAT GTT GCC TAC TAC GGC ATA AAT GTC TAC CAG
TCT TAC GGT CCC TCT GGC CAG TAC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT GCA AGT TTT GAC CCA CAG GGT GCA CTG AGA AAC
TTG GCT AGA GCA AAA CAA AAC TTG AAC ATC CTG ACT
AAA AGT TCC AAC CAA ACT GCT GCT ACC AAT
>DQA/COY954A
GAC CAT GTT GCC TAC TAC GGC ATA AAT GTC TAC CAG
TCT TAC GGT CCC TCT GGC CAG TTC ACC CAT GAA TTT
GAT GGC GAT GAG GAG TTC TAC GTG GAC CTG GAG AAG
AAG GAA ACT GTC TGG CGG CTG CCT GTG TTT AGC ACA
TTT ACA AGT TTT GAC CCA CAG GGT GCA CTG AGA AAC
TTG GCT ATA ATA AAA CAA AAC TTG AAC ATC ATG ACT
AAA AGG TCC AAC CAA ACT GCT GCT ACC AAT
>hcdqa-1DM
GACCATGTTGCCTACTACGGCATAAATGTCTACCAGTCTTACGGTCCCTC
TGGCCAGTACACCCATGAATTTGATGGCGATGAGGAGTTCTACGTGGACC
TGGAGAAGAAGGAAACTGTCTGGCGGCTGCCTGTGTTTAGCACATTTACA
AGTTTTGACCCACAGGGTGCACTGAGAAACTTGGCTATAgCAAAACAAAA
CTTGAACATCATGACTAAAAGGTCCAACAAAACTGCTGCTACCAAT
>awddqa01
GACCATGTTGCCAACTACGGCATAAATGTCTACCAGTCTTACGGTCCCTC
TGGCCAGTTCACCCATGAATTTGATGGCGATGAGGAGTTCTATGTGGACC
TGGAGAAGAAGGAAACTGTCTGGCGGCTGCCTGTGTTTAGCACATTTAGA
AGTTTTGACCCACAGGGTGCACTGAGAAACTTGGCTATAATAAAACAAAA
CTTGAACATCCTGACTAAAAGGTCCAaCCAAAcTGCtGCTaCCAaT
>dqa-1k-ew73
GACCATGTTGCCTACTACGGCATAAATGTCTACCAGTCTTACGGTCCCTC
TGGCCAGTACACCCATGAATTTGATGGCGATGAGttGTTCTACGTGGACC
TGGAGAAGAAGGAAACTGTCTGGCGGCTGCCTGTGTTTAGCACATTTACA
AGTTTTGACCCACAGGGTGCACTGAGAAACTTGGCTATAgcAAAACAAAA
CTTGAACATCCTGACTAAAAGGTCCAACCAAACTGCTGCTACCAAT
DLA-DQB1 (base 1 = base 16 of exon 2)
>DQB1*00101
GATTTCGTGTACCAGTTTAAGGGCGAGTGCTATTTCACCAACGGGACGGA
GCGGGTGCGGCTTCTGACGAGAGACATCTATAACCGGGAGGAGCACGTGC
GCTTCGACAGCGACGTGGGGGAGTACCGGGCGGTCACGGAGCTCGGGCGG
CCGGACGCTGAGTACTGGAACGGGCAGAAGGAGCTCTTGGAGCGGAGGCG
GGCCGAGGTGGACACGGTGTGCAGACACAACTACGGGAGGGAAGAGCTCA
CCACGTTGCAGCGGCGA
>DQB1*00202
GATTTCGTGTTCCAGTATAAGGCCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTTCCGGGCGGTCACGGAGCTCGGGCGG
CCCGACGCTGAGTACTGGAACCGACAGAAGGACGAGATGGACCGGGTACG
GGCCGAGCTGGACACGGTGTGCAGACACAACTACGGGAGGGAAGAGCTCA
CCACGTTGCAGCGGCGA
>DQB1*00301
GATTTCGTGTACCAGTTTAAGGCCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTACCGGGCGGTCACGGAGCTCGGGCGG
CCCTCGGCTGAGTACTGGAACCCGCAGAAGGACGAGATGGACCGGGTACG
GGCCGAGCTGGACACGGTGTGCAGACACAACTACGGGTTGGAAGAGCTCA
CCACGTTGCAGCGGCGA
>DQB1*00401
GATTTCGTGTTCCAGTTTAAGGGCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTACCGGGCGGTCACGGAGCTCGGGCGG
CCCTGGGCTGAGTACTGGAACCCGCAGAAGGACGAGATGGACCGGGTACG
GGCCGAGCTGGACACGGTGTGCAGACACAACTACGGGTTGGAAGAGCTCT
ACACGTTGCAGCGGCGA
>DQB1*00501
GATTTCGTGTTCCAGTATAAGGCCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTACCGGGCGGTCACGGAGCTCGGGCGG
CCCTGGGCTGAGTACTGGAACCCGCAGAAGGACGAGATGGACCGGGTACG
GGCCGAGCTGGACACGGTGTGCAGACACAACTACGGGTTGGAAGAGCTCA
CCACGTTGCAGCGGCGA
>DQB1*00502
GATTTCGTGTTCCAGTATAAGGCCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTTCCGGGCGGTCACGGAGCTCGGGCGG
CCCTGGGCTGAGTACTGGAACCCGCAGAAGGACGAGATGGACCGGGTACG
GGCCGAGCTGGACACGGTGTGCAGACACAACTACGGGTTGGAAGAGCTCA
CCACGTTGCAGCGGCGA
>DQB1*00701
GATTTCGTGTACCAGTTTAAGTTCGAGTGCTATTTCACCAACGGGACGGA
GCGGGTGCGGCTTCTGGCGAGAGACATCTATAACCGGGAGGAGTTCGTGC
GCTTCGACAGCGACGTGGGGGAGTACCGGGCGGTCACGGAGCTCGGGCGG
CCGGACGCTGAGTACTGGAACGGGCAGAAGGAGCTCTTGGAGCAGAGGCG
GGCCGAGCTGGACACGGTGTGCAGACACAACTACGGGTTGGAAGAGCTCT
ACACGTTGCAGCGGCGA
>DQB1*008011
GATTTCGTGTACCAGTTTAAGGGCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCGACAGAAGGACGAGATGGACCGGGTACG
GGCCGAGCTGGACACGGTGTGCAGACACAACTACGGGAGGGAAGAGCTCA
CCACGTTGCAGCGGCGA
>DQB1*008012
GATTTCGTGTACCAGTTTAAGGGCGAGTGCTATTTCACCAACGGGACGGA
GCGGGTGCGGCTTCTGACTAAATACATCTATAACCGGGAGGAGTTCGTGC
GCTTTGACAGCGACGTGGGGGAGTACCGGGCGGTCACGGAGCTCGGGCGG
CCCGACGCTGAGTACTGGAACCGACAGAAGGACGAGATGGACCGGGTACG
GGCCGAGCTGGACACGGTGTGCAGACACAACTACGGGAGGGAAGAGCTCA
CCACGTTGCAGCGGCGA
>DQB1*00802
GATTTCGTGTACCAGTTTAAGGGCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTTCCGGGCGGTCACGGAGCTCGGGCGG
CCCGACGCTGAGTACTGGAACCGACAGAAGGACGAGATGGACCGGGTACG
GGCCGAGCTGGACACGGTGTGCAGACACAACTACGGGAGGGAAGAGCTCA
CCACGTTGCAGCGGCGA
>DQB1*01101
GATTTCGTGTACCAGTTTAAGGCCGAGTGCTATTTCACCAACGGGACGGA
GCGGGTGCGGCTTCTGACTAAATACATCTATAACCGGGAGGAGCACGTGC
GCTTCGACAGCGACGTGGGGGAGTACCGGGCGGTCACGGAGCTCGGGCGG
CCCTCGGCTGAGTACTGGAACCCGCAGAAGGACGAGATGGACCGGGTACG
GGCCGAGCTGGACACGGTGTGCAGACACAACTACGGGTTGGAAGAGCTCA
CCACGTTGCAGCGGCGA
>DQB1*01201
GATTTCGTGTTCCAGTTTAAGTTCGAGTGCTATTTCACCAACGGGACGGA
GCGGGTGCGGCTTCTGGCGAGAGACATCTATAACCGGGAGGAGTTCGTGC
GCTTCGACAGCGACGTGGGGGAGTACCGGGCGGTCACGGAGCTCGGGCGG
CCGGACGCTGAGTACTGGAACGGGCAGAAGGAGCTCTTGGAGCAGAGGCG
GGCCGAGCTGGACACGGTGTGCAGACACAACTACGGGTTGGAAGAGCTCT
ACACGTTGCAGCGGCGA
>DQB1*01301
GATTTCGTGTACCAGTTTAAGTTCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCCGCAGAAGGACGAGATGGACCGGGTACG
GGCCGAGCTGGACACGGTGTGCAGACACAACTACGGGAGGGAAGAGCTCA
CCACGTTGCAGCGGCGA
>DQB1*01302
GATTTCGTGTACCAGTTTAAGTTCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCCGCAGAAGGACGAGATGGACCGGGTACG
GGCCGAGCTGGACACGGTGTGCAGACACAACTACGGGGTGGAAGAGCTCA
CCACGTTGCAGCGGCGA
>DQB1*01303
GATTTCGTGTACCAGTTTAAGTTCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCCGCAGAAGGACGAGATGGACCGGGTACG
GGCCGAGCTGGACACGGTGTGCAGACACAACTACGGGGTGGAAGAGCTCT
ACACGTTGCAGCGGCGA
>DQB1*01304
GATTTCGTGTACCAGTTTAAGTTCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTTCCGGGCGGTCACGGAGCTCGGGCGG
CCCGACGCTGAGTACTGGAACCCGCAGAAGGACGAGATGGACCGGGTACG
GGCCGAGCTGGACACGGTGTGCAGACACAACTACGGGGTGGAAGAGCTCT
ACACGTTGCAGCGGCGA
>DQB1*01401
GATTTCGTGTACCAGTTTAAGGGCGAGTGCTATTTCACCAACGGGACGGA
GCGGGTGCGGCTTCTGGCGAGAGACATCTATAACCGGGAGGAGCACGTGC
GCTTCGACAGCGACGTGGGGGAGTTCCGGGCGGTCACGGAGCTCGGGCGG
CCCTGGGCTGAGTACTGGAACGGGCAGAAGGAGATCTTGGAGCGGAAGCG
GGCCGAGCTGGACACGGTGTGCAGACACAACTACGGGGTGGAAGAGCTCT
ACACGTTGCAGCGGCGA
>DQB1*01501
GATTTCGTGTACCAGTGTAAGGCCGAGTGCTATTTCACCAACGGGACGGA
GCGGGTGCGGTTTCTGGCTAAATACATCTATAACCGGGAGGAGTTCGTGC
GCTTCGACAGCGACGTGGGGGAGTTCCGGGCGGTCACGGAGCTCGGGCGG
CCCTCGGCTGAGTACTGGAACGGGCAGAAGGAGATCTTGGAGCAGGAGCG
GGCAACGGTGGACACGGTGTGCAGACACAACTACGGGGTGGAAGAGCTCT
ACACGTTGCAGCGGCGA
>DQB1*01601
GATTTCGTGTACCAGTTTAAGGGCGAGTGCTATTTCACCAACGGGACGGA
GCGGGTGCGGCTTCTGACGAGAAGCATCTATAACCGGGAGGAGTTCGTGC
GCTTCGACAGCGACGTGGGGGAGTTCCGGGCGGTCACGGAGCTCGGGCGG
CCCGTCGCTGAGTACTGGAACGGGCAGAAGGAGATCTTGGAGCGGAAGCG
GGCCGAGGTGGACAGGGTGTGCAGACACAACTACGGGAGGGAAGAGCTCA
CCACGTTGCAGCGGCGA
>DQB1*01701
GATTTCGTGTTCCAGTGTAAGGCCGAGTGCTATTTCACCAACGGGACGGA
GCGGGTGCGGTTTCTGGCTAAATACATCTATAACCGGGAGGAGTTCGTGC
GCTTCGACAGCGACGTGGGGGAGTACCGGGCGGTCACGGAGCTCGGGCGG
CCCGACGCTGAGTCCTGGAACGGGCAGAAGGAGTTCTTGGAGCAGGAGCG
GGCAACGGTGGACACGGTGTGCAGACACAACTACGGGGTGGAAGAGCTCT
ACACGTTGCAGCGGCGA
>DQB1*01801
GATTTCGTGTACCAGTTTAAGTTCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCCGCAGAAGGACGAGATGGACCGGGTGCG
GGCCGAGGTGGACACGGTGTGCAGACACAACTACGGGGTGGAAGAGCTCT
CCACGTTGCAGCGGCGA
>DQB1*01901
GATTTCGTGTTCCAGTGTAAGGCCGAGTGCTATTTCACCAACGGGACGGA
GCGGGTGCGGTTTCTGGCTAAATACATCTATAACCGGGAGGAGTTCGTGC
GCTTCGACAGCGACGTGGGGGAGTTCCGGGCGGTCACGGAGCTCGGGCGG
CCCTCGGCTGAGTACTGGAACGGGCAGAAGGAGCTCTTGGAGCAAGAGCG
GGCAACGGTGGACACGGTGTGCAGACACAACTACGGGAGGGAAGAGCTCA
CCACGTTGCAGCGGCGA
>DQB1*02001
GATTTCGTGTACCAGTTTAAGGCCGAGTGCTATTTCACCAACGGGACGGA
GCGGGTGCGGCTTCTGACGAGAAGCATCTATAACCGGGAGGAGTTCGTGC
GCTTCGACAGCGACGTGGGGGAGTTCCGGGCGGTCACGGAGCTCGGGCGG
CCCGTCGCTGAGTACTGGAACGGGCAGAAGGAGATCTTGGAGCGGAAGCG
GGCCGCGGTGGACACGGTGTGCAGACACAACTACGGGAGGGAAGAGCTCA
CCACGTTGCAGCGGCGA
>DQB1*02002
GATTTCGTGTACCAGTTTAAGGCCGAGTGCTATTTCACCAACGGGACGGA
GCGGGTGCGGCTTCTGACGAGAAGCATCTATAACCGGGAGGAGTTCGTGC
GCTTCGACAGCGACGTGGGGGAGTTCCGGGCGGTCACGGAGCTCGGGCGG
CCCGTCGCTGAGTACTGGAACGGGCAGAAGGAGATCTTGGAGCGGAAGCG
GGCCGCGGTGGACAGGGTGTGCAGACACAACTACGGGAGGGAAGAGCTCA
CCACGTTGCAGCGGCGA
>DQB1*02101
GATTTCGTGTACCAGTTTAAGGCCGAGTGCTATTTCACCAACGGGACGGA
GCGGGTGCGGCTTCTGACGAGAAGCATCTATAACCGGGAGGAGTTCGTGC
GCTTCGACAGCGACGTGGGGGAGTTCCGGGCGGTCACGGAGCTCGGGCGG
CCCGTCGCTGAGTACTGGAACGGGCAGAAGGAGATCTTGGAGCGGAAGCG
GGCCGAGGTGGACACGGTGTGCAGACACAACTACGGGAGGGAAGAGCTCA
CCACGTTGCAGCGGCGA
>DQB1*02201
GATTTCGTGTACCAGTTTAAGGGCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCTCGGCTGAGTACTGGAACCCGCAGAAGGACGAGATGGACCGGGTACG
GGCCGAGCTGGACACGGTGTGCAGACACAACTACGGGTTGGAAGAGCTCA
CCACGTTGCAGCGGCGA
>DQB1*02301
GATTTCGTGTACCAGTTTAAGGGCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTACCGGGCGGTCACGGAGCTCGGGCGG
CCCTCGGCTGAGTACTGGAACCCGCAGAAGGACGAGATGGACCGGGTACG
GGCCGAGCTGGACACGGTGTGcAGACACAACTACGGGTTGGAAGAGCTCA
CCACGTTGCAGCGGCGA
>DQB1*02302
GATTTCGTGTACCAGTTTAAGGGCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTACCGGGCGGTCACGGAGCTCGGGCGG
CCCTCGGCTGAGTACTGGAACCCGCAGAAGGACGAGATGGACCGGGTACG
GGCCGAGCTGGACACGGTGTGcAGACACAACTACGGGGTGGAAGAGCTCT
ACACGTTGCAGCGGCGA
>DQB1*02401
GATTTCGTGTACCAGTGTAAGGCCGAGTGCTATTTCACCAACGGGACGGA
GCGGGTGCGGTTTCTGGCTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACGGGCAGAAGGAGCTCTTGGAGCAGGAGCG
GGCAACGCTGGACACGGTGTGCAGACACAACTACGGGGTGGAAGAGCTCT
ACACGTTGCAGCGGCGA
>jmadqb-ccah005
GATTTCGTGTTCCAGTGTAAGGCCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCCGCAGAAGGACGAGATGGACCGGGTACG
GGCCGAGCTGGACACGGTGTGCAGACACAACTACGGGTTGGAAGAGCTCA
CCACGTTGCAGCGGCGA
>DQB1*02601
GATTTCGTGTACCAGTTTAAGGGCGAGTGCTATTTCACCAACGGGACGGA
GCGGGTGCGGCTTCTGACGAGAAGCATCTATAACCGGGAGGAGTTCGTGC
GCTTCGACAGCGACGTGGGGGAGTTCCGGGCGGTCACGGAGCTCGGGCGG
CCCGTCGCTGAGTACTGGAACGGGCAGAAGGAGATCTTGGAGCGGAAGCG
GGCCGCGGTGGACAGGGTGTGCAGACACAACTACGGGAGGGAAGAGCTCA
CCACGTTGCAGCGGCGA
>DQB1*02701
GATTTCGTGTACCAGTTTAAGTTCGAGTGCTATTTCACCAACGGGACGGA
GCGGGTGCGGCTTCTGGCGAGAGACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCCGCAGAAGGACGAGATGGACCGGGTACG
GGCCGAGCTGGACACGGTGTGCAGACACAACTACGGGGTGGAAGAGCTCT
ACACGTTGCAGCGGCGA
>DQB1*02801
GATTTCGTGTACCAGTTTAAGTTCGAGTGCTATTTCACCAACGGGACGGA
GCGGGTGCGGCTTCTGGCGAGAGACATCTATAACCGGGAGGAGTTCGTGC
GCTTCGACAGCGACGTGGGGGAGTACCGGGCGGTCACGGAGCTCGGGCGG
CCGGACGCTGAGTACTGGAACCGGCAGAAGGAGCTCTTGGAGCAGAGGCG
GGCCGCGGTGGACACGGTGTGCAGACACAACTACGGGTTGGAAGAGCTCT
ACACGTTGCAGCGGCGA
>DQB1*02901
GATTTCGTGTACCAGTGTAAGGCCGAGTGCTATTTCACCAACGGGACGGA
GCGGGTGCGGTTTCTGGCTAAATACATCTATAACCGGGAGGAGTTCGTGC
GCTTCGACAGCGACGTGGGGGAGTTCCGGGCGGTCACGGAGCTCGGGCGG
CCCGACGCTGAGTACTGGAACGGGCAGAAGGAGCTCTTGGAGCAGGAGCG
GGCAACGGTGGACACGGTGTGCAGACACAACTACGGGGTGGAAGAGCTCT
ACACGTTGCAGCGGCGA
>DQB1*03001
GATTTCGTGTTCCAGTTTAAGGGCGAGTGCTATTTCACCAACGGGACGGA
GCGGGTGCGGCTTCTGACGAGAGACATCTATAACCGGGAGGAGCACGTGC
GCTTCGACAGCGACGTGGGGGAGTACCGGGCGGTCACGGAGCTCGGGCGG
CCGGACGCTGAGTACTGGAACGGGCAGAAGGAGCTCTTGGAGCGGAGGCG
GGCCGAGGTGGACACGGTGTGCAGACACAACTACGGGAGGGAAGAGCTCA
CCACGTTGCAGCGGCGA
>DQB1*03101
GATTTCGTGTTCCAGTTTAAGGGCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTACCGGGCGGTCACGGAGCTCGGGCGG
CCCTCGGCTGAGTACTGGAACCCGCAGAAGGACGAGATGGACCGGGTACG
GGCCGAGCTGGACACGGTGTGCAGACACAACTACGGGTTGGAAGAGCTCA
CCACGTTGCAGCGGCGA
>DQB1*03201
GATTTCGTGTTCCAGTTTAAGTTCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCGACAGAAGGACGAGATGGACCGGGTACG
GGCCGAGCTGGACACGGTGTGCAGACACAACTACGGGAGGGAAGAGCTCA
CCACGTTGCAGCGGCGA
>DQB1*03301
GATTTCGTGTACCAGTTTAAGGCCGAGTGCTATTTCACCAACGGGACGGA
GCGGGTGCGGCTTCTGACGAGAAGCATCTATAACCGGGAGGAGTTGGTGC
GCTTCGACAGCGACGTGGGGGAGTTCCGGGCGGTCACGGAGCTCGGGCGG
CCCGTCGCTGAGTACTGGAACGGGCAGAAGGAGCTCTTGGAGCGGAAGCG
GGCCGCGGTGGACAGGGTGTGCAGACACAACTACGGGAGGGAAGAGCTCA
CCACGTTGCAGCGGCGA
>DQB1*03401
GATTTCGTGTTCCAGTTTAAGGCCGAGTGCTATTTCACCAACGGGACGGA
GCGGGTGCGGCTTCTGACGAGAGACATCTATAACCGGGAGGAGCACGTGC
GCTTCGACAGCGACGTGGGGGAGTACCGGGCGGTCACGGAGCTCGGGCGG
CCCGACGCTGAGTACTGGAACGGGCAGAAGGAGCTCTTGGAGCGGAGGCG
GGCCGAGGTGGACACGGTGTGCAGACACAACTACGGGAGGGAAGAGCTCA
CCACGTTGCAGCGGCGA
>DQB1*03501
GATTTCGTGTACCAGTTTAAGTTCGAGTGCTATTTCACCAACGGGACGGA
GCGGGTGCGGCTTCTGGCGAGAGACATCTATAACCGGGAGGAGCACGTGC
GCTTCGACAGCGACGTGGGGGAGTACCGGGCGGTCACGGAGCTCGGGCGG
CCCGACGCTGAGTACTGGAACGGGCAGAAGGAGCTCTTGGAGCAGAGGCG
GGCCGAGCTGGACACGGTGTGCAGACACAACTACAAGGTTGGAAGAGCTC
TACACGTTGCAGCGGcGA
>DQB1*03601
GATTTCGTGTTCCAGTATAAGGCCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTTCCGGGCGGTCACGGAGCTCGGGCGG
CCCGACGCTGAGTACTGGAACCCGCAGAAGGACGAGATGGACCGGGTACG
GGCCGAGCTGGACACGGTGTGCAGACACAACTACGGGAGGGAAGAGCTCA
CCACGTTGCAGCGGCGA
>dqb1*03701
GATTTCGTGTTCCAGTATAAGGCCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTTCCGGGCGGTCACGGAGCTCGGGCGG
CCCGACGCTGAGTACTGGAACGGGCAGAAGGACGAGATGGACCGGGTACG
GGCCGAGCTGGACACGGTGTGCAGACACAACTACGGGAGGGAAGAGCTCA
CCACGTTGCAGCGGCGA
>1kdqbE18
GATTTCGTGTACCAGTTTAAGGGCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTTCCGGGCGGTCACGGAGCTCGGGCGG
CCGGACGCTGAGTACTGGAACGGGCAGAAGGAGCTCTTGGAGCGGAGGCG
GGCCGAGGTGGACACGGTGTGCAGACACAACTACGGGAGGGAAGAGCTCA
CCACGTTGCAGCGGCGA
>DQB1*03901
GATTTCGTGTTCCAGTTTAAGTTCGAGTGCTATTTCACCAACGGGACGGA
GCGGGTGCGGCTTCTGGCTAAATACATCTATAACCGGGAGGAGTTCGTGC
GCTTCGACAGCGACGTGGGGGAGTACCGGGCGGTCACGGAGCTCGGGCGG
CCCTGGGCTGAGTACTGGAACCCGCAGAAGGACGAGATGGACCGGGTACG
GGCCGAGCTGGACACGGTGTGCAGACACAACTACGGGGTGGAAGAGCTCT
ACACGTTGCAGCGGCGA
>dqbC3007new
GATTTCGTGTACCAGTTTAAGGGCGAGTGCTATTTCACCAACGGGACGGA
GCGGGTGCGGCTTCTGACTAAATACATCTATAACCGGGAGGAGTTGGTGC
GCTTCGACAGCGACGTGGGGGAGTACCGGGCGGTCACGGAGCTCGGGCGG
CCCGACGCTGAGTACTGGAACCGACAGAAGGACGAGATGGACCGGGCACG
GGCCGCGCTGGACACGGTGTGCAGACACAACTACGGGGTGGAAGAGCTCA
CCACGTTGCAGCGGCGA
>dqbrw269new
GATTTCGTGTACCAGTGTAAGTGCGAGTGCTATTTCACCAACGGGACGGA
GCGGGTGCGGCTTCTGGCTAAATACATCTATAACCGGGAGGAGTTCGTGC
GCTTCGACAGCGACGTGGGGGAGTACCGGGCGGTCACGGAGCTCGGGCGG
CCCTTGGCTGAGTACTGGAACCCGCAGAAGGACAACATGGAGCAGGTACG
GGCCGAGCTGGACACGGTGTGCAGACACAACTACGGGTTGGAAGAGCTCT
ACACGTTGCAGCGGCGA
>dqbw30new
GATTTCGTGTACCAGTTTAAGGGCGAGTGCTATTTCACCAACGGGACGGA
GCGGGTGCGGCTTCTGACGAGAAACATCTATAACCGGGAGGAGTTCGTGC
GCTTCGACAGCGACGTGGGGGAGTTCCGGGCGGTCACGGAGCTCGGGCGG
CCCTGGGCTGAGTACTGGAACGGGCAGAAGGAGATCTTGGAGCGGAAGCG
GGCCGAGCTGGACAcGGTGTGCAGACACAACTACGGGAGGGAAGAGCTCA
CCACGTTGCAGCGGCGA
>DQB1*03801
GATTTCGTGTTCCAGTTTAAGTTCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTACCGGGCGGTCACGGAGCTCGGGCGG
CCCTGGGCTGAGTACTGGAACCCGCAGAAGGACGAGATGGACCGGGTACG
GGCCGAGCTGGACACGGTGTGCAGACACAACTACGGGTTGGAAGAGCTCA
CCACGTTGCAGCGGCGA
>DQB1*04001
GATTTCGTGTACCAGTTTAAGGGCGAGTGCTATTTCACCAACGGGACGGA
GCGGGTGCGGCTTCTGGCGAGAGACATCTATAACCGGGAGGAGCACGTGC
GCTTCGACAGCGACGTGGGGGAGTaCCGGGCGGTCACGGAGCTCGGGCGG
CCCTGGGCTGAGTACTGGAACGGGCAGAAGGAGATCTTGGAGCGGAAGCG
GGCCGAGCTGGACACGGTGTGCAGACACAACTACGGGGTGGAAGAGCTCT
ACACGTTGCAGCGGCGA
>dqb383-9
GATTTCGTGTtCCAGTTTAAGGGCGAGTGCTATTTCACCAACGGGACGGA
GCGGGTGCGGCTTCTGGcGAGAGACATCTATAACCGGGAGGAGCACGTGC
GCTTCGACAGCGACGTGGGGGAGTaCCGGGCGGTCACGGAGCTCGGGCGG
CCCTGGGCTGAGTACTGGAACGGGCAGAAGGAGATCTTGGAGCGGAAGCG
GGCCGAGCTGGACACGGTGTGCAGACACAACTACGGGGTGGAAGAGCTCT
ACACGTTGCAGCGGCGA
>dqb-a32-008v
GATTTCGTGTACCAGTTTAAGGGCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTtCCGGGCGGTCACGGAGCTCGGGCGG
CCCGACGCTGAGTACTGGAACCcgCAGAAGGACGAGATGGACCGGGTACG
GGCCGAGCTGGACACGGTGTGCAGACACAACTACGGGAGGGAAGAGCTCA
CCACGTTGCAGCGGCGA
>dqbwAnew
GATTTCGTGTACCAGTGTAAGGGCGAGTGCTATTTCACCAACGGGACGGA
GCGGGTGCGGCTTCTGACGAGAGGCATCTATAACCGGGAGGAGCACGTGC
GCTTCGACAGCGACGTGGGGGAGTACCGGGCGGTCACGGAGCTCGGGCGG
CCCTGGGCTGAGTACTGGAACGGGCAGAAGGAGATCTTGGAGCGGAAGCG
GGCCGAGGTGGACACGGTGTGCAGACACAACTACGGGAGGGAAGAGCTCA
CCACGTTGCAGCGGCGA
>DQB1*04101
GATTTCGTGTTCCAGTTTAAGGGCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCTGGGCTGAGTACTGGAACCCGCAGAAGGACGAGATGGACCGGGTACG
GGCCGAGCTGGACACGGTGTGCAGACACAACTACGGGTTGGAAGAGCTCT
ACACGTTGCAGCGGCGA
>DQB1*04201
GATTTCGTGTTCCAGTTTAAGTTCGAGTGCTATTTCACCAACGGGACGGA
GCGGGTGCGGCTTCTGGCTAAATACATCTATAACCGGGAGGAGTTCGTGC
GCTTCGACAGCGACGTGGGGGAGTACCGGGCGGTCACGGAGCTCGGGCGG
CCAGACGCTGAGTACTGGAACCCGCAGAAGGACGAGATGGACCGGGTACG
GGCCGAGCTGGACACGGTGTGCAGACACAACTACGGGGTGGAAGAGCTCT
ACACGTTGCAGCGGCGA
>dqb381-9
GATTTCGTGTTCCAGTTTAAGTTCGAGTGCTATTTCACCAACGGGACGGA
GCGGGTGCGGCTTCTGgCTAgATACATCTATAACCGGGAGGAGTTCGTGC
GCTTCGACAGCGACGTGGGGGAGtACCGGGCGGTCACGGAGCTCGGGCGG
CCCtACGCTGAGTACTGGAACCGACAGAAGGACaAGATGGACCGGGTACG
GGCCGAGCTGGACACGGTGTGCAGACACAACTACGGGAGGGAAGAGCTCA
CCACGTTGCAGCGGCGA
>DQB1*04301
GATTTCGTGTaCCAGTTTAAGGgCGAGTGCTATTTCACCAACGGGACGGA
GCGGGTGCGGCTTCTGgCtAaAtACATCTATAACCGGGAGGAGttCGTGC
GCTTCGACAGCGACGTGGGGGAGTACCGGGCGGTCACGGAGCTCGGGCGG
CCCGACGCTGAGTACTGGAACGGGCAGAAGGAGaTCTTGGAGCGGAAGCG
GGCCGAGCTGGACACGGTGTGCAGACACAACTACGGGgtGGAAGAGCTCt
aCACGTTGCAGCGGCGA
>DQB/AA
GATTTCGTGTTCCAGTTTAAGGGCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTACCGGGCGGTCACGGAGCTCGGGCGG
CCCTGGGCTGAGTACTGGAACCCGCAGAAGGACAACATGGACCGGGTACG
GGCCGAGCTGGACACGGTGTGCAGACACAACTACGGGTTGGAAGAGCTCT
ACACGTTGCAGCGGCGA
>DQB/BB
GATTTCGTGTTCCAGTTTAAGGGCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTACCGGGCGGTCACGGAGCTCGGGCGG
CCCTGGGCTGAGTACTGGAACCCGCAGAAGGACAAGATGGACCGGGTACG
GGCCGAGCTGGACACGGTGTGCAGACACAACTACGGGTTGGAAGAGCTCT
ACACGTTGCAGCGGCGA
>DQB/DD
GATTTCGTGTTCCAGTTTAAGGCCGAGTGCTATTTCACCAACGGGACGGA
GCGGGTGCGGCTTCTGACGAGAGACATCTATAACCGGGAGGAGCACGTGC
GCTTCGACAGCGACGTGGGGGAGTACCGGGCGGTCACGGAGCTCGGGCGG
CCCTACGCTGAGTACTGGAACCCGCAGAAGGAGTTCTTGGAGCGGGCGCG
GGCCGAGGTGGACACGGTGTGCAGACACAACTACGGGGTGGAAGAGCTCA
CCACGTTGCAGCGGCGA
>DQB1*04401
GATTTCGTGTTCCAGTTTAAGGCCGAGTGCTATTTCACCAACGGGACGGA
GCGGGTGCGGCTTCTGACGAGAGACATCTATAACCGGGAGGAGCACGTGC
GCTTCGACAGCGACGTGGGGGAGTACCGGGCGGTCACGGAGCTCGGGCGG
CCCGACGCTGAGTACTGGAACGGGCAGAAGGAGTTCTTGGAGCGGGCGCG
GGCCGCGGTGGACACGGTGTGCAGACACAACTACGGGGTGGAAGAGCTCA
CCACGTTGCAGCGGCGA
>DQB/H
GATTTCGTGTTCCAGTTTAAGGCCCAGTGCTATTTCACCAACGGGACGGA
GCGGGTGCGGCTTCTGACGAGAGACATCTATAACCGGGAGGAGCACGTGC
GCTTCGACAGCGACGTGGGGGAGTACCGGGCGGTCACGGAGCTCGGGCGG
CCCGACGCTGAGTACTGGAACGGGCAGAAGGAGTTCTTGGAGCGGGCGCG
GGCCGCGGTGGACACGGTGTGCAGACACAACTACGGGGTGGAAGAGCTCA
CCACGTTGCAGCGGCGA
>DQB/I
GATTTCGTGTTCCAGTTTAAGGCCGAGTGCTATTTCACCAACGGGACGGA
GCGGGTGCGGCTTCTGACGAGAGACATCTATAACCGGGAGGAGCACGTGC
GCTTCGACAGCGACGTGGGGGAGTACCGGGCGGTCACGGAGCTCGGGCGG
CCCTACGCTGAGTACTGGAACGGGCAGAAGGAGTTCTTGGAGCGGGCGCG
GGCCGAGGTGGACACGGTGTGCAGACACAACTACGGGGTGGAAGAGCTCA
CCACGTTGCAGCGGCGA
>DQB/J
GATTTCGTGTACCAGTTTAAGGGCGAGTGCTATTTCACCAACGGGACGGA
GCGGGTGCGGCTTCTGGCGAGAGACATCTATAACCGGGAGGAGTTCGTGC
GCTTCGACAGCGACGTGGGGGAGTACCGGGCGGTCACGGAGCTCGGGCGG
CCCTGGGCTGAGTACTGGAACGGGCAGAAGGAGATCTTGGAGCGGAAGCG
GGCCGAGCTGGACACGGTGTGCAGACACAACTACGGGGTGGAAGAGCTCA
CCACGTTGCAGCGGCGA
>DQB1*04501
GATTTCGTGTtCCAGTTTAAGGCCGAGTGCTATTTCACCAACGGGACGGA
GCGGGTGCGGCTTCTGaCGAGAGACATCTATAACCGGGAGGAGCACGTGC
GCTTCGACAGCGACGTGGGGGAGTACCGGGCGGTCACGGAGCTCGGGCGG
CCCTaCGCTGAGTACTGGAACGGGCAGAAGGAGtTCTTGGAGCGGgCGCG
GGCcGCGgTGGAcAcGGTGTGcAGACAcAACTACGGGGTGGAAGAGCTCa
cCACGTTGCAGCGGCGA
>DQB/R
gATTTcGTGTACCAGTTTAAGTTCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACccGCAGAAGGAcCagaTGGACCgGgtaCG
GGCCGAGCTGGACACGGTGTGCAGACACAACTACgGGgTGGAAGAGCTCT
ACACGTTGCAGCGGCGA
>DQB/S
GATTTCGTGTtCCAGTGTAAGGgCGAGTGCTATTTCACCAACGGGACGGA
GCGGGTGCGGcTTCTGaCTAAATACATCTATAACCGGGAGGAGTaCGTGC
GCTTCGACAGCGACGTGGGGGAGTaCCGGGCGGTCACGGAGCTCGGGCGG
CCCtggGCTGAGTACTGGAACcCGCAGAAGGAcCagaTGGAcCgGGtaCG
GGCcgaGcTGGACACGGTGTGCAGACAcAACTACGGGtTGGAAGAGCTCT
ACACGTTGCAGCGGCGA
>DQB/U
GATTtCGTGTACCAGTTTAAGGGCGAGTGCTATTTCACCAACGGGACGGA
GCGGGTGCGGCTTCTGGCGAGAGACATCTATAACCGGGAGGAGCACGTGC
GCTTCGACAGCGACGTGGGGGAGTACCGGGCGGTCACGGAGCTCGGGCGG
CCCgACGCTGAGTACTGGAACGGGCAGAAGGAGTTCTTGGAGCGGGCGCG
GGCCGCGGTGGACACGGTGTGCAGACACAACTACGGGGTGGAAGAGCTCA
CCACGTTGCAGCGGCGA
>DQB/CVA307/B
GATTTCGTGTwCCAGTTTAAGGGCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTACCGGGCGGTCACGGAGCTCGGGCGG
CCCTGGGCTGAGTACTGGAACCCGCAgAAGgACGAGATGGACcGGGTACg
GGCCGAGCTGGACACGGTGTGCAGACACAACTACGGGgTGGAAGAGCTCT
ACACGTTGCAGCGGCGA
>dqbIW001
GATTTCGTGTTCCAGTTTAAGTTCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCCGCAGAAGGACGAGATGGACCGGGTACG
GGCCGAGCTGGACACGGTGTGCAGACACAACTACGGGGTGGAAGAGCTCT
ACACGTTGCAGCGGCGA
>dqb1*03602
GATTTCGTGTTCCAGTATAAGGCCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCCGCAGAAGGACGAGATGGACCGGGTACG
GGCCGAGCTGGACACGGTGTGCAGACACAACTACGGGGTGGAAGAGCTCT
ACACGTTGCAGCGGCGA
>dqb1*03603
GATTTCGTGTTCCAGTATAAGGCCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCGACGCTGAGTACTGGAACCCGCAGAAGGACGAGATGGACCGGGTACG
GGCCGAGCTGGACACGGTGTGCAGACACAACTACGGGAGGGAAGAGCTCA
CCACGTTGCAGCGGCGA
>dqb1*00202
GATTTCGTGTTCCAGTATAAGGCCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTTCCGGGCGGTCACGGAGCTCGGGCGG
CCCGACGCTGAGTACTGGAACCGACAGAAGGACGAGATGGACCGGGTACG
GGCCGAGCTGGACACGGTGTGCAGACACAACTACGGGGTGGAAGAGCTCT
ACACGTTGCAGCGGCGA
>dqb1*04601
GATTTCGTGTACCAGTTTAAGTTCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTTCCGGGCGGTCACGGAGCTCGGGCGG
CCCGACGCTGAGTACTGGAACCGACAGAAGGACGAGATGGACCGGGTACG
GGCCGAGCTGGACACGGTGTGCAGACACAACTACGGGAGGGAAGAGCTCA
CCACGTTGCAGCGGCGA
>DQB1*04701
GATTTCGTGTTCCAGTGTAAGTTCGAGTGCTATTTCACCAACGGGACGGA
GCGGGTGCGGTTTCTGGCTAAATACATCTATAACCGGGAGGAGTTCGTGC
GCTTCGACAGCGACGTGGGGGAGTACCGGGCGGTCACGGAGCTCGGGCGG
CCCGACGCTGAGTCCTGGAACGGGCAGAAGGAGCTCTTGGAGCAGGAGCG
GGCAACGGTGGACACGGTGTGCAGACACAACTACGGGGTGGAAGAGCTCT
ACACGTTGCAGCGGCGA
>lkawd14
gATTtCGTgTaCcAGTTTAAGGGCGAGTGCTATTTCACCAACGGGACGGA
GCGGGTGCGGCTTCTGACTAAACACATCTATAACCGGGAGGAGTTCGTGC
GCTTCGACAGCGACGTGGGGGAGTTCCGGGCGGTCACGGAGCTCGGGCGG
CCCGACGCTGAGTACTGGAACCGGCAGAAGGACGAGGTGGACCGGGTACG
GGCCGAGGTGGACACGGTGTGCAGACACAACTACGGGATGGAGGAGCTCA
CCACGTTGCAGCGGCGA
>lk-awd16
gATTtCgTGTaCcAGTTTAaGGGCGAGTGCTATTTCACCAACGGGACGGA
GCGGGTGCGGTTCGTGGACAGATACATCTATAACCGGGAGGAGTTCGTGC
GCTTCGACAGCGACGTGGGGGAGTTCCGGGCGGTCACGGAGCTCGGGCGG
CCCGACGCTGAGTACTGGAACCGGCAGAAGGACGAGGTGGACCGGGTACG
GGCCGAGGTGGACACGGTGTGCAGACACAACTACGGGATGGAGGAGCTCA
CCACGTTGCAgCGGCGA
>dqb013 + 017
GATTTCGTGTWCCAGTkTAAGkyCGAGTGCTATTTCACCAACGGGACGGA
GCGGGTGCGGyTTCTGrCTAAATACATCTATAACCGGGAGGAGTTCGTGC
GCTTCGACAGCGACGTGGGGGAGTACCGGGCGGTCACGGAGCTCGGGCGG
CCCGACGCTGAGTmCTGGAACsSGCAGAAGGASkWSWTGGASCrGGWrCG
GGCmrmGSTGGACACGGTGTGCAGACACAACTACGGGGTGGAAGAGCTCT
ACACGTTGCAGCGGCGA
>dqb019 + 022
GATTTCGTGTwCCAGTkTAAGGsCGAGTGCTATTTCACCAACGGGACGGA
GCGGGTGCGGyTTCTGrCTAAATACATCTATAACCGGGAGGAGTTCGTGC
GCTTCGACAGCGACGTGGGGGAGTWCCGGGCGGTCACGGAGCTCGGGCGG
CCCTCGGCTGAGTACTGGAACSSGCAGAAGGASSWSWTGGASCrrGWrCG
GGCmrmGSTGGACACGGTGTGCAGACACAACTACGGGWkGGAAGAGCTCA
CCACGTTGCAGCGGCGA
>dqb8061new
GATTTCGTGTACCAGTGTAAGGCCGAGTGCTATTTCACCAACGGGACGGA
GCGGGTGCGGCTTCTGGCGAGAGACATCTATAACCGGGAGGAGCACGTGC
GCTTCGACAGCGACGTGGGGGAGTTCCGGGCGGTCACGGAGCTCGGGCGG
CCCTCGGCTGAGTACTGGAACGGGCAGAAGGAGCTCTTGGAGCAGAGGCG
GGCCGAGCTGGACACGGTGTGCAGACACAACTACGGGAGGGAAGAGCTCA
CCACGTTGCAGCGGCGA
>dqb8062new
GATTTCGTGTACCAGTGTAAGGGCGAGTGCTATTTCACCAACGGGACGGA
GCGGGTGCGGCTTCTGGCGAGAGACATCTATAACCGGGAGGAGCACGTGC
GCTTCGACAGCGACGTGGGGGAGTACCGGGCGGTCACGGAGCTCGGGCGG
CCCTACGCTGAGTACTGGACGGGCAGAAGGAAGCTCTTGGAGCGGAAGCG
GGCCGAGCTGGACACGGTGTGCAGACACAACTACGGGTTGGAAGAGCTCA
CCACGTTGCAGCGGCGA
>dqb-1k-ewC
GATTTCGTGTTCCAGTTTAAGGGCGAGTGCTATTTCACCAACGGGACGGA
GCGGGTGCGGCTTCTGACTAAATACATCTATAACCGGGAGGAGTTCGTGC
GCTTCGACAGCGACGTGGGGGAGTACCGGGCGGTCACGGAGCTCGGGCGG
CCCgaCGCTGAGTACTGGAACCCGCAGAAGGACGAGATGGACCGGGTACG
GGCCGAGCTGGACACGGTGTGCAGACACAACTACGGGTTGGAAGAGCTCT
ACACGTTGCAGCGGCGA
>dqb-1k-ew88
GATTTCGTGTtCCAGTTTAAGGGCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTtCCGGGCGGTCACGGAGCTCGGGCGG
CCCgaCGCTGAGTACTGGAACCCGCAGAAGGACGAGATGGACCGGGTACG
GGCCGAGCTGGACACGGTGTGCAGACACAACTACGGGTTGGAAGAGCTCt
aCACGTTGCAGCGGCGA
>dqb-1k-023v
GATTTCGTGTACCAGTTTAASGGCGAGTGCTATTTCACCAACGGGACGGA
GCGGGTGCGGCTTCTGACTAAATACATCTATAACCGGGAGGAGTACGTGC
GCTTCGACAGCGACGTGGGGGAGTtCCGGGCGGTCACGGAGCTCGGGCGG
CCCTCGGCTGAGTACTGGAACCCGCAGAAGGACGAGATGGACCGGGTACG
GGCCGAGCTGGACACGGTGTGCAGACACAACTACGGGTTGGAAGAGCTCA
CCACGTTGCAGCGGCGA
DLA-DRB
>DRB1*00101
CACATTTCTTGGAGGTGGCAAAGTCCGAGTGCTATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGTCGCTGAGTCCTGGAACGGGCAGAAGGAGATCTTGGAGCAGGAG
CGGGCAACGGTGGACACCTACTGCAGACACAACTACGGGGTGATTGAGAG
CTTCACGGTGCAGCGGCGAG
>DRB1*00102
CACATTTCTTGGAGGTGGCAAAGTCCGAGTGCTATTTCACCAACGGGACG
GAGCGGGTGCGGTTCGTGGAAAGATACATCCATAACCGGGAGGAGTTCGT
GCGCTTCGACAGCGACGTGGGGGAGTTCCGGGCGGTCACGGAGCTCGGGC
GGCCCGTCGCTGAGTCCTGGAACGGGCAGAAGGAGATCTTGGAGCAGGAG
CGGGCAACGGTGGACACCTACTGCAGACACAACTACGGGGTGATTGAGAG
CTTCACGGTGCAGCGGCGAG
>DRB1*00201
CACATTTCTTGGAGATGGTAAAGTTCGAGTGCCATTTCACCAACGGGACG
GAGCGGGTGCGGTATCTGGCGAGAGACATCTATAACCGGGAGGAGATCCT
GCGCTTCGACAGCGACGTGGGGGAGTACCGGGCGGTCACGGAGCTCGGGC
GGCCCATCGCTGAGTCCTGGAACCGGCAGAAGGAGATCTTGGAGCAGAGG
CGGGCCGCGGTGGACACCTACTGCAGACACAACTACGGGGTGATTGAGAG
CTTCGCGGTGCAGCGGCGAG
>DRB1*00202
CACATTTCTTGGAGATGGTAAAGTTCGAGTGCCATTTCACCAACGGGACG
GAGCGGGTGCGGTATCTGGCGAGAGACATCTATAACCGGGAGGAGATCCT
GCGCTTCGACAGCGACGTGGGGGAGTACCGGGCGGTCACGGAGCTCGGGC
GGCCCATCGCTGAGTCCTGGAACCGGCAGAAGGAGATCTTGGAGCAGAGG
CGGGCCGCGGTGGACACCTACTGCAGACACAACTACGGGGTGATTGAGAG
CTTCACGGTGCAGCGGCGAG
>DRB1*00301
CACATTTCTTGGAGGTGGCAAAGTCCGAGTGCTATTTCACCAACGGGACG
GAGCGGGTGCGGTTCGTGGAAAGATACATCCATAACCGGGAGGAGAACGT
GCGCTTCGACAGCGACGTGGGGGAGTTCCGGGCGGTCACGGAGCTCGGGC
GGCCCGACGCTGAGTCCTGGAACCGGCAGAAGGAGCTCTTGGAGCGGAAG
CGGGCCGCGGTGGACACCTACTGCAGACACAACTACGGGGTGATTGAGAG
CTTCACGGTGCAGCGGCGAG
>DRB1*00401
CACATTTCGTGTACCAGTTTAAGCCCGAGTGCCATTTCACCAACGGGACG
GAGCGGGTGCGGTTCGTGGAAAGACACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACGGGCAGAAGGAGCTCTTGGAGCAGGAG
CGGGCAACGGTGGACACCTACTGCAGACACAACTACGGGGTGATTGAGAG
CTTCACGGTGCAGCGGCGAG
>DRB1*00501
CACATTTCTTGGAGATGTTAAAGTTCGAGTGCCATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGGAG
CGGGCCGCGGTGGACACCTACTGCAGACACAACTACCGGGTGGGCGAGAG
CTTCACGGTGCAGCGGCGAG
>DRB1*00601
CACATTTCTTGGAGGTGGCAAAGGCCGAGTGCCATTTCACCAACGGGACG
GAGCGGGTGCGGTTCGTGGAAAGATACATCTATAACCGGGAGGAGTACGT
GCGCTTCGACAGCGACGTGGGGGAGTACCGGGCGGTCACGGAGCTCGGGC
GGCCCGACGCTGAGTACTGGAACCCGCAGAAGGAGCTCTTGGAGCGGGCG
CGGGCCGCGGTGGACACCTACTGCAGACACAACTACGGGGTGGGCGAGAG
CTTCACGGTGCAGCGGCGAG
>DRB1*00701
CACATTTCTTGGAGGTGGCAAAGGCCGAGTGCCATTTCACCAACGGGACG
GAGCGGGTGCGGTTCGTGGAAAGATACATCTATAACCGGGAGGAGTTCGT
GCGCTTCGACAGCGACGTGGGGGAGTACCGGGCGGTCACGGAGCTCGGGC
GGCCCGACGCTGAGTACTGGAACCCGCAGAAGGAGCTCTTGGAGGGGGGC
CGGGCCGCGGTGGACACCTACTGCAGACACAACTACGGGGTGGGCGAGAG
CTTCACGGTGCAGCGGCGAG
>DRB1*00801
CACATTTCGTGAAGATGTATAAGGCCGAGTGCCATTTCACCAACGGGACG
GAGCGGGTGCGGTATCTGATGAGAGACATCTATAACCGGGAGGAGTTCGT
GCGCTTCGACAGCGACGTGGGGGAGTTCCGGGCGGTCACGGAGCTCGGGC
GGCGCGACGCTGAGTACTGGAACGGGCAGAAGGAGATCTTGGAGCGGAAG
CGGGCCGCGGTGGACACCTACTGCAGACACAACTACCGGGTGGGCGAGAG
CTTCACGGTGCAGCGGCGAG
>DRB1*00802
CACATTTCGTGAAGATGTATAAGGCCGAGTGCCATTTCACCAACGGGACG
GAGCGGGTGCGGTATCTGATGAGAGACATCTATAACCGGGAGGAGTTCGT
GCGCTTCGACAGCGACGTGGGGGAGTTCCGGGCGGTCACGGAGCTCGGGC
GGCGCGACGCTGAGTACTGGAACGGGCAGAAGGAGATCTTGGAGCGGAAG
CGGGCCGCGGTGGACACCTACTGCAGACACAACTACGGGGTGATTGAGAG
CTTCGCGGTGCAGCGGCGAG
>DRB1*00901
CACATTTCTTGGAGGTGGCAAAGTCCGAGTGCTATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCCCGACGCTGAGTCCTGGAACCGGCAGAAGGAGTTCTTGGAGCGGAAG
CGGGCCGCGGTGGACACCTACTGCAGACACAACTACGGGGTGATTGAGAG
CTTCACGGTGCAGCGGCGAG
>DRB1*010011
CACATTTCGTGTACCAGTTTAAGCCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGTCGCTGAGTCCTGGAACGGGCAGAAGGAGCTCTTGGAGCAGGAG
CGGGCAACGGTGGACACCTACTGCAGACACAACTACGGGGTGATTGAGAG
CTTCaCGGTGCAGCGGCGAG
>DRB1*010012
CACATTTCGTGTACCAGTTTAAGCCCGAGTGCCATTTCACCAACGGGACG
GAGCGGGTGCGGTTCGTGGAAAGATACATCCACAACCGGGAGGAGTTCGT
GCGCTTCGACAGCGACGTGGGGGAGTACCGGGCGGTCACGGAGCTCGGGC
GGCCCGTCGCTGAGTCCTGGAACGGGCAGAAGGAGCTCTTGGAGCAGGAG
CGGGCAACGGTGGACACCTACTGCAGACACAACTACGGGGTGATTGAGAG
CTTCRCGGTGCAGCGGCGAG
>DRB1*01101
CACATTTCGTGAAGATGTTTAAGGCCGAGTGCCATTTCACCAACGGGACG
GAGCGGGTGCGGCTTCTGGCGAGAAGCATCTATAACCGGGAGGAGTTCGT
GCGCTTCGACAGCGACGTGGGGGAGTTCCGGGCGGTCACGGAGCTCGGGC
GGCGCGACGCTGAGTCCTGGAACCGGCAGAAGGAGCTCTTGGAGCAGAGG
CGGGCCGCGGTGGACACCTACTGCAGACACAACTACCGGGTGGGCGAGAG
CTTCACGGTGCAGCGGCGAG
>DRB1*01201
CACATTTCGTGAGGATGTATAAGGCCGAGTGCCATTTCACCAACGGGACG
GAGCGGGTGCGGTTTCTGGCGAGAAGCATCTATAACCGGGAGGAGTTCGC
GCGCTTCGACAGCGACGTGGGGGAGTACCGGGCGGTCACGGAGCTCGGGC
GGCGCGACGCTGAGTCCTGGAACCGGCAGAAGGAGCTCTTGGAGCGGAGG
CGGGCCGAGGTGGACACCTACTGCAGACACAACTACGGGGTGATTGAGAG
CTTCACGGTGCAGCGGCGAG
>DRB1*01301
CACATTTCGTGTACCAGTTTAAGCCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGGAG
CGGGCCGCGGTGGACACCTACTGCAGACACAACTACCGGGTGGGCGAGAG
CTTCACGGTGCAGCGGCGAG
>DRB1*01302
CACATTTCGTGTACCAGTTTAAGCCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGGAG
CGGGCCGCGGTGGACACGGTGTGCAGACACAACTACCGGGTGGGCGAGAG
CTTCACGGTGCAGCGGCGAG
>DRB1*01401
CACATTTCTTGGAGATGTTAAAGTCCGAGTGCTATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCCCGACGCTGAGTACTGGAACCCGCAGAAGGAGCTCTTGGAGCGGGCG
CGGGCCGCGGTGGACACCTACTGCAGACACAACTACGGGGTGGGCGAGAG
CTTCACGGTGCAGCGGCGAG
>DRB1*01501
CACATTTCTTGGAGATGGTAAAGTTCGAGTGCCATTTCACCAACGGGACG
GAGCGGGTGCGGCTTCTGGTGAGAGACATCTATAACCGGGAGGAGCACGT
GCGCTTCGACAGCGACGTGGGGGAGTACCGGGCGGTCACGGAGCTCGGGC
GGCCCGACGCTGAGTACTGGAACGGGCAGAAGGAGCTCTTGGAGCAGAGG
CGGGCCGAGGTGGACACGGTGTGCAGACACAACTACGGGGTGATTGAGAG
CTTCACGGTGCAGCGGCGAG
>DRB1*01502
CACATTTCTTGGAGATGGTAAAGTTCGAGTGCCATTTCACCAACGGGACG
GAGCGGGTGCGGCTTCTGGTGAGAGACATCTATAACCGGGAGGAGCACGT
GCGCTTCGACAGCGACGTGGGGGAGTACCGGGCGGTCACGGAGCTCGGGC
GGCCCGACGCTGAGTACTGGAACGGGCAGAAGGAGCTCTTGGAGCAGAGG
CGGGCCGAGGTGGACACGGTGTGCAGACACAACTACGGGGTGATTGAGAG
CTTCGCGGTGCAGCGGCGAG
>DRB1*01503
CACATTTCTTGGAGATGGTAAAGTTCGAGTGCCATTTCACCAACGGGACG
GAGCGGGTGCGGCTTCTGGTGAGAGACATCTATAACCGGGAGGAGCACGT
GCGCTTCGACAGCGACGTGGGGGAGTACCGGGCGGTCACGGAGCTCGGGC
GGCCCGACGCTGAGTACTGGAACGGGCAGAAGGAGCTCTTGGAGCAGAGG
CGGGCCGAGGTGGACACCTACTGCAGACACAACTACGGGGTGATTGAGAG
CTTCACGGTGCAGCGGCGAG
>DRB1*01504
CACATTTCTTGGAGATGGTAAAGTTCGAGTGCCATTTCACCAACGGGACG
GAGCGGGTGCGGCTTCTGGTGAGAGACATCTATAACCGGGAGGAGCACGT
GCGCTTCGACAGCGACGTGGGGGAGTTCCGGGCGGTCACGGAGCTCGGGC
GGCCCGACGCTGAGTACTGGAACGGGCAGAAGGAGCTCTTGGAGCAGAGG
CGGGCCGAGGTGGACACGGTGTGCAGACACAACTACGGGGTGATTGAGAG
CTTCACGGTGCAGCGGCGAG
>DRB1*01601
CACATTTCTTGGAGGTGGCAAAGGCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCGGAAG
CGGGCCGAGGTGGACACCTACTGCAGACACAACTACGGGGTGATTGAGAG
CTTCACGGTGCAGCGGCGAG
>DRB1*01701
CACATTTCGTGAAGATGTTTAAGGCCGAGTGCCATTTCACCAACGGGACG
GAGCGGGTGCGGTTTCTGGCGAGAAGCATCTATAACCGGGAGGAGTTCGT
GCGCTTCGACAGCGACGTGGGGGAGTACCGGGCGGTCACGGAGCTCGGGC
GGCGCGACGCTGAGTCCTGGAACCGGCAGAAGGAGCTCTTGGAGCGGGCG
CGGGCCGCGGTGGACACCTACTGCAGACACAACTACGGGGTGATTGAGAG
CTTCACGGTGCAGCGGCGAG
>DRB1*01702
CACATTTCGTGAAGATGTTTAAGGCCGAGTGCCATTTCACCAACGGGACG
GAGCGGGTGCGGTTTCTGGCGAGAAGCATCTATAACCGGGAGGAGTTCGT
GCGCTTCGACAGCGACGTGGGGGAGTACCGGGCGGTCACGGAGCTCGGGC
GGCGCGACGCTGAGTCCTGGAACCGGCAGAAGGAGCTCTTGGAGCGGGCG
CGGGCCGCGGTGGACACCTACTGCAGACACAACTACCGGGTGGGCGAGAG
CTTCACGGTGCAGCGGCGAG
>DRB1*01801
CACATTTCTTGGAGGTGGCAAAGTCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCGGAAG
CGGGCCGAGGTGGACACCTACTGCAGACACAACTACGGGGTGATTGAGAG
CTTCACGGTGCAGCGGCGAG
>DRB1*01901
CACATTTCGTGAGGATGTATAAGGCCGAGTGCCATTTCACCAACGGGACG
GAGCGGGTGCGGTTTCTGGCGAGAAGCATCTATAACCGGGAGGAGTTCGT
GCGCTTCGACAGCGACGTGGGGGAGTACCGGGCGGTCACGGAGCTCGGGC
GGCGCGACGCTGAGTCCTGGAACCGGCAGAAGGAGCTCTTGGAGCGGAGG
CGGGCCGAGGTGGACACCTACTGCAGACACAACTACGGGGTGATTGAGAG
CTTCACGGTGCAGCGGCGAG
>DRB1*02001
CACATTTCTTGAAGATGGTAAAGTTCGAGTGCCATTTCACCAACGGGACG
GAGCGGGTGCGGTTGGTGGAAAGAGACATCTATAACCGGGAGGAGTACGT
GCGCTTCGACAGCGACGTGGGGGAGTACCGGGCGGTCACGGAGCTCGGGC
GGCCCTCGGCTGAGTCCTGGAACCGGCAGAAGGAGTTCTTGGAGCAGAGG
CGGGCCGAGGTGGACACGGTGTGCAGACACAACTACGGGGTGGGCGAGAG
CTTCACGGTGCAGCGGCGAG
>DRB1*02101
CACATTTCGTGAAGATGTTTAAGGCCGAGTGCCATTTCACCAACGGGACG
GAGCGGGTGCGGCTTCTGGCGAGAAGCATCTATAACCGGGAGGAGTTCGT
GCGCTTCGACAGCGACGTGGGGGAGTTCCGGGCGGTCACGGAGCTCGGGC
GGCGCGACGCTGAGTCCTGGAACCGGCAGAAGGAGCTCTTGGAGCAGAGG
CGGCCCGCGGTGGACACCTACTGCAGACACAACTACCGGGTGGGCGAGAG
CTTCACGGTGCAGCGGCGAG
>DRB1*02201
CACATTTCGTGTACCAGTTTAAGCCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACGGGCAGAAGGAGATCTTGGAGCAGGAG
CGGGCCGAGGTGGACACCTACTGCAGACACAACTACGGGGTGATTGAGAG
CTTCACGGTGCAGCGGCGAG
>DRB1*02301
CACATTTCTTGGAGATGTTAAAGTTCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGGAG
CGGGCCGCGGTGGACACCTACTGCAGACACAACTACCGGGTGGGCGAGAG
CTTCACGGTGCAGCGGCGAG
>DRB1*02401
CACATTTCTTGGAGGTGTTAAAGTCCGAGTGCTATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCGCGACGCTGAGTCCTGGAACCGGCAGAAGGAGCTCTTGGAGCGGAAG
CGGGCCGAGGTGGACACCTACTGCAGACACAACTACCGGGTGGGCGAGAG
CTTCACGGTGCAGCGGCGAG
>DRB1*02501
CACATTTCTTGGAGGTGTTAAAGTTCGAGTGCCATTTCACCAACGGGACG
GAGCGGGTGCGGTTGGTGGAAAGATACATCTATAACCGGGAGGAGTTCGC
GCGCTTCGACAGCGACGTGGGGGAGTACCGGGCGGTCACGGAGCTCGGGC
GGCGCGACGCTGAGTCCTGGAACCGGCAGAAGGAGCTCTTGGAGCAGAGG
CGGGCCGCGGTGGACACCTACTGCAGACACAACTACCGGGTGGGCGAGAG
CTTCACGGTGCAGCGGCGAG
>DRB1*02601
CACATTTCTTGGAGATGTTAAAGTCCGAGTGCTATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCCCGACGCTGAGTACTGGAACCGGCAGAAGGAGCTCTTGGAGCGGAAG
CGGGCCGAGGTGGACACCTACTGCAGACACAACTACGGGGTGATTGAGAG
CTTCACGGTGCAGCGGCGAG
>DRB1*02701
CACATTTCGTGTACCAGTTTAAGGCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCGCGACGCTGAGTCCTGGAACGGGCAGAAGGAGATCTTGGAGCAGGAG
CGGGCAACGGTGGACACCTACTGCAGACACAACTACGGGGTGATTGAGAG
CTTCACGGTGCAGCGGCGAG
>DRB1*02801
CACATTTCTTGGAGGTGGCAAAGGCCGAGTGCCATTTCACCAACGGGACG
GAGCGGGTGCGGTTCGTGGAAAGATACATCTATAACCGGGAGGAGTTCGT
GCGCTTCGACAGCGACGTGGGGGAGTACCGGGCGGTCACGGAGCTCGGGC
GGCCCGACGCTGAGTACTGGAACCCGCAGAAGGAGCTCTTGGAGCGGGCG
CGGGCCGCGGTGGACACCTACTGCAGACACAACTACGGGGTGGGCGAGAG
CTTCACGGTGCAGCGGCGAG
>DRB1*02901
CACATTTCGTGAAGATGTATAAGGCCGAGTGCCATTTCACCAACGGGACG
GAGCGGGTGCGGTATCTGATGAGAGACATCTATAACCGGGAGGAGAACGT
GCGCTTCGACAGCGACGTGGGGGAGTTCCGGGCGGTCACGGAGCTCGGGC
GGCGCGACGCTGAGTCCTGGAACGGGCAGAAGGAGATCTTGGAGCGGAAG
CGGGCCGCGGTGGACACCTACTGCAGACACAACTACGGGGTGATTGAGAG
CTTCGCGGTGCAGCGGCGAG
>DRB1*03001
CACATTTCTTGGAGATGGTAAAGTTCGAGTGCCATTTCACCAACGGGACG
GAGCGGGTGCGGCTTCTGGTGAGAGACATCTATAACCGGGAGGAGCACGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGAGG
CGGGCCGAGGTGGACACGGTGTGCAGACACAACTACGGGGTGATTGAGAG
CTTCRCGGTGCAGCGGCGAG
>DRB1*03101
CACATTTCTTGAAGATGGTAAAGTTCGAGTGCCATTTCACCAACGGGACG
GAGCGGGTGCGGTATCTGATGAGAGACATCTATAACCGGGAGGAGTTCGT
GCGCTTCGACAGCGACGTGGGGGAGTTCCGGGCGGTCACGGAGCTCGGGC
GGCGCGACGCTGAGTCCTGGAACGGGCAGAAGGAGCTCTTGGAGCAGAAG
CGGGCCGCGGTGGACACCTACTGCAGACACAACTACGGGGTGATTGAGAG
CTTCGCGGTGCAGCGGCGAG
>DRB1*03201
CACATTTCGTGTACCAGTTTAAGCCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGTCGCTGAGTCCTGGAACGGGCAGAAGGAGATCTTGGAGCAGGAG
CGGGCAACGGTGGACACGGTGTGCAGACACAACTACGGGGTGATTGAGAG
CTTCACGGTGCAGCGGCGAG
>DRB1*03202
CACATTTCGTGTACCAGTTTAAGCCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGTCGCTGAGTCCTGGAACGGGCAGAAGGAGATCTTGGAGCAGGAG
CGGGCAACGGTGGACACCTACTGCAGACACAACTACGGGGTGATTGAGAG
CTTCACGGTGCAGCGGCGAG
>DRB1*03301
CACATTTCTTGGAGATGTTAAAGTTCGAGTGCCATTTCACCAACGGGACG
GAGCGGGTGCGGTATCTGGTGAGAGACATCTATAACCGGGAGGAGCACGT
GCGCTTCGACAGCGACGTGGGGGAGTACCGGGCGGTCACGGAGCTCGGGC
GGCCCGACGCTGAGTACTGGAACCGGCAGAAGGAGCTCTTGGAGCGGAGG
CGGGCCGAGGTGGACACCTACTGCAGACACAACTACCGGGTGGGCGAGAG
CTTCACGGTGCAGCGGCGAG
>DRB1*03501
CACATTTCGTGAAGATGTTTAAGGCCGAGTGCCATTTCACCAACGGGACG
GAGCGGGTGCGGCTTCTGGCGAGAAGCATCTATAACCGGGAGGAGTTCGT
GCGCTTCGACAGCGACGTGGGGGAGTTCCGGGCGGTCACGGAGCTCGGGC
GGCGCGACGCTGAGTCCTGGAACCGGCAGAAGGAGTTCTTGGAGCAGAGG
CGGGCCGCGGTGGACACCTACTGCAGACACAACTACCGGGTGGGCGAGAG
CTCACGGTGCAGCGGCGAG
>DRB1*03601
CACATTTCTTGGAGATGTTAAAGTCCGAGTGCCATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCGCGACGCTGAGTCCTGGAACCGGCAGAAGGAGCTCTTGGAGCGGAAG
CGGGCCGAGGTGGACACCTACTGCAGACACAACTACGGGGTGATTGAGAG
CTTCACGGTGCAGCGGCGAG
>DRB1*03701
CACATTTCTTGgAGgTGGcAAAGgcCGAGTGCCATTTCACCAACGGGACG
GAGCGGGTGCGGTtcgTGgaaAGAtACATCTATAACCGGGAGGAGTaCGT
GCGCTTCGACAGCGACGTGGGGGAGTaCCGGGCGGTCACGGAGCTCGGGC
GGCcCGACGCTGAGTCCTGGAACccGCAGAAGGAGCTCTTGGAGCgGgcG
CGGGCCGCGGTGGACACCTACTGCAGAcAcAACTACGGGGTGggcGAGAG
CTTCaCGGTGCAGCGGCGAG
>DRB1*03801
CACATTTCTTGGAGATGgTAAAGTTCGAGTGCCATTTCACCAACGGGACG
GAGCGGGTGCGGCtTCTGgTGAGAGACATCTATAACCGGGAGGAGcACGT
GCGCTTCGACAGCGACGTGGGGGAGTaCCGGGCGGTCACGGAGCTCGGGC
GGCcCGACGCTGAGTaCTGGAACGGGCAGAAGGAGCTCTTGGAGCgGAgG
CGGGCCGaGGTGGACACggtgTGCAGACACAACTACcGGGTGATTGAGAG
cTTCaCGGTGCAGCGGCGAG
>DRB1*04001
CACATTTCTTGAAGATGGTAAAGTTCGAGTGCCATTTCACCAACGGGACG
GAGCGGGTGCGGTTGGTGGAAAGAGACATCTATAACCGGGAGGAGTACGT
GCGCTTCGACAGCGACGTGGGGGAGTTCCGGGCGGTCACGGAGCTCGGGC
GGCCCTCGGCTGAGTACTGGAACGGGCAGAAGGAGATCTTGGAGCAGAGG
CGGGCCGAGGTGGACACGGTGTGCAGACACAACTACGGGGTGATTGAGAG
CTTCGCGGTGCAGCGGCGAG
>DRB1*04101
CACATTTCTTGGAGATGTTAAAGTTCGAGTGCCATTTCACCAACGGGACG
GAGCGGGTGCGGTATCTGATGAGAGACATCTATAACCGGGAGGAGAACGT
GCGCTTCGACAGCGACGTGGGGGAGTTCCGGGCGGTCACGGAGCTCGGGC
GGCGCGACGCTGAGTCCTGGAACGGGCAGAAGGAGCTCTTGGAGCAGAAG
CGGGCCGCGGTGGACACCTACTGCAGACACAACTACGGGGTGATTGAGAG
CTTCGCGGTGCAGCGGCGAG
>DRB1*04201
CACATTTCTTGGAGATGTTAAAGTTCGAGTGCCATTTCACCAACGGGACG
GAGCGGGTGCGGTATCTGGTGAGAGACATCTATAACCGGGAGGAGAACGT
GCGCTTCGACAGCGACGTGGGGGAGTTCCGGGCGGTCACGGAGCTCGGGC
GGCCCGACGCTGAGTACTGGAACGGGCAGAAGGAGCTCTTGGAGCAGAAG
CGGGCCGCGGTGGACACCTACTGCAGACACAACTACGGGGTGATTGAGAG
CTTCGCGGTGCAGCGGCGAG
>DRB1*04301
CACATTTCTTGgAgAtGTTAAAGTTCGAGTGCCaTTTcACCAACGGGACG
GAGCGGGTGCGGTATCTGGTGAGAGACATCTATAACCGGGAGGAGCACGT
GCGCTTCGACAGCGACGTGGGGGAGTACCGGGCGGTCACGGAGCTCGGGC
GGCCCGACGCTGAGTACTGGAACCGGCAGAAGGAGCTCTTGGAGCGGAGG
CGGGCCGAGGTGGACACGGTGTGCAGACACAACTACCGGGTGGGCGAgAG
CTTCACGGTGCAGCGGCGAG
>DRB1*04401
CACATTTCTTGgAGgTGGcAAAGTcCGAGTGCtATTTCACCAACGGGACG
GAGCGGGTGCGGTtagTGgaaAGAtACATCCATAACCGGGAGGAGaaCGT
GCGCTTCGACAGCGACGTGGGGGAGTTCCGGGCGGTCACGGAGCTCGGGC
GGCcCGACGCTGAGTCCTGGAACcGGCAGAAGGAGCTCTTGGAGCAGAgG
CGGGCCGCGGTGGACACCTACTGCAGACACAACTACcGGGTGggcGAGAG
CTTCaCGGTGCAGCGGCGAG
>DRB1*04501
CACATTTCTTGGAGATGTTAAAGTCCGAGTGCTATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCCCGACGCTGAGTCCTGGAACGGGCAGAAGGAGCTCTTGGAGCGGAAG
CGGGCCGAGGTGGACACCTACTGCAGACACAACTACGGGGTGGGCGAGAG
CTTCACGGTGCAGCGGCGAG
>DRB1*04502
CACATTTCTTGGAGATGTTAAAGTCCGAGTGCTATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCCCGACGCTGAGTCCTGGAACGGGCAGAAGGAGCTCTTGGAGCGGAAG
CGGGCCGAGGTGGACACCTACTGCAGACACAACTACGGGGTGATTGAGAG
CTTCACGGTGCAGCGGCGAG
>DRB1*04601
CACATTTCTTGGAGATGTTAAAGTCCGAGTGCTATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGGAG
CGGGCCGCGGTGGACACCTACTGCAGACACAACTACGGGGTGATTGAGAG
CTTCACGGTGCAGCGGCGAG
>DRB1*04701
CACATTTCTTGAAGATGGTAAAGTTCGAGTGCCATTTCACCAACGGGACG
GAGCGGGTGCGGTATCTGATGAGAGACATCTATAACCGGGAGGAGTTCGT
GCGCTTCGACAGCGACGTGGGGGAGTTCCGGGCGGTCACGGAGCTCGGGC
GGCGCGACGCTGAGTCCTGGAACGGGCAGAAGGAGCTCTTGGAGCGGAGG
CGGGCCGCGGTGGACACCTACTGCAGACACAACTACGGGGTGATTGAGAG
CTTCACGGTGCAGCGGCGAG
>DRB1*04801
CACATTTCTTGGAGATGtTAAAGTcCGAGTGCtATTTCACCAACGGGACG
GAGCGGGTGCGGTtcgTGgaaAGAtACATCCATAACCGGGAGGAGAaCgT
GCGCTTCGACAGCGACGTGGGGGAGTaCCGGGCGGTCACGGAGCTCGGGC
GGCCCGACGCTGAGTCCTGGAACCGGCAGAAGGAGCTCTTGGAGCgGAAG
CGGGCCGaGGTGGACACCTACTGCAGACACAACTACgGGGTGattGAGAG
CTTCACGGTGCAGCGGCGAG
>DRB1*04901
CACATTTCTTGAAGATGGTAAAGTTCGAGTGCCATTTCACCAACGGGACG
GAGCGGGTGCGGTTGGTGGAAAGAGACATCTATAACCGGGAGGAGTACGT
GCGCTTCGACAGCGACGTGGGGGAGTTCCGGGCGGTCACGGAGCTCGGGC
GGCCCTCGGCTGAGTACTGGAACGGGCAGAAGGAGCTCTTGGAGCAGAGG
CGGGCCGAGGTGGACACGGTGTGCAGACACAACTACGGGGTGATTGAGAG
CTTCGCGGTGCAGCGGCGAG
>DRB1*05001
CACATTTCTTGGAGATGGTAAAGTCCGAGTGCCATTTCACCAACGGGACG
GAGCGGGTGCGGCTTCTGGTGAGAGACATCTATAACCGGGAGGAGCACGT
GCGCTTCGACAGCGACGTGGGGGAGTACCGGGCGGTCACGGAGCTCGGGC
GGCCCGACGCTGAGTACTGGAACGGGCAGAAGGAGCTCTTGGAGCAGAGG
CGGGCCGAGGTGGACACGGTGTGCAGACACAACTACGGGGTGATTGAGAG
CTTCGCGGTGCAGCGGCGAG
>DRB1*05101
CACATTTCGTGTACCAGTTTAAGGCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACGGGCAGAAGGAGATCTTGGAGCAGGAG
CGGGCAACGGTGGACACCTACTGCAGACACAACTACGGGGTGATTGAGAG
CTTCACGGTGCAGCGGCGAG
>DRB1*05201
CACATTTCTTGGAGATGGTAAAGTTCGAGTGCCATTTCACCAACGGGACG
GAGCGGGTGCGGTATCTGCTGAGAGACATCTATAACCGGGAGGAGATCCT
GCGCTTCGACAGCGACGTGGGGGAGTTCCGGGCGGTCACGGAGCTCGGGC
GGCCCGACGCTGAGTCCTGGAACCGGCAGAAGGAGCTCTTGGAGCAGAAG
CGGGCCGCGGTGGACACCTACTGCAGACACAACTACCGGGTGGGCGAGAG
CTTCACGGTGCAGCGGCGAG
>DRB1*05301
CACATTTCTTGAAGATGGTAAAGTTCGAGTGCCATTTCACCAACGGGACG
GAGCGGGTGCGGTATCTGATGAGAGACATCTATAACCGGGAGGAGTTCGT
GCGCTTCGACAGCGACGTGGGGGAGTACCGGGCGGTCACGGAGCTCGGGC
GGCCCGACGCTGAGTCCTGGAACGGGCAGAAGGAGCTCTTGGAGCAGGAG
CGGGCAACGGTGGACACCTACTGCAGACACAACTACCGGGTGGGCGAGAG
CTTCACGGTGCAGCGGCGAG
>DRB1*05401
CACATTTCTTGGAGGTGGCAAAGTCCGAGTGCTATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCCCGACGCTGAGTCCTGGAACGGGCAGAAGGAGCTCTTGGAGCAGAGG
CGGGCCGCGGTGGACACCTACTGCAGACACAACTACGGGGTGATTGAGAG
CTTCACGGTGCAGCGGCGAG
>DRB1*05501
CACATTTCTTGGAGATGTATAAGGCCGAGTGCCATTTCACCAACGGGACG
GAGCGGGTGCGGTTTCTGGCGAGAAGCATCTATAACCGGGAGGAGTTCGT
GCGCTTCGACAGCGACGTGGGGGAGTACCGGGCGGTCACGGAGCTCGGGC
GGCCCATCGCTGAGTCCTGGAACCGGCAGAAGGAGTTCTTGGAGCGGAGG
CGGGCCGAGGTGGACACCTACTGCAGACACAACTACGGGGTGATTGAGAG
CTTCACGGTGCAGCGGCGAG
>DRB1*05601
CACATTTCTTGGAGGTGGCAAGGCCGAGTGCTATTTCACCAACGGGACGG
AGCGGGTGCGGTTCGTGGAAAAGATACATCCATAACCGGGAGGAGAACGT
GCGCTTCGACAGCGACGTGGGGGAGTACCGGGCGGTCACGGAGCTCGGGC
GGCCCGACGCTGAGTCCTGGAACGGGCAGAAGGAGCTCTTGGAGCGGAAG
CGGGCCGAGGTGGACACCTACTGCAGACACAACTACGGGGTGGGCGAGAG
CTTCACGGTGCAGCGGCGAG
>DRB1*05701
CACATTTCGTGTACCAGTTTAAGCCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTACTGGAACGGGCAGAAGGAGATCTTGGAGCAGGAG
CGGGCAACGGTGGACACCTACTGCAGACACAACTACGGGGTGATTGAGAG
CTTCACGGTGCAGCGGCGAG
>DRB1*05801
CACATTTCGTGTACCAGTTTAAGCCCGAGTGCCATTTCACCAACGGGACG
GAGCGGGTGCGGTTCGTGGAAAGATACATCCATAACCGGGAGGAGATCCT
GCGCTTCGACAGCGACGTGGGGGAGTACCGGGCGGTCACGGAGCTCGGGC
GGCCCGTCGCTGAGTCCTGGAACGGGCAGAAGGAGATCTTGGAGCAGGAG
CGGGCAACGGTGGACACGGTGTGCAGACACAACTACGGGGTGATTGAGAG
>drb1*05901
CACATTTCGTGAAGATGTTTAAGGCCGAGTGCCATTTCACCAACGGGACG
GAGCGGGTGCGGCTTCTGGCGAGAAGCATCTATAACCGGGAGGAGTTCGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGAGG
CGGGCCGAGGTGGACACCTACTGCAGACACAACTACGGGGTGATTGAGAG
CTTCACGGTGCAGCGGCGAG
>drb1*06101
CACATTTCGTGTACCAGTTTAAGCCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTTCCGGGCGGTCACGGAGCTCGGGC
GGCCCTCGGCTGAGTCCTGGAACGGGCAGAAGGAGCTCTTGGAGCAGAAG
CGGGCCGCGGTGGACACCTACTGCAGACACAACTACGGGGTGATTGAGAG
CTTCGCGGTGCAGCGGCGAG
>drb1*06201
CACATTTCTTGGAGATGTTAAAGTTCGAGTGCCATTTCACCAACGGGACG
GAGCGGGTGCGGTATCTGATGAGAGACATCTATAACCGGGAGGAGCACGT
GCGCTTCGACAGCGACGTGGGGGAGTTCCGGGCGGTCACGGAGCTCGGGC
GGCCCGACGCTGAGTACTGGAACCGGCAGAAGGAGCTCTTGGAGCGGAGG
CGGGCCGAGGTGGACACGGTGTGCAGACACAACTACGGGGTGATTGAGAG
CTTCACGGTGCAGCGGCGAG
>DRB1*06301
CACATTTCTTGGAGATGTTAAAGTTCGAGTGCCATTTCACCAACGGGACG
GAGCGGGTGCGGTATCTGATGAGAGACATCTATAACCGGGAGGAGCACGT
GCGCTTCGACAGCGACGTGGGGGAGTTCCGGGCGGTCACGGAGCTCGGGC
GGCCCGACGCTGAGTACTGGAACGGGCAGAAGGAGCTCTTGGAGCGGAAG
CGGGCCGAGGTGGACACGGTGTGCAGACACAACTACGGGGTGATTGAGAG
CTTCACGGTGCAGCGGCGAG
>DRB1*06401
CACATTTCTTGGAGATGTTTAAGTTCGAGTGCTATTTCACCAACGGGACG
GAGCGGGTGCGGCTTCTGGTGAGAAGCATCTATAACCGGGAGGAGTTCGT
GCGCTTCGACAGCGACGTGGGGGAGTACCGGGCGGTCACGGAGCTCGGGC
GGCCCATCGCTGAGTCCTGGAACCGGCAGAAGGAGTTCTTGGAGCAGAGG
CGGGCCGAGGTGGACACGGTGTGCAGACACAACTACGGGGTGGGCGAGAG
CTTCACGGTGCAGCGGCGAG
>DRB1*06501
CACATTTCGTGAGGATGTTTAAGGCCGAGTGCCATTTCACCAACGGGACG
GAGCGGGTGCGGTTTCTGGCGAGAAGCATCTATAACCGGGAGGAGTTCGT
GCGCTTCGACAGCGACGTGGGGGAGTACCGGGCGGTCACGGAGCTCGGGC
GGCCCGACGCTGAGTACTGGAACCGGCAGAAGGAGCTCTTGGAGCAGAAG
CGGGCCGCGGTGGACACCTACTGCAGACACAACTACCGGGTGGGCGAGAG
CTTCACGGTGCAGCGGCGAG
>DRB1*06601
CACATTTCTTGGAGATGTTAAAGTCCGAGTGCCATTTCACCAACGGGACG
GAGCGGGTGCGGCTTCTGGTGAGAGACATCTATAACCGGGAGGAGTTGGT
GCGCTTCGACAGCGACGTGGGGGAGTTCCGGGCGGTCACGGAGCTCGGGC
GGCCCATCGCTGAGTCCTGGAACCGGCAGAAGGAGCTCTTGGAGCGGAGG
CGGGCCGAGGTGGACACGGTGTGCAGACACAACTACGGGGTGATTGAGAG
CTTCACGGTGCAGCGGCGAG
>DRB1*06701
CACATTTCTTGGAGATGTTAAAGtcCGAGTGCCATTTCACCAACGGGACG
GAGCGGGTGCGGTATCTGGTGAGAGACATCTATAACCGGGAGGAGCACGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCGGAGG
CGGGCCGAGGTGGACACCTACTGCAGACACAACTACGGGGTGATTGAGAG
CTTCACGGTGCAGCGGCGAG
>jmadrb-ccah002
CACATTTCTTGGAGATGGTAAAGTTCGAGTGCCATTTCACCAACGGGACG
GAGCGGGTGCGGCTTCTGGTGAGAGACATCTATAACCGGGAGGAGCACGT
GCGCTTCGACAGCGACGTGGGGGAGTACCGGGCGGTCACGGAGCTCGGGC
GGCCCGACGCTAAGTACTACAACGGGCAGAAGGAGCTCTTGGAGCAGAGG
CGGGCCGAGGTGGACACGGTGTGCAAACACAACTACGGGGTGATTGAGAG
CTTCACGGTGCAGCGGCGAG
>jmadrb-d002
CACATTTCTTGGAGATGTTAAAGTCCGAGTGCTATTTCACCAACGGGACG
GAGCGGGTGCGGTTCGTGGAAAGATACATCCATAACCGGGAGGAGCACGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCGGAAG
CGGGCCGCGGTGGACACCTACTGCAGACACAACTACGGGGTGATTGAGAG
CTTCGCGGTGCAGCGGCGAG
>jmadrb-d004
CACATTTCGTGAAGATGTTTAAGGCCGAGTGCCATTTCACCAACGGGACG
GAGCGGGTGCGGCTTCTGGCGAGAAGCATCTATAACCGGGAGGAGTTCGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGAGG
CGGGCCGCGGTGGACACCTACTGCAGACACAACTACGGGGTGATTGAGAG
CTTCACGGTGCAGCGGCGAG
>jmadrb-vg1002
CACATTTCTTGGAGATGTTAAGTCCGAGTGCTATTTCACCAACGGGACGG
AGCGGGTGCGGTTCGTGGAAAAGATACATCCATAACCGGGAGGAGCACGT
GCGCTTCGACAGCGACGTGGGGGAGTACCGGGCGGTCACGGAGCTCGGGC
GGCCCGACGCTGAGTCCTGGAACCAGCAGAAGGAGCTCTTGGAGCGGAAG
CGGGCCGAGGTGGACACCTACTGCAGACACAACTACGGGGTGATTGAGAG
CTTCGCGGTGCAGCGGCGAG
>jsdrb-coy1057a
CACATTTCTTGGAGATGTTAAAGtTCGAGTGCCATTTCACCAACGGGACG
GAGCGGGTGCGGTATCTGATGAGAGACATCTATAACCGGGAGGAGCACGT
GCGCTTCGACAGCGACGTGGGGGAGTTCCGGGCGGTCACGGAGCTCGGGC
GGCCCGACGCTGAGTACTGGAACGGGCAGAAGGAGCTCTTGGAGCGGAGG
CGGGCCGAGGTGGACACGGTGTGCAGACACAACTACGGGGTGATTGAGAG
CTTCACGGTGCAGCGGCGAG
>jsdrb-efin8der
CACATTTCGTGTACCTGTTTAAGCCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACGGGCAGAAGGAGATCTTGGAGCAGGAG
CGGGCAACGGTGGACACCTACTGCAGACACAACTACGGGGTGATTGAGAG
CTTCACGGTGCAGCGGCGAG
>jsdrb-hlat17der
CACATTTCTTGAAGATGGTAAAGTTCGAGTGCCATTTCACCAACGGGACG
GAGCGGGTGCGGTTGGTGGAAAGAGACATCTATAACCGGGAGGAGTACGT
GCGCTTCGACAGCGACGTGGGGGAGTTCCGGGCGGTCACGGAGCTCGGGC
GGCCCTCGGCTGAGTCCTGGAACCGGCAGAAGGAGTTCTTGGAGCAGAGG
CGGGCCGAGGTGGACACGGTgTGCAGACACAACTACCGGGTGGGCGAGAG
CTTCACGGTGCAGCGGCGAG
>jsdrb-oest4der
CACATTTCTTGGAGATGTTAAAGTCCGAGTGCCATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCGGAAG
CGGGCCGAGGTGGACACCTACTGCAGACACAACTACGGGGTGATTGAGAG
CTTCACGGTGCAGCGGCGAG
>jsdrb-ploo1der
CACATTTCTTGaAGATGGTAAAGTTCGAGTGCCATTTCACCAACGGGACG
GAGCGGGTGCGGTTGGTGGAAAGAGACATCTATAACCGGGAGGAGTACGT
GCGCTTCGACAGCGACGTGGGGGAGTTCCGGGCGGTCACGGAGCTCGGGC
GGCCCTCGGCTGAGTACTGGAACGGGCAGAAGGAGCTCTTGGAGCAGAGG
CGGGCCGAGGTGGACACGGTGTGCAGACACAACTACGGGGTGGGCGAGAG
CTTCACGGTGCAGCGGCGAG
>jsdrb-qfinl1der
CACATTTCGTGTACCTGTTTAAGCCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGTCGCTGAGTCCTGGAACGGGCAGAAGGAGATCTTGGAGCAGAAG
CGGGCCGCGGTGGACACCTACTGCAGACACAACTACGGGGTGATTGAGAG
CTTCACGGTGCAGCGGCGAG
>jsdrb-rest6der
CACATTTCTTGGAGGTGGCAAAGTCCGAGTGCTATTTCACCAACGGGACG
GAGCGGGTGCGGTTCGTGGAAAGATACATCCATAACCGGGAGGAGAACGT
GCGCTTCGACAGCGACGTGGGGGAGTACCGGGCGGTCACGGAGCTCGGGC
GGCGCGACGCTGAGTCCTGGAACCGGCAGAAGGAGCTCTTGGAGCGGAAG
CGGGCCGAGGTGGACACCTACTGCAGACACAACTACGGGGTGATTGAGAG
CTTCACGGTGCAGCGGCGAG
>lk03102
CACATTTCTTGAAGATGGTAAAGTTCGAGTGCCATTTCACCAACGGGACG
GAGCGGGTGCGGTATCTGATGAGAGACATCTATAACCGGGAGGAGTTCGT
GCGCTTCGACAGCGACGTGGGGGAGTTCCGGGCGGTCACGGAGCTCGGGC
GGCGCGACGCTGAGTCCTGGAACGGGCAGAAGGAGCTCTTGGAGCAGAAG
CGGGCCGCGGTGGACACCTACTGCAGACACAACTACGGGGTGATTGAGAG
CTTCaCGGTGCAGCGGCGAG
>lk035v-mw-u
CACATTTCGTGAAGATGTTTAAGGCCGAGTGCCATTTCACCAACGGGACG
GAGCGGGTGCGGCTTCTGGCGAGAAGCATCTATAACCGGGAGGAGTTCGT
GCGCTTCGACAGCGACGTGGGGGAGTTCCGGGCGGTCACGGAGCTCGGGC
GGCCCGACGCTGAGTCCTGGAACCGGCAGAAGGAGTTCTTGGAGCAGAGG
CGGGCCGCGGTGGACACCTACTGCAGACACAACTACCGGGTGGGCGAGAG
CTTCACGGTGCAGCGGCGAG
>lkdrb-383-6
CACATTTCGTGGAGGTGTTTAAGGCCGAGTGCCATTTCACCAACGGGACG
GAGCGGGTGCGGTTCGTGGAAAGAAGCATCTATAACCGGGAGGAGTACGT
GCGCTTCGACAGCGACGTGGGGGAGTACCGGGCGGTCACGGAGCTCGGGC
GGCCCGACGCTGAGTACTGGAACCCGCAGAAGGAGCTCTTGGAGCGGGGG
CGGGCCGAGGTGGACACCTACTGCAGACACAACTACGGGGTGGGCGAGAG
CTTCACGGTGCAGCGGCGAG
>lkdrb-383-8
CACATTTCTTGGAGATGTTAAAGTTCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGAGG
CGGGCCGAGGTGGACACCTACTGCAGACACAACTACGGGGTGGGCGAGAG
CTTCACGGTGCAGCGGCGAG
>lkdrb-384-34
CACATTTCTTGAAGATGGTAAAGTTCGAGTGCCATTTCACCAACGGGACG
GAGCGGGTGCGGTTTGTGGAAAGATACATCTATAACCGGGAGGAGTACGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGAGG
CGGGCCGCGGTGGACACCTACTGCAGACACAACTACGGGGTGGGCGAGAG
CTTCACGGTGCAGCGGCGAG
>lkdrb-awd01
CACATTTCTTGAACGTGGCAAAGTCCGAGTGCTATTTCACCAACGGGACG
GAGCGGGTGCGGTTCGTGGACAGATACATCTATAACCGGGAGGAGTTCGT
GCGCTTCGACAGCGACGTGGGGGAGTTCCGGGCGGTCACGGAGCTCGGGC
GGCCCGACGCTGAGTACCTGAACCGGCAGAAGGAGATCTTGGAGCAGGAG
CGGGCCGCGGTGGACACCTACTGCAGACACAACTACGGGGTGGGCGAGAg
CTTCACGGTGCAgCGGCGAg
>lkdrb-awd02
CACATTTCTTGAACGTGGCAAAGTCCGAGTGCTATTTCACCAACGGGACG
GAGCGGGTGCGGTTCGTGGACAGATACATCTATAACCGGGAGGAGTTCGT
GCGCTTCGACAGCGACGTGGGGGAGTTCCGGGCGGTCACGGAGCTCGGGC
GGCCCGACGCTGAGTACctGAACCGGCAGAAGGAGATCTTGGAGCAGGAG
CGGGCCGCGGTGGACACcTACTGCAGACACAACTACGGGGTGattGAGAg
CTTCACGGTGCAgCGGCGAg
>lkdrb-awd03
CACATTTCgTGtACcaGtttAAGggCGAGTGCTATTTCACCAACGGGACG
GAGCGGGTGCGGcTtCTGGcgAGAagCATCTATAACCGGGAGGAGTTCGT
GCGCTTCGACAGCGACGTGGGGGAGTTCCGGGCGGTCACGGAGCTCGGGC
GGCCCGACGCTGAGTACtgGAACCGGCAGAAGGAGcTCTTGGAGCAGagG
CGGGCCGCGGTGGACACcTAcTGCAGACACAACTACGGGGTGattGAGAg
CTTCACGGTGCAgCGGCGAg
>lkdrb-awd04
CACATTTCTTGAACGTGGCAAAGTCCGAGTGCTATTTCACCAACGGGACG
GAGCGGGTGCGGTTCGTGGACAGATACATCTATAACCGGGAGGAGTTCGT
GCGCTTCGACAGCGACGTGGGGGAGTTCCGGGCGGTCACGGAGCTCGGGC
GGCCCGACGCTGAGTACTGGAACCGGCAGAAGGAGATCTTGGAGCAGGAG
CGGGCCGCGGTGGACACCTACTGCAGACACAACTACGGGGTGATTGAGAg
CTTCACGGTGCAgCGGCGAg
>lkdrb-coy-r
CACATTTCTTGGAGGTGGCAAAGtyCGAGTGCCATTTCACCAACGGGACG
GAGCGGGTGCGGTTCGTGGAAAGATACATCcATAACCGGGAGGAGTtCGT
GCGCTTCGACAGCGACGTGGGGGAGTACCGGGCGGTCACGGAGCTCGGGC
GGCCCGACGCTGAGTCCTGGAACgGGCAGAAGGAGcTCTTGGAGCAGGAG
CGGGCcgCGGTGGACACctacTGCAGACACAACTACCGGGTGGGCGAGAG
CTTCACGGTGCAGCGGCGAG
>lkdrb-coy-v
CACATTTCTTGGAGATGTtAAAGTtCGAGTGCcATTTCACCAACGGGACG
GAGCGGGTGCGGTatcTGGtgAGAgACATCtATAACCGGGAGGAGcACGT
GCGCTTCGACAGCGACGTGGGGGAGTtCCGGGCGGTCACGGAGCTCGGGC
GGCCCGACGCTGAGTaCTGGAACGGGCAGAAGGAGCTCTTGGAGCAGAAG
CGGGCCGcGGTGGACACCTACTGCAGACACAACTACGGGGTGattGAGAG
CTTCgCGGTGCAGCGGCGAG
>lkdrb-coy-x
CACATTTCTTGGAGGTGGCAAAGgyCGAGTGCCATTTCACCAACGGGACG
GAGCGGGTGCGGTTCGTGGAAAGATACATCtATAACCGGGAGGAGTaCGT
GCGCTTCGACAGCGACGTGGGGGAGTACCGGGCGGTCACGGAGCTCGGGC
GGCCCGACGCTGAGTCCTGGAACcGGCAGAAGGAGaTCTTGGAGCAGGAG
CGGGCaaCGGTGGACACggtgTGCAGACACAACTACgGGGTGGGCGAGAG
CTTCACGGTGCAGCGGCGAG
>lkdrb-015v-c13
CACATTTCTTGAAGATGGTAAAGTTCGAGTGCCATTTCACCAACGGGACG
GAGCGGGTGCGGCTTCTGGTGAGAGACATCTATAACCGGGAGGAGCACGT
GCGCTTCGACAGCGACGTGGGGGAGTACCGGGCGGTCACGGAGCTCGGGC
GGCCCGACGCTGAGTACTGGAACGGGCAGAAGGAGCTCTTGGAGCAGAGG
CGGGCCGAGGTGGACACGGTGTGCAGACACAACTACGGGGTGATTGAGAG
CTTCACGGTGCAGCGGCGAG
>lkdrb-01802
CACATTTCTTGGAGGTGGCAAAGTCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGGCGCTGAGTCCTGGAACCGGCAGAAGGAGCTCTTGGAGCGGAAG
CGGGCCGAGGTGGACACGGTGTGCAGACACAACTACGGGGTGATTGAGAG
CTTCACGGTGCAGCGGCGAG
>lkdrb-048v
CACATTTCTTGGAGATGtTAAAGTcCGAGTGCtATTTCACCAACGGGACG
GAGCGGGTGCGGTtcgTGgaaAGAtACATCcATAACCGGGAGGAGcaCgT
GCGCTTCGACAGCGACGTGGGGGAGTaCCGGGCGGTCACGGAGCTCGGGC
GGCCCGACGCTGAGTCCTGGAACCGGCAGAAGGAGCTCTTGGAGCgGAAG
CGGGCCGaGGTGGACACCTACTGCAGACACAACTACgGGGTGattGAGAG
CTTCgCGGTGCAGCGGCGAG
>lkdrb-2332
CACATTTCTTGGAGaTGGtAAAGttCGAGTGCCATTTCACCAACGGGACG
GAGCGGGTGCGGTatcTGGAAAGATACATCTATAACCGGGAGGAGatCGT
GCGCTTCGACAGCGACGTGGGGGAGTACCGGGCGGTCACGGAGCTCGGGC
GGCCCatCGCTGAGTcCTGGAACCgGCAGAAGGAGCTCTTGGAGCaGagG
CGGGCCGcGGTGGACACCTACTGCAGACACAACTACGGGGTGattGAGAG
CTTCACGGTGCAGCGGCGAG
>lkdrb-5078
CACATTTCTTGGAgATGTTAAAGTtcgAgTGCCATtTCAcCAAcggGacg
gaGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACCGGCAGAAGGAGCTCTTGGAGCAGGAG
CGGGCCGCGGTGGACACGGTGTGCAGACACAACTACCGGGTGGGCGAGAG
CTTCACGGTGCAGCGGCGAG
>lkdrb-9050
CACATTTCTTGGAGaTGGtAAAGTtCGAGTGCCATTTCACCAACGGGACG
GAGCGGGTGCGGCTtcTGGtgAGAgACATCtATAACCGGGAGGAGCaCGT
GCGCTTCGACAGCGACGTGGGGGAGTtCCGGGCGGTCACGGAGCTCGGGC
GGCCCGaCGCTGAGTaCTGGAACGGGCAGAAGGAGATCTTGGAGCAGGAG
CGGGCAACGGTGGACACCTACTGCAGACACAACTACGGGGTGATTGAGAG
CTTCACGGTGCAGCGGCGAG
>lkdrb-a79
CACATTTCGTGAAGATGTTTAAGGCCGAGTGCCATTTcAccAAcGGGAcG
GAGcGGGTGcGGcTTcTGGcGAGAgacATCTATAACCGGGAGGAGTTCGT
GCGCTTCGACAGCGACGTGGGGGAGTACCGGGCGGTCACGGAGCTCGGGC
GGCGCGACGCTGAGTCCTGGAACCGGCAGAAGGAGCTCTTGGAGOGGGCG
CGGGCCGCGGTGGACACCTACTGCAGACACAACTACCGGGTGGGCGAGAG
CTTCACGGTGCAGCGGCGAG
>lkdrb-D7v
CACATTTCTTGGAGATGGTAAAGTTCGAGTGCCATTTCACCAACGGGACG
GAGCGGGTGCGGTATgTGCTGAGAGACATCTATAACCGGGAGGAGATCgT
GCGCTTCGACAGCGACGTGGGGGAGTTCCGGGCGGTCACGGAGCTCGGGC
GGCCCGACGCTGAGTCCTGGAACCGGCAGAAGGAGCTCTTGGAGCAGAAG
CGGGCCGCGGTGGACACCTACTGCAGACACAACTACCGGGTGGGCGAGAG
CTTCACGGTGCAGCGGCGAG
>lkdrb-E17
CACATTTCgTGtAccaGttgAAGcCCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGTCGCTGAGTCCTGGAACGGGCAGAAGGAGCTCTTGGAGCAGGAG
CGGGCCgCGGTGGACACCTACTGCAGACACAACTACGGGGTGATTGAGAG
CTTCACGGTGCAGCGGCGAG
>lkdrb-E25
CACATTTCgTGaAGaTGGCtAAGgCCGAGTGCcATTTCACCAACGGGACG
GAGCGGGTGCGGTTtcTGGcAAGAaACATCtATAACCGGGAGGAGtTCGT
GCGCTTCGACAGCGACGTGGGGGAGTtCCGGGCGGTCACGGAGCTCGGGC
GGCCCGaCGCTGAGTCCTGGAACCGGCAGAAGGAGCTCTTGGAGCgGGAG
CGGGCCgCGGTGGACACCTACTGCAGACACAACTACCGGGTGggCGAGAG
CTTCACGGTGCAGCGGCGAG
>lkdrb-E7
CACATTTCTTGaAGaTGGtAAAGTCCGAGTGCTATTTCACCAACGGGACG
GAGCGGGTGCGGTTCGTGGAAAGATACATCCATAACCGGGAGGAGCTCGT
GCGCTTCGACAGCGACGTGGGGGAGTtCCGGGCGGTCACGGAGCTCGGGC
GGCCCGaCGCTGAGTCCTGGAACcGGCAGAAGGAGcTCTTGGAGCgGaAG
CGGGCcgaGGTGGACACggtgTGCAGACACAACTACGGGGTGATTGAGAG
CTTCACGGTGCAGCGGCGAG
>lkdrb-E25-2nd
CACATTTCgTGaAGaTGtttAAGtCCGAGTGCcATTTCACCAACGGGACG
GAGCGGGTGCGGTatcTGGcgAGAgACATCtATAACCGGGAGGAGtTCGT
GCGCTTCGACAGCGACGTGGGGGAGTtCCGGGCGGTCACGGAGCTCGGGC
GGCCCGaCGCTGAGTCCTGGAACcGGCAGAAGGAGcTCTTGGAGCgGGcG
CGGGCcgCGGTGGACACCTACTGCAGACACAACTACcGGGTGggcGAGAG
CTTCACGGTGcAGcGGcGAG
>lkdrb-gw-c
CACATTTCTTGGAGATGTTAAAGGCCGAGTGCCATTTCACCAACGGGACG
GAGCGGGTGCGGTATCTGGTGAGAGACATCTATAACCGGGAGGAGTTGGT
GCGCTTCGACAGCGACGTGGGGGAGTTCCGGGCGGTCACGGAGCTCGGGC
GGCCCGACGCTGAGTACTGGAACGGGCAGAAGGAGATCTTGGAGCGGAGG
CGGGCCGAGCTGGACACGGTGTGCAGACACAACTACGGGGTGATTGAGAG
CTTCACGGTGCAGCGGCGAG
>lkdrb-gw-n
CACATTTCTTGGAGATGTTAAAGTTCGAGTGCCATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCGACGCTGAGTCCTGGAACCGGCAGAAGGAGTTCTTGGAGCAGAGG
CGGGCCGAGGTGGACACCTACTGCAGACACAACTACGGGGTGGGCGAGAG
CTTCACGGTGCAGCGGCGAG
>lkdrb-307
CACATTTCTTGaAGATGtcAAAGTCCGAGTGCtATTTCACCAACGGGACG
GAGCGGGTGCGGttggTGGaaAGAtgCATCTATAACCGGGAGGAGtaCGT
GCGCTTCGACAGCGACGTGGGGGAGTACCGGGCGGTCACGGAGCTCGGGC
GGCCCtcgGCTGAGTcCTGGAACGGGCAGAAGGAGtTCTTGGAGCAGAaG
CGGGCCGaGGTGGACACggtgTGCAGACACAACTACGGGGTGggcGAGAG
CTTCaCGGTGCAGCGGCGAG
>lkdrb-048v2
CACATTTCTTGGAGATGtTAAAGTcCGAGTGCtATTTCACCAACGGGACG
GAGCGGGTGCGGTtcgTGgaaAGAtACATCcATAACCGGGAGGAGcaCgT
GCGCTTCGACAGCGACGTGGGGGAGTaCCGGGCGGTCACGGAGCTCGGGC
GGCCCGACGCTGAGTCCTGGAACCGGCAGAAGGAGCTCTTGGAGCgGAAG
CGGGCCGaGGTGGACACCTACTGCAGACACAACTACgGGGTGattGAGAG
CTTCACGGTGCAGCGGCGAG
>lkdrb-7573
CACATTTCTTGGAGGTGGCAAAGTCCGAGTGCTATTTCACCAACGGGACG
GAGCGGGTGCGGTTCGTGGAAAGATACATCtATAACCGGGAGGAGTaCGT
GCGCTTCGACAGCGACGTGGGGGAGTACCGGGCGGTCACGGAGCTCGGGC
GGCgCGaCGCTGAGTCCTGGAACcGGCAGAAGGAGCTCTTGGAGCgGaAG
CGGGCcgCGGTGGACACCTACTGCAGACACAACTACcGGGTGggcGAGAG
CTTCACGGTGCAGCGGCGAG
>lkdrb-7669
CACATTTCTTGGAGaTGGtAAAGTCCGAGTGCTATTTCACCAACGGGACG
GAGCGGGTGCGGTTCGTGGAAAGATACATCCATAACCGGGAGGAGctCGT
GCGCTTCGACAGCGACGTGGGGGAGTaCCGGGCGGTCACGGAGCTCGGGC
GGCCCGACGCTGAGTCCTGGAACCGGCAGAAGGAGcTCTTGGAGCGGAAG
CGGGCCGaGGTGGACACggtgTGCAGACACAACTACGGGGTGATTGAGAG
CTTCACGGTGCAGCGGCGAG
>lkdrb-3166
CACATTTCGTGAGGATGTATAAGGCCGAGTGCCATTTCACCAACGGGACG
GAGCGGGTGCGGTaTCTGatGAGAgaCATCTATAACCGGGAGGAGTTCGC
GCGCTTCGACAGCGACGTGGGGGAGTACCGGGCGGTCACGGAGCTCGGGC
GGCGCGACGCTGAGTCCTGGAACCGGCAGAAGGAGCTCTTGGAGCGGAGG
CGGGCCGAGGTGGACACCTACTGCAGACACAACTACGGGGTGATTGAGAG
CTTCACGGTGCAGCGGCGAG
>lkdrb3180
CACATTTCTTGGAGGTGGCAAAGTCCGAGTGCtATTTCACCAACGGGACG
GAGCGGGTGCGGTTCGTGGAAAGATACATCCATAACCGGGAGGAGTTCGT
GCGCTTCGACAGCGACGTGGGGGAGTACCGGGCGGTCACGGAGCTCGGGC
GGCCCtcgGCTGAGTCCTGGAACgGGCAGAAGGAGaTCTTGGAGCaGgAG
CGGGCaacGGTGGACACCTACTGCAGACACAACTACGGGGTGATTGAGAG
CTTCACGGTGCAGCGGCGAG
>lkdrbper475
CACATTTCTTGaAGATGGTAAAGTTCGAGTGCCATTTCACCAACGGGACG
GAGCGGGTGCGGtTggTGGaaAGAGACATCTATAACCGGGAGGAGtACGT
GCGCTTCGACAGCGACGTGGGGGAGTACCGGGCGGTCACGGAGCTCGGGC
GGCCCtcgGCTGAGTcCTGGAACcGGCAGAAGGAGtTCTTGGAGCAGAGG
CGGGCCGcGGTGGACACctacTGCAGACACAACTACGGGGTGggCGAGAG
CTTCaCGGTGCAGCGGCGAG
>drb-lk-ew31
CACATTTCGTGTACCAGTTTAAGGGCGAGTGCCATTTCACCAACGGGACG
GAGCGGGTGCGGTTTCTGGCGAGAAGCATCTATAACCGGGAGGAGTTCGT
GCGCTTCGACAGCGACGTGGGGGAGTACCGGGCGGTCACGGAGCTCGGGC
GGCCCATCGCTGAGTCCTGGAACCGGCAGAAGGAGTTCTTGGAGCGGAGG
CGGGCCGAGGTGGACACCTACTGCAGACACAACTACGGGGTGATTGAGAG
cTTCACGGTGCAGcggcgag
>drb-lk-ew56b
CACATTTCtTGgAggtGgcaAAGtcCGAGTGCtATTTCACCAACGGGACG
GAGCGGGTGCGGTTcgTGGaaAGAtaCATCcATAACCGGGAGGAGaaCGT
GCGCTTCGACAGCGACGTGGGGGAGTtCCGGGCGGTCACGGAGCTCGGGC
GGCCCgaCGCTGAGTaCTGGAACgGGCAGAAGGAGcTCTTGGAGCaGAaG
CGGGCCGcGGTGGACACCTACTGCAGACACAACTACGGGGTGggcGAGAG
cTTCACGGTGCAGcggcgag
>drb-lk-ew73b
CACATTTCGTGaggatGTTTAAGGCCGAGTGCtATTTCACCAACGGGACG
GAGCGGGTGCGGTTggTGGaaAGAgaCATCTATAACCGGGAGGAGTTCGT
GCGCTTCGACAGCGACGTGGGGGAGTACCGGGCGGTCACGGAGCTCGGGC
GGCCCgaCGCTGAGTaCTGGAACgGGCAGAAGGAGcTCTTGGAGCaGAGG
CGGGCCGAGGTGGACACCTACTGCAGACACAACTACcGGGTGggcGAGAG
cTTCACGGTGcAGcggcgag
>drb-lk-ew88b
CACATTTCgTGaggatGTTTAAGGcCGAGTGCtATTTCACCAACGGGACG
GAGCGGGTGCGGTTggTGGaaAGAgaCATCTATAACCGGGAGGAGTaCGT
GCGCTTCGACAGCGACGTGGGGGAGTACCGGGCGGTCACGGAGCTCGGGC
GGCCCATCGCTGAGTCCTGGAACCGGCAGAAGGAGTTCTTGGAGCaGAGG
CGGGCCGcGGTGGACACCTACTGCAGACACAACTACcGGGTGggCGAGAG
cTTCACGGTGCAGcggcgag
>drb-lk-8187
CACATTTCTTGGAGATGGTAAAGTTCGAGTGCCATTTCACCAACGGGACG
GAGCGGGTGCGGTATCTGGCGAGAGACATCTATAACCGGGAGGAGATCCT
GCGCTTCGACAGCGACGTGGGGGAGTTCCGGGCGGTCACGGAGCTCGGGC
GGCCCATCGCTGAGTCCTGGAACCGGCAGAAGGAGATCTTGGAGCAGAGG
CGGGCCGCGGTGGACACCTACTGCAGACACAACTACGGGGTGATTGAGAG
CTTCGCGGTGCAGcGGCgAg

1. A method for diagnosing susceptibility to diabetes in a non-human animal, the method comprising:
(a) identifying whether or not a polymorphism as defined in Table 4 or a polymorphism which is in linkage disequilibrium with such a polymorphism is present in the genome of the animal; and
(b) thereby diagnosing whether the animal is susceptible to diabetes, wherein optionally the said identifying is carried out on a sample from the animal.

2. A method according to claim 1, comprising identifying whether all of the polymorphisms in any given row of Table 4 are present in the animal.

3. A method according to claim 1, wherein if the animal is identified as having the polymorphism it is further tested to determine whether it has aberrant levels of glucose in its blood.

4. A method according to any one of the preceding claims, wherein the animal is a dog, optionally of any of the following breeds: Samoyed, Tibetan Terrier, Bichon Frise, Yorkshire Terrier, Schnauzer (miniature), Border Collie, Dachshund, Border Terrier or Poodle; or a dog that is genetically related to any of these breeds.

5. A method according to claim 1, wherein step (a) comprises contacting a polynucleotide of the animal with a specific binding agent for the polymorphism and determining whether the agent binds to the polynucleotide, wherein binding of the agent to the polynucleotide indicates the presence of the polymorphism.

6. A method according to claim 5 wherein the agent is a polynucleotide which is able to bind a polynucleotide comprising the polymorphism but which does not bind a polynucleotide that does not comprise the polymorphism.

7. A method according to claim 1, wherein step (a) comprises contacting a polypeptide of the animal with a specific binding agent for a polypeptide that comprises a sequence encoded by a polymorphism as defined in Table 4.

8. A method according to claim 1, wherein the polymorphism is detected by measuring the mobility of a polynucleotide of the animal or of a polypeptide of the animal which is encoded by a polynucleotide comprising the polymorphism.

9. A probe or primer which is capable of detecting a polymorphism as defined in claim 1, or an antibody which is capable of detecting (and is specific for) a polypeptide encoded by the polymorphism.

10. (canceled)

11. A kit for carrying out the method of claim 1 comprising a probe, primer or antibody according to claim 9.

12. A method of preparing customised food for an animal which is susceptible to diabetes, the method comprising:
(a) determining whether the animal is susceptible to diabetes by a method according to claim 1; and
(b) preparing food suitable for the animal, and optionally wherein the customized animal food comprises ingredients which prevent or alleviate diabetes, and/or does not comprise ingredients which contribute to or aggravate diabetes.

13. (canceled)

14. A method according to claim 12 wherein the customised animal food comprises a low level of simple carbohydrate, wherein the carbohydrate is optionally a monosaccharide or a polysaccharide.

15. A method according to claim 12, further comprising providing the food to the animal, the animal's owner or the person responsible for feeding the animal.

16. A method of providing a customized animal food, comprising providing food suitable for an animal which is susceptible to diabetes to the animal, the animal's owner or the person responsible for feeding the animal, wherein the animal has been genetically determined to be susceptible to diabetes, optionally by the method of claim 1.

17. A method for identifying an agent for the treatment of diabetes, the method comprising:
(a) contacting a polypeptide encoded by a polynucleotide comprising a polymorphism as defined in claim 1 with a test agent; and
(b) determining whether the agent is capable of binding to the polypeptide or modulating the activity or expression of the polypeptide.

18. (canceled)

19. A method of treating an animal for diabetes, the method comprising administering to the animal an effective amount of a therapeutic compound which prevents or treats the disorder, wherein the animal has a polymorphism as defined in any one of claims 1 and 4, and optionally has been identified as being susceptible to diabetes by a method according to claim 1 and optionally the compound is insulin.

20. A database comprising information relating to one or more polymorphisms as defined in claim 1 and optionally also their association with diabetes.

21. A method for determining whether an animal is susceptible to diabetes, the method comprising:
(a) inputting data of one or more polymorphisms of the animal to a computer system;
(b) comparing the data to a computer database, which database comprises information relating to the polymorphisms defined in claim 1; and
(c) determining on the basis of the comparison whether the animal is susceptible to diabetes.

22. A computer program encoded on a computer-readable medium and comprising program code which, when executed, performs all the steps of claim 21, or a computer system arranged to perform a method according to claim 21 comprising:
(a) means for receiving data of the one or more polymorphisms present in the animal;
(b) a module for comparing the data with a database comprising information relating to one or more polymorphism as defined in claim 1; and
(c) means for determining on the basis of said comparison whether is susceptible to diabetes.

23-25. (canceled)

26. A method of preparing customised food for an animal which is susceptible to diabetes, the method comprising:
(a) determining whether the animal is susceptible to diabetes by a method according to claim 1 or 21 and;
(b) electronically generating a customised animal food formulation suitable for the animal;
(c) generating electronic manufacturing instructions to control the operation of food manufacturing apparatus in accordance with the customized animal food formulation; and
(d) manufacturing the customized animal food according to the electronic manufacturing instructions.

27. A computer system according to claim 22, further comprising:
(d) means for electronically generating a customized animal food formulation suitable for the animal;
(e) means for generating electronic manufacturing instructions to control the operation of food manufacturing apparatus in accordance with the customized animal food formulation; and
(f) a food product manufacturing apparatus.

28. Use of a computer system as defined in claim 27 to make a customized animal food product.


SEQUENCE LISTING (243 sequences)

SEQ ID NO: 1 - DNA, 246 bp, Canis familiaris
gaccatgttgccaactacggcataaatgtctaccagtcttacggtccctctggccagtacacccatgaatttgatggcgatgaggagttctacgtggacctggagaagaaggaaactgtctggcggctgcctgtgtttagcacatttagaagttttgacccacagggtgcactgagaaacttggctataataaaacaaaacttgaacatcatgactaaaaggtccaaccaaactgctgctaccaat

SEQ ID NO: 2 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggctataacaaaacaaaacttgaacatcatgactaaaaggtccaacaaaactgctgctaccaat

SEQ ID NO: 3 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggccagagcaaaacaaaacttgaacatcctgactaaaagttccaaccaaactgctgctaccaat

SEQ ID NO: 4 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggctataataaaacaaaacttgaacatcctgactaaaaggtccaaccaaactgctgctaccaat

SEQ ID NO: 5 - DNA, 246 bp, Canis familiaris
gaccatgttgcctactacggcataaatgtctaccagtcttacggtccctctggccagttcacccatgaatttgatggcgatgaggagttctacgtggacctggagaagaaggaaactgtctggcggctgcctgtgtttagcacatttacaagttttgacccacagggtgcactgagaaacttggctataacaaaacaaaacttgaacatcatgactaaaaggtccaacaaaactgctgctaccaat

SEQ ID NO: 6 - DNA, 246 bp, Canis familiaris
gaccatgttgcctactacggcataaatgtctaccagtcttacggtccctctggccagttcacccatgaatttgatggcgatgaggagttctacgtggacctggagaagaaggaaactgtctggcggctgcctgtgtttagcacatttacaagttttgacccacagggtgcgctgagaaacttggctataacaaaacaaaacttgaacatcatgactaaaaggtccaacaaaactgctgctaccaat

SEQ ID NO: 7 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttagaagttttgacccacagggtgcactgagaaacttggctataataaaacaaaacttgaacatcctgactaaaaggtccaaccaaactgctgctaccaat

SEQ ID NO: 8 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggctataacaaaacaaaacttgaacatcatgactaaaaggtccaaccaaactgctgctaccaat

SEQ ID NO: 9 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggccagagcaaaacaaaacttgaacatcctgactaaaaggtccaaccaaactgctgctaccaat

SEQ ID NO: 10 - DNA, 246 bp, Canis familiaris
gaccatgttgcctactacggcataaatgtctaccagtcttacggtccctctggccagttcacccatgaatttgatggcgatgaggagttctacgtggacctggagaagaaggaaactgtctggcggctgcctgtgtttagcacatttagaagttttgacccacagggtgcactgagaaacttggctataataaaacaaaacttgaacatcatgactaaaaggtccaaccaaactgctgctaccaat

SEQ ID NO: 11 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttagaagttttgacccacagggtgcactgagaaacttggctatagcaaaacaaaacttgaacatcctgactaaaagttccaaccaaactgctgctaccaat

SEQ ID NO: 12 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggctataataaaacaaaacttgaacatcatgactaaaaggtccaacaaaactgctgctaccaat

SEQ ID NO: 13 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttgcaagttttgacccacagggtgcactgagaaacttggctatagcaaaacaaaacttgaacatcatgactaaaaggtccaaccaaactgctgctaccaat

SEQ ID NO: 14 - DNA, 246 bp, Canis familiaris
gaccatgttgcctactacggcataaatgtctaccagtcttacggtccctctggccagtacacccatgaatttgatggcgatgaggaattctacgtggacctggagaagaaggaaactgtctggcggctgcctgtgtttagcacatttgcaagttttgacccacagggtgcactgagaaacttggctatagcaaaacaaaacttgaacatcatgactaaaaggtccaaccaaactgctgctaccaat

SEQ ID NO: 15 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttagaagttttgacccacagggtgcactgagaaacttggctataacaaaacaaaacttgaacatcatgactaaaaggtccaacaaaactgctgctaccaat

SEQ ID NO: 16 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttagaagttttgacccacagggtgcactgagaaacttggctataataaaacaaaacttgaacatcatgactaaaaggtccaaccaaactgctgctaccaat

SEQ ID NO: 17 - DNA, 246 bp, Canis familiaris
gaccatgttgcctactacggcataaatgtctaccagtcttacggtccctctggccagtacacacatgaatttgatggcgatgaggagttctacgtggacctggagaagaaggaaactgtctggcggctgcctgtgtttagcacatttagaagttttgacccacagggtgcactgagaaacttggctataataaaacaaaacttgaacatcatgactaaaaggtccaaccaaactgctgctaccaat

SEQ ID NO: 18 - DNA, 246 bp, Canis familiaris
gaccatgttgccaactacggcataaatgtctaccagtcttacggtccctctggccagttcacccatgaatttgatggcgatgaggagttctacgtggacctggagaagaaggaaactgtctggcggctgcctgtgtttagcacatttagaagttttgacccacagggtgcactgagaaacttggctataataaaacaaaacttgaacatcatgactaaaaggtccaaccaaactgctgctaccaat

SEQ ID NO: 19 - DNA, 246 bp, Canis familiaris
gaccatgttgcctactacggcataaatgtctaccagtcttacggtccctctggccagttcacccatgaatttgatggcgatgaggagttctacgtggacctggagaagaaggaaactgtctggcggctgcctgtgtttagcacatttacaagttttgacccacagggtgcactgagaaacttggctataacaaaacaaaacttgaacatcatgactaaaaggtccaaccaaactgctgctaccaat

SEQ ID NO: 20 - DNA, 246 bp, Canis familiaris
gaccatgttgcctactacggcataaatgtctaccagtcttacggtccctctggccagtacacccatgaatttgatggcgatgagttgttctacgtggacctggagaagaaggaaactgtctggcggctgcctgtgtttagcacatttacaagttttgacccacagggtgcactgagaaacttggctataataaaacaaaacttgaacatcctgactaaaaggtccaaccaaactgctgctaccaat

SEQ ID NO: 21 - DNA, 246 bp, Canis familiaris
gaccatgttgccaactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggccataacaaaacaaaacttgaacatcatgactaaaaggtccaacaaaactgctgctaccaat

SEQ ID NO: 22 - DNA, 246 bp, Canis familiaris
gaccatgttgcctactacggcataaatgtctaccagtcttacggtccctctggccagtacacacatgaatttgatggcgatgaggagttctacgtggacctggagaagaaggaaactgtctggcggctgcctgtgtttagcacatttacaagttttgacccacagggtgcactgagaaacttggctataataaaacaaaacttgaacatcatgactaaaaggtccaaccaaactgctgctaccaat

SEQ ID NO: 23 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggctataataaaacaaaacttgaacatcatgactaaaaggtccaaccaaactgctgctaccaat

SEQ ID NO: 24 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggctatagcaaaacaaaacttgaacatcctgactaaaagttccaaccaaactgctgctaccaat

SEQ ID NO: 25 - DNA, 246 bp, Canis familiaris
gaccatgttgccaactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggctataataaaacaaaacttgaacatcatgactaaaaggtccaaccaaactgctgctaccaat

SEQ ID NO: 26 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttgcaagttttgacccacagggtgcactgagaaacttggctagagcaaaacaaaacttgaacatcctgactaaaagttccaaccaaactgctgctaccaat

SEQ ID NO: 27 - DNA, 246 bp, Canis familiaris
gaccatgttgcctactacggcataaatgtctaccagtcttacggtccctctggccagttcacccatgaatttgatggcgatgaggagttctacgtggacctggagaagaaggaaactgtctggcggctgcctgtgtttagcacatttacaagttttgacccacagggtgcactgagaaacttggctataataaaacaaaacttgaacatcatgactaaaaggtccaaccaaactgctgctaccaat

SEQ ID NO: 28 - DNA, 246 bp, Canis familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggctatagcaaaacaaaacttgaacatcatgactaaaaggtccaacaaaactgctgctaccaat

SEQ ID NO: 29 - DNA, 246 bp, Canis familiaris
gaccatgttgccaactacggcataaatgtctaccagtcttacggtccctctggccagttcacccatgaatttgatggcgatgaggagttctatgtggacctggagaagaaggaaactgtctggcggctgcctgtgtttagcacatttagaagttttgacccacagggtgcactgagaaacttggctataataaaacaaaacttgaacatcctgactaaaaggtccaaccaaactgctgctaccaat

SEQ ID NO: 30 - DNA, 246 bp, Canis familiaris
gaccatgttgcctactacggcataaatgtctaccagtcttacggtccctctggccagtacacccatgaatttgatggcgatgagttgttctacgtggacctggagaagaaggaaactgtctggcggctgcctgtgtttagcacatttacaagttttgacccacagggtgcactgagaaacttggctatagcaaaacaaaacttgaacatcctgactaaaaggtccaaccaaactgctgctaccaat

SEQ ID NO: 31 - DNA, 267 bp, Canis familiaris
gatttcgtgtaccagtttaagggcgagtgctatttcaccaacgggacggagcgggtgcggcttctgacgagagacatctataaccgggaggagcacgtgcgcttcgacagcgacgtgggggagtaccgggcggtcacggagctcgggcggccggacgctgagtactggaacgggcagaaggagctcttggagcggaggcgggccgaggtggacacggtgtgcagacacaactacgggagggaagagctcaccacgttgcagcggcga

SEQ ID NO: 32 - DNA, 267 bp, Canis familiaris
gatttcgtgttccagtataaggccgagtgctatttcaccaacgggacggagcgggtgcggcttctgactaaatacatctataaccgggaggagttcgtgcgcttcgacagcgacgtgggggagttccgggcggtcacggagctcgggcggcccgacgctgagtactggaaccgacagaaggacgagatggaccgggtacgggccgagctggacacggtgtgcagacacaactacgggagggaagagctcaccacgttgcagcggcga

SEQ ID NO: 33 - DNA, 267 bp, Canis familiaris
gatttcgtgtaccagtttaaggccgagtgctatttcaccaacgggacggagcgggtgcggcttctgactaaatacatctataaccgggaggagtacgtgcgcttcgacagcgacgtgggggagtaccgggcggtcacggagctcgggcggccctcggctgagtactggaacccgcagaaggacgagatggaccgggtacgggccgagctggacacggtgtgcagacacaactacgggttggaagagctcaccacgttgcagcggcga

SEQ ID NO: 34 - DNA, 267 bp, Canis familiaris
gatttcgtgttccagtttaagggcgagtgctatttcaccaacgggacggagcgggtgcggcttctgactaaatacatctataaccgggaggagtacgtgcgcttcgacagcgacgtgggggagtaccgggcggtcacggagctcgggcggccctgggctgagtactggaacccgcagaaggacgagatggaccgggtacgggccgagctggacacggtgtgcagacacaactacgggttggaagagctctacacgttgcagcggcga

SEQ ID NO: 35 - DNA, 267 bp, Canis familiaris
gatttcgtgttccagtataaggccgagtgctatttcaccaacgggacggagcgggtgcggcttctgactaaatacatctataaccgggaggagtacgtgcgcttcgacagcgacgtgggggagtaccgggcggtcacggagctcgggcggccctgggctgagtactggaacccgcagaaggacgagatggaccgggtacgggccgagctggacacggtgtgcagacacaactacgggttggaagagctcaccacgttgcagcggcga

SEQ ID NO: 36 - DNA, 267 bp, Canis familiaris
gatttcgtgttccagtataaggccgagtgctatttcaccaacgggacggagcgggtgcggcttctgactaaatacatctataaccgggaggagtacgtgcgcttcgacagcgacgtgggggagttccgggcggtcacggagctcgggcggccctgggctgagtactggaacccgcagaaggacgagatggaccgggtacgggccgagctggacacggtgtgcagacacaactacgggttggaagagctcaccacgttgcagcggcga

SEQ ID NO: 37 - DNA, 267 bp, Canis familiaris
gatttcgtgtaccagtttaagttcgagtgctatttcaccaacgggacggagcgggtgcggcttctggcgagagacatctataaccgggaggagttcgtgcgcttcgacagcgacgtgggggagtaccgggcggtcacggagctcgggcggccggacgctgagtactggaacgggcagaaggagctcttggagcagaggcgggccgagctggacacggtgtgcagacacaactacgggttggaagagctctacacgttgcagcggcga

SEQ ID NO: 38 - DNA, 267 bp, Canis familiaris
gatttcgtgtaccagtttaagggcgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaaccgacagaaggacgagatggaccgggtacgggccgagctggacacggtgtgcagacacaactacgggagggaagagctcaccacgttgcagcggcga

SEQ ID NO: 39 - DNA, 267 bp, Canis familiaris
gatttcgtgtaccagtttaagggcgagtgctatttcaccaacgggacggagcgggtgcggcttctgactaaatacatctataaccgggaggagttcgtgcgctttgacagcgacgtgggggagtaccgggcggtcacggagctcgggcggcccgacgctgagtactggaaccgacagaaggacgagatggaccgggtacgggccgagctggacacggtgtgcagacacaactacgggagggaagagctcaccacgttgcagcggcga

SEQ ID NO: 40 - DNA, 267 bp, Canis familiaris
gatttcgtgtaccagtttaagggcgagtgctatttcaccaacgggacggagcgggtgcggcttctgactaaatacatctataaccgggaggagttcgtgcgcttcgacagcgacgtgggggagttccgggcggtcacggagctcgggcggcccgacgctgagtactggaaccgacagaaggacgagatggaccgggtacgggccgagctggacacggtgtgcagacacaactacgggagggaagagctcaccacgttgcagcggcga

SEQ ID NO: 41 - DNA, 267 bp, Canis familiaris
gatttcgtgtaccagtttaaggccgagtgctatttcaccaacgggacggagcgggtgcggcttctgactaaatacatctataaccgggaggagcacgtgcgcttcgacagcgacgtgggggagtaccgggcggtcacggagctcgggcggccctcggctgagtactggaacccgcagaaggacgagatggaccgggtacgggccgagctggacacggtgtgcagacacaactacgggttggaagagctcaccacgttgcagcggcga

SEQ ID NO: 42 - DNA, 267 bp, Canis familiaris
gatttcgtgttccagtttaagttcgagtgctatttcaccaacgggacggagcgggtgcggcttctggcgagagacatctataaccgggaggagttcgtgcgcttcgacagcgacgtgggggagtaccgggcggtcacggagctcgggcggccggacgctgagtactggaacgggcagaaggagctcttggagcagaggcgggccgagctggacacggtgtgcagacacaactacgggttggaagagctctacacgttgcagcggcga

SEQ ID NO: 43 - DNA, 267 bp, Canis familiaris
gatttcgtgtaccagtttaagttcgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaacccgcagaaggacgagatggaccgggtacgggccgagctggacacggtgtgcagacacaactacgggagggaagagctcaccacgttgcagcggcga

SEQ ID NO: 44 - DNA, 267 bp, Canis familiaris
gatttcgtgtaccagtttaagttcgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaacccgcagaaggacgagatggaccgggtacgggccgagctggacacggtgtgcagacacaactacggggtggaagagctcaccacgttgcagcggcga

SEQ ID NO: 45 - DNA, 267 bp, Canis familiaris
gatttcgtgtaccagtttaagttcgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaacccgcagaaggacgagatggaccgggtacgggccgagctggacacggtgtgcagacacaactacggggtggaagagctctacacgttgcagcggcga

SEQ ID NO: 46 - DNA, 267 bp, Canis familiaris
gatttcgtgtaccagtttaagttcgagtgctatttcaccaacgggacggagcgggtgcggcttctgactaaatacatctataaccgggaggagttcgtgcgcttcgacagcgacgtgggggagttccgggcggtcacggagctcgggcggcccgacgctgagtactggaacccgcagaaggacgagatggaccgggtacgggccgagctggacacggtgtgcagacacaactacggggtggaagagctctacacgttgcagcggcga

SEQ ID NO: 47 - DNA, 267 bp, Canis familiaris
gatttcgtgtaccagtttaagggcgagtgctatttcaccaacgggacggagcgggtgcggcttctggcgagagacatctataaccgggaggagcacgtgcgcttcgacagcgacgtgggggagttccgggcggtcacggagctcgggcggccctgggctgagtactggaacgggcagaaggagatcttggagcggaagcgggccgagctggacacggtgtgcagacacaactacggggtggaagagctctacacgttgcagcggcga

SEQ ID NO: 48 - DNA, 267 bp, Canis familiaris
gatttcgtgtaccagtgtaaggccgagtgctatttcaccaacgggacggagcgggtgcggtttctggctaaatacatctataaccgggaggagttcgtgcgcttcgacagcgacgtgggggagttccgggcggtcacggagctcgggcggccctcggctgagtactggaacgggcagaaggagatcttggagcaggagcgggcaacggtggacacggtgtgcagacacaactacggggtggaagagctctacacgttgcagcggcga

SEQ ID NO: 49 - DNA, 267 bp, Canis familiaris
gatttcgtgtaccagtttaagggcgagtgctatttcaccaacgggacggagcgggtgcggcttctgacgagaagcatctataaccgggaggagttcgtgcgcttcgacagcgacgtgggggagttccgggcggtcacggagctcgggcggcccgtcgctgagtactggaacgggcagaaggagatcttggagcggaagcgggccgaggtggacagggtgtgcagacacaactacgggagggaagagctcaccacgttgcagcggcga

SEQ ID NO: 50 - DNA, 267 bp, Canis familiaris
gatttcgtgttccagtgtaaggccgagtgctatttcaccaacgggacggagcgggtgcggtttctggctaaatacatctataaccgggaggagttcgtgcgcttcgacagcgacgtgggggagtaccgggcggtcacggagctcgggcggcccgacgctgagtcctggaacgggcagaaggagttcttggagcaggagcgggcaacggtggacacggtgtgcagacacaactacggggtggaagagctctacacgttgcagcggcga

SEQ ID NO: 51 - DNA, 267 bp, Canis familiaris
gatttcgtgtaccagtttaagttcgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaacccgcagaaggacgagatggaccgggtgcgggccgaggtggacacggtgtgcagacacaactacggggtggaagagctctccacgttgcagcggcga

SEQ ID NO: 52 - DNA, 267 bp, Canis familiaris
gatttcgtgttccagtgtaaggccgagtgctatttcaccaacgggacggagcgggtgcggtttctggctaaatacatctataaccgggaggagttcgtgcgcttcgacagcgacgtgggggagttccgggcggtcacggagctcgggcggccctcggctgagtactggaacgggcagaaggagctcttggagcaagagcgggcaacggtggacacggtgtgcagacacaactacgggagggaagagctcaccacgttgcagcggcga

SEQ ID NO: 53 - DNA, 267 bp, Canis familiaris
gatttcgtgtaccagtttaaggccgagtgctatttcaccaacgggacggagcgggtgcggcttctgacgagaagcatctataaccgggaggagttcgtgcgcttcgacagcgacgtgggggagttccgggcggtcacggagctcgggcggcccgtcgctgagtactggaacgggcagaaggagatcttggagcggaagcgggccgcggtggacacggtgtgcagacacaactacgggagggaagagctcaccacgttgcagcggcga

SEQ ID NO: 54 - DNA, 267 bp, Canis familiaris
gatttcgtgtaccagtttaaggccgagtgctatttcaccaacgggacggagcgggtgcggcttctgacgagaagcatctataaccgggaggagttcgtgcgcttcgacagcgacgtgggggagttccgggcggtcacggagctcgggcggcccgtcgctgagtactggaacgggcagaaggagatcttggagcggaagcgggccgcggtggacagggtgtgcagacacaactacgggagggaagagctcaccacgttgcagcggcga

SEQ ID NO: 55 - DNA, 267 bp, Canis familiaris
gatttcgtgtaccagtttaaggccgagtgctatttcaccaacgggacggagcgggtgcggcttctgacgagaagcatctataaccgggaggagttcgtgcgcttcgacagcgacgtgggggagttccgggcggtcacggagctcgggcggcccgtcgctgagtactggaacgggcagaaggagatcttggagcggaagcgggccgaggtggacacggtgtgcagacacaactacgggagggaagagctcaccacgttgcagcggcga

SEQ ID NO: 56 - DNA, 267 bp, Canis familiaris
gatttcgtgtaccagtttaagggcgagtgctatttcaccaacgggacggagcgggtgcggcttctgactaaatacatctataaccgggaggagttcgtgcgcttcgacagcgacgtgggggagtaccgggcggtcacggagctcgggcggccctcggctgagtactggaacccgcagaaggacgagatggaccgggtacgggccgagctggacacggtgtgcagacacaactacgggttggaagagctcaccacgttgcagcggcga

SEQ ID NO: 57 - DNA, 267 bp, Canis familiaris
gatttcgtgtaccagtttaagggcgagtgctatttcaccaacgggacggagcgggtgcggcttctgactaaatacatctataaccgggaggagtacgtgcgcttcgacagcgacgtgggggagtaccgggcggtcacggagctcgggcggccctcggctgagtactggaacccgcagaaggacgagatggaccgggtacgggccgagctggacacggtgtgcagacacaactacgggttggaagagctcaccacgttgcagcggcga

SEQ ID NO: 58 - DNA, 267 bp, Canis familiaris
gatttcgtgtaccagtttaagggcgagtgctatttcaccaacgggacggagcgggtgcggcttctgactaaatacatctataaccgggaggagtacgtgcgcttcgacagcgacgtgggggagtaccgggcggtcacggagctcgggcggccctcggctgagtactggaacccgcagaaggacgagatggaccgggtacgggccgagctggacacggtgtgcagacacaactacggggtggaagagctctacacgttgcagcggcga

SEQ ID NO: 59 - DNA, 267 bp, Canis familiaris
gatttcgtgtaccagtgtaaggccgagtgctatttcaccaacgggacggagcgggtgcggtttctggctaaatacatctataaccgggaggagttcgtgcgcttcgacagcgacgtgggggagtaccgggcggtcacggagctcgggcggcccgacgctgagtactggaacgggcagaaggagctcttggagcaggagcgggcaacgctggacacggtgtgcagacacaactacggggtggaagagctctacacgttgcagcggcga

SEQ ID NO: 60 - DNA, 267 bp, Canis familiaris
gatttcgtgttccagtgtaaggccgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaacccgcagaaggacgagatggaccgggtacgggccgagctggacacggtgtgcagacacaactacgggttggaagagctcaccacgttgcagcggcga

SEQ ID NO: 61 - DNA, 267 bp, Canis familiaris
gatttcgtgtaccagtttaagggcgagtgctatttcaccaacgggacggagcgggtgcggcttctgacgagaagcatctataaccgggaggagttcgtgcgcttcgacagcgacgtgggggagttccgggcggtcacggagctcgggcggcccgtcgctgagtactggaacgggcagaaggagatcttggagcggaagcgggccgcggtggacagggtgtgcagacacaactacgggagggaagagctcaccacgttgcagcggcga

SEQ ID NO: 62 - DNA, 267 bp, Canis familiaris
gatttcgtgtaccagtttaagttcgagtgctatttcaccaacgggacggagcgggtgcggcttctggcgagagacatctataaccgggaggagttcgtgcgcttcgacagcgacgtgggggagtaccgggcggtcacggagctcgggcggcccgacgctgagtactggaacccgcagaaggacgagatggaccgggtacgggccgagctggacacggtgtgcagacacaactacggggtggaagagctctacacgttgcagcggcga

SEQ ID NO: 63 - DNA, 267 bp, Canis familiaris
gatttcgtgtaccagtttaagttcgagtgctatttcaccaacgggacggagcgggtgcggcttctggcgagagacatctataaccgggaggagttcgtgcgcttcgacagcgacgtgggggagtaccgggcggtcacggagctcgggcggccggacgctgagtactggaaccggcagaaggagctcttggagcagaggcgggccgcggtggacacggtgtgcagacacaactacgggttggaagagctctacacgttgcagcggcga

SEQ ID NO: 64 - DNA, 267 bp, Canis familiaris
gatttcgtgtaccagtgtaaggccgagtgctatttcaccaacgggacggagcgggtgcggtttctggctaaatacatctataaccgggaggagttcgtgcgcttcgacagcgacgtgggggagttccgggcggtcacggagctcgggcggcccgacgctgagtactggaacgggcagaaggagctcttggagcaggagcgggcaacggtggacacggtgtgcagacacaactacggggtggaagagctctacacgttgcagcggcga

SEQ ID NO: 65 - DNA, 267 bp, Canis familiaris
gatttcgtgttccagtttaagggcgagtgctatttcaccaacgggacggagcgggtgcggcttctgacgagagacatctataaccgggaggagcacgtgcgcttcgacagcgacgtgggggagtaccgggcggtcacggagctcgggcggccggacgctgagtactggaacgggcagaaggagctcttggagcggaggcgggccgaggtggacacggtgtgcagacacaactacgggagggaagagctcaccacgttgcagcggcga

SEQ ID NO: 66 - DNA, 267 bp, Canis familiaris
gatttcgtgttccagtttaagggcgagtgctatttcaccaacgggacggagcgggtgcggcttctgactaaatacatctataaccgggaggagtacgtgcgcttcgacagcgacgtgggggagtaccgggcggtcacggagctcgggcggccctcggctgagtactggaacccgcagaaggacgagatggaccgggtacgggccgagctggacacggtgtgcagacacaactacgggttggaagagctcaccacgttgcagcggcga

SEQ ID NO: 67 - DNA, 267 bp, Canis familiaris
gatttcgtgttccagtttaagttcgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaaccgacagaaggacgagatggaccgggtacgggccgagctggacacggtgtgcagacacaactacgggagggaagagctcaccacgttgcagcggcga

SEQ ID NO: 68 - DNA, 267 bp, Canis familiaris
gatttcgtgtaccagtttaaggccgagtgctatttcaccaacgggacggagcgggtgcggcttctgacgagaagcatctataaccgggaggagttggtgcgcttcgacagcgacgtgggggagttccgggcggtcacggagctcgggcggcccgtcgctgagtactggaacgggcagaaggagctcttggagcggaagcgggccgcggtggacagggtgtgcagacacaactacgggagggaagagctcaccacgttgcagcggcga

SEQ ID NO: 69 - DNA, 267 bp, Canis familiaris
gatttcgtgttccagtttaaggccgagtgctatttcaccaacgggacggagcgggtgcggcttctgacgagagacatctataaccgggaggagcacgtgcgcttcgacagcgacgtgggggagtaccgggcggtcacggagctcgggcggcccgacgctgagtactggaacgggcagaaggagctcttggagcggaggcgggccgaggtggacacggtgtgcagacacaactacgggagggaagagctcaccacgttgcagcggcga

SEQ ID NO: 70 - DNA, 267 bp, Canis familiaris
gatttcgtgtaccagtttaagttcgagtgctatttcaccaacgggacggagcgggtgcggcttctggcgagagacatctataaccgggaggagcacgtgcgcttcgacagcgacgtgggggagtaccgggcggtcacggagctcgggcggcccgacgctgagtactggaacgggcagaaggagctcttggagcagaggcgggccgagctggacacggtgtgcagacacaactacgggttggaagagctctacacgttgcagcggcga

SEQ ID NO: 71 - DNA, 267 bp, Canis familiaris
gatttcgtgttccagtataaggccgagtgctatttcaccaacgggacggagcgggtgcggcttctgactaaatacatctataaccgggaggagttcgtgcgcttcgacagcgacgtgggggagttccgggcggtcacggagctcgggcggcccgacgctgagtactggaacccgcagaaggacgagatggaccgggtacgggccgagctggacacggtgtgcagacacaactacgggagggaagagctcaccacgttgcagcggcga

SEQ ID NO: 72 - DNA, 267 bp, Canis familiaris
gatttcgtgttccagtataaggccgagtgctatttcaccaacgggacggagcgggtgcggcttctgactaaatacatctataaccgggaggagttcgtgcgcttcgacagcgacgtgggggagttccgggcggtcacggagctcgggcggcccgacgctgagtactggaacgggcagaaggacgagatggaccgggtacgggccgagctggacacggtgtgcagacacaactacgggagggaagagctcaccacgttgcagcggcga

SEQ ID NO: 73 - DNA, 267 bp, Canis familiaris
gatttcgtgtaccagtttaagggcgagtgctatttcaccaacgggacggagcgggtgcggcttctgactaaatacatctataaccgggaggagttcgtgcgcttcgacagcgacgtgggggagttccgggcggtcacggagctcgggcggccggacgctgagtactggaacgggcagaaggagctcttggagcggaggcgggccgaggtggacacggtgtgcagacacaactacgggagggaagagctcaccacgttgcagcggcga

SEQ ID NO: 74 - DNA, 267 bp, Canis familiaris
gatttcgtgttccagtttaagttcgagtgctatttcaccaacgggacggagcgggtgcggcttctggctaaatacatctataaccgggaggagttcgtgcgcttcgacagcgacgtgggggagtaccgggcggtcacggagctcgggcggccctgggctgagtactggaacccgcagaaggacgagatggaccgggtacgggccgagctggacacggtgtgcagacacaactacggggtggaagagctctacacgttgcagcggcga

SEQ ID NO: 75 - DNA, 267 bp, Canis familiaris
gatttcgtgtaccagtttaagggcgagtgctatttcaccaacgggacggagcgggtgcggcttctgactaaatacatctataaccgggaggagttggtgcgcttcgacagcgacgtgggggagtaccgggcggtcacggagctcgggcggcccgacgctgagtactggaaccgacagaaggacgagatggaccgggcacgggccgcgctggacacggtgtgcagacacaactacggggtggaagagctcaccacgttgcagcggcga

SEQ ID NO: 76 - DNA, 267 bp, Canis familiaris
gatttcgtgtaccagtgtaagtgcgagtgctatttcaccaacgggacggagcgggtgcggcttctggctaaatacatctataaccgggaggagttcgtgcgcttcgacagcgacgtgggggagtaccgggcggtcacggagctcgggcggcccttggctgagtactggaacccgcagaaggacaacatggagcaggtacgggccgagctggacacggtgtgcagacacaactacgggttggaagagctctacacgttgcagcggcga

SEQ ID NO: 77 - DNA, 267 bp, Canis familiaris
gatttcgtgtaccagtttaagggcgagtgctatttcaccaacgggacggagcgggtgcggcttctgacgagaaacatctataaccgggaggagttcgtgcgcttcgacagcgacgtgggggagttccgggcggtcacggagctcgggcggccctgggctgagtactggaacgggcagaaggagatcttggagcggaagcgggccgagctggacacggtgtgcagacacaactacgggagggaagagctcaccacgttgcagcggcga

SEQ ID NO: 78 - DNA, 267 bp, Canis familiaris
gatttcgtgttccagtttaagttcgagtgctatttcaccaacgggacggagcgggtgcggcttctgactaaatacatctataaccgggaggagtacgtgcgcttcgacagcgacgtgggggagtaccgggcggtcacggagctcgggcggccctgggctgagtactggaacccgcagaaggacgagatggaccgggtacgggccgagctggacacggtgtgcagacacaactacgggttggaagagctcaccacgttgcagcggcga

SEQ ID NO: 79 - DNA, 267 bp, Canis familiaris
gatttcgtgtaccagtttaagggcgagtgctatttcaccaacgggacggagcgggtgcggcttctggcgagagacatctataaccgggaggagcacgtgcgcttcgacagcgacgtgggggagtaccgggcggtcacggagctcgggcggccctgggctgagtactggaacgggcagaaggagatcttggagcggaagcgggccgagctggacacggtgtgcagacacaactacggggtggaagagctctacacgttgcagcggcga

SEQ ID NO: 80 - DNA, 267 bp, Canis familiaris
gatttcgtgttccagtttaagggcgagtgctatttcaccaacgggacggagcgggtgcggcttctggcgagagacatctataaccgggaggagcacgtgcgcttcgacagcgacgtgggggagtaccgggcggtcacggagctcgggcggccctgggctgagtactggaacgggcagaaggagatcttggagcggaagcgggccgagctggacacggtgtgcagacacaactacggggtggaagagctctacacgttgcagcggcga

SEQ ID NO: 81 - DNA, 267 bp, Canis familiaris
gatttcgtgtaccagtttaagggcgagtgctatttcaccaacgggacggagcgggtgcggcttctgactaaatacatctataaccgggaggagttcgtgcgcttcgacagcgacgtgggggagttccgggcggtcacggagctcgggcggcccgacgctgagtactggaacccgcagaaggacgagatggaccgggtacgggccgagctggacacggtgtgcagacacaactacgggagggaagagctcaccacgttgcagcggcga

SEQ ID NO: 82 - DNA, 267 bp, Canis familiaris
gatttcgtgtaccagtgtaagggcgagtgctatttcaccaacgggacggagcgggtgcggcttctgacgagaggcatctataaccgggaggagcacgtgcgcttcgacagcgacgtgggggagtaccgggcggtcacggagctcgggcggccctgggctgagtactggaacgggcagaaggagatcttggagcggaagcgggccgaggtggacacggtgtgcagacacaactacgggagggaagagctcaccacgttgcagcggcga

SEQ ID NO: 83 - DNA, 267 bp, Canis familiaris
gatttcgtgttccagtttaagggcgagtgctatttcaccaacgggacggagcgggtgcggcttctgactaaatacatctataaccgggaggagttcgtgcgcttcgacagcgacgtgggggagtaccgggcggtcacggagctcgggcggccctgggctgagtactggaacccgcagaaggacgagatggaccgggtacgggccgagctggacacggtgtgcagacacaactacgggttggaagagctctacacgttgcagcggcga

SEQ ID NO: 84 - DNA, 267 bp, Canis familiaris
gatttcgtgttccagtttaagttcgagtgctatttcaccaacgggacggagcgggtgcggcttctggctaaatacatctataaccgggaggagttcgtgcgcttcgacagcgacgtgggggagtaccgggcggtcacggagctcgggcggccagacgctgagtactggaacccgcagaaggacgagatggaccgggtacgggccgagctggacacggtgtgcagacacaactacggggtggaagagctctacacgttgcagcggcga

SEQ ID NO: 85 - DNA, 267 bp, Canis familiaris
gatttcgtgttccagtttaagttcgagtgctatttcaccaacgggacggagcgggtgcggcttctggctagatacatctataaccgggaggagttcgtgcgcttcgacagcgacgtgggggagtaccgggcggtcacggagctcgggcggccctacgctgagtactggaaccgacagaaggacaagatggaccgggtacgggccgagctggacacggtgtgcagacacaactacgggagggaagagctcaccacgttgcagcggcga

SEQ ID NO: 86 - DNA, 267 bp, Canis familiaris
gatttcgtgtaccagtttaagggcgagtgctatttcaccaacgggacggagcgggtgcggcttctggctaaatacatctataaccgggaggagttcgtgcgcttcgacagcgacgtgggggagtaccgggcggtcacggagctcgggcggcccgacgctgagtactggaacgggcagaaggagatcttggagcggaagcgggccgagctggacacggtgtgcagacacaactacggggtggaagagctctacacgttgcagcggcga

SEQ ID NO: 87 - DNA, 267 bp, Canis familiaris
gatttcgtgttccagtttaagggcgagtgctatttcaccaacgggacggagcgggtgcggcttctgactaaatacatctataaccgggaggagtacgtgcgcttcgacagcgacgtgggggagtaccgggcggtcacggagctcgggcggccctgggctgagtactggaacccgcagaaggacaacatggaccgggtacgggccgagctggacacggtgtgcagacacaactacgggttggaagagctctacacgttgcagcggcga

SEQ ID NO: 88 - DNA, 267 bp, Canis familiaris
gatttcgtgttccagtttaagggcgagtgctatttcaccaacgggacggagcgggtgcggcttctgactaaatacatctataaccgggaggagtacgtgcgcttcgacagcgacgtgggggagtaccgggcggtcacggagctcgggcggccctgggctgagtactggaacccgcagaaggacaagatggaccgggtacgggccgagctggacacggtgtgcagacacaactacgggttggaagagctctacacgttgcagcggcga

SEQ ID NO: 89 - DNA, 267 bp, Canis familiaris
gatttcgtgttccagtttaaggccgagtgctatttcaccaacgggacggagcgggtgcggcttctgacgagagacatctataaccgggaggagcacgtgcgcttcgacagcgacgtgggggagtaccgggcggtcacggagctcgggcggccctacgctgagtactggaacccgcagaaggagttcttggagcgggcgcgggccgaggtggacacggtgtgcagacacaactacggggtggaagagctcaccacgttgcagcggcga

SEQ ID NO: 90 - DNA, 267 bp, Canis familiaris
gatttcgtgttccagtttaaggccgagtgctatttcaccaacgggacggagcgggtgcggcttctgacgagagacatctataaccgggaggagcacgtgcgcttcgacagcgacgtgggggagtaccgggcggtcacggagctcgggcggcccgacgctgagtactggaacgggcagaaggagttcttggagcgggcgcgggccgcggtggacacggtgtgcagacacaactacggggtggaagagctcaccacgttgcagcggcga

SEQ ID NO: 91 - DNA, 267 bp, Canis familiaris
gatttcgtgttccagtttaaggcccagtgctatttcaccaacgggacggagcgggtgcggcttctgacgagagacatctataaccgggaggagcacgtgcgcttcgacagcgacgtgggggagtaccgggcggtcacggagctcgggcggcccgacgctgagtactggaacgggcagaaggagttcttggagcgggcgcgggccgcggtggacacggtgtgcagacacaactacggggtggaagagctcaccacgttgcagcggcga

SEQ ID NO: 92 - DNA, 267 bp, Canis familiaris
gatttcgtgttccagtttaaggccgagtgctatttcaccaacgggacggagcgggtgcggcttctgacgagagacatctataaccgggaggagcacgtgcgcttcgacagcgacgtgggggagtaccgggcggtcacggagctcgggcggccctacgctgagtactggaacgggcagaaggagttcttggagcgggcgcgggccgaggtggacacggtgtgcagacacaactacggggtggaagagctcaccacgttgcagcggcga

SEQ ID NO: 93 - DNA, 267 bp, Canis familiaris
gatttcgtgtaccagtttaagggcgagtgctatttcaccaacgggacggagcgggtgcggcttctggcgagaaacatctataaccgggaggagttcgtgcgcttcgacagcgacgtgggggagtaccgggcggtcacggagctcgggcggccctgggctgagtactggaacgggcagaaggagatcttggagcggaagcgggccgagctggacacggtgtgcagacacaactacggggtggaagagctcaccacgttgcagcggcga

SEQ ID NO: 94 - DNA, 267 bp, Canis familiaris
gatttcgtgttccagtttaaggccgagtgctatttcaccaacgggacggagcgggtgcggcttctgacgagagacatctataaccgggaggagcacgtgcgcttcgacagcgacgtgggggagtaccgggcggtcacggagctcgggcggccctacgctgagtactggaacgggcagaaggagttcttggagcgggcgcgggccgcggtggacacggtgtgcagacacaactacggggtggaagagctcaccacgttgcagcggcga

SEQ ID NO: 95 - DNA, 267 bp, Canis familiaris
gatttcgtgtaccagtttaagttcgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaacccgcagaaggaccagatggaccgggtacgggccgagctggacacggtgtgcagacacaactacggggtggaagagctctacacgttgcagcggcga

SEQ ID NO: 96 - DNA, 267 bp, Canis familiaris
gatttcgtgttccagtgtaagggcgagtgctatttcaccaacgggacggagcgggtgcggcttctgactaaatacatctataaccgggaggagtacgtgcgcttcgacagcgacgtgggggagtaccgggcggtcacggagctcgggcggccctgggctgagtactggaacccgcagaaggaccagatggaccgggtacgggccgagctggacacggtgtgcagacacaactacgggttggaagagctctacacgttgcagcggcga

SEQ ID NO: 97 - DNA, 267 bp, Canis familiaris
gatttcgtgtaccagtttaagggcgagtgctatttcaccaacgggacggagcgggtgcggcttctggcgagagacatctataaccgggaggagcacgtgcgcttcgacagcgacgtgggggagtaccgggcggtcacggagctcgggcggcccgacgctgagtactggaacgggcagaaggagttcttggagcgggcgcgggccgcggtggacacggtgtgcagacacaactacggggtggaagagctcaccacgttgcagcggcga

SEQ ID NO: 98 - DNA, 267 bp, Canis familiaris
gatttcgtgtwccagtttaagggcgagtgctatttcaccaacgggacggagcgggtgcggcttctgactaaatacatctataaccgggaggagtacgtgcgcttcgacagcgacgtgggggagtaccgggcggtcacggagctcgggcggccctgggctgagtactggaacccgcagaaggacgagatggaccgggtacgggccgagctggacacggtgtgcagacacaactacggggtggaagagctctacacgttgcagcggcga

SEQ ID NO: 99 - DNA, 267 bp, Canis familiaris
gatttcgtgttccagtttaagttcgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaacccgcagaaggacgagatggaccgggtacgggccgagctggacacggtgtgcagacacaactacggggtggaagagctctacacgttgcagcggcga

SEQ ID NO: 100 - DNA, 267 bp, Canis familiaris
gatttcgtgttccagtataaggccgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaacccgcagaaggacgagatggaccgggtacgggccgagctggacacggtgtgcagacacaactacggggtggaagagctctacacgttgcagcggcga

SEQ ID NO: 101 - DNA, 267 bp, Canis familiaris
gatttcgtgttccagtataaggccgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaacccgcagaaggacgagatggaccgggtacgggccgagctggacacggtgtgcagacacaactacgggagggaagagctcaccacgttgcagcggcga

SEQ ID NO: 102 - DNA, 267 bp, Canis familiaris
gatttcgtgttccagtataaggccgagtgctatttcaccaacgggacggagcgggtgcggcttctgactaaatacatctataaccgggaggagttcgtgcgcttcgacagcgacgtgggggagttccgggcggtcacggagctcgggcggcccgacgctgagtactggaaccgacagaaggacgagatggaccgggtacgggccgagctggacacggtgtgcagacacaactacggggtggaagagctctacacgttgcagcggcga

SEQ ID NO: 103 - DNA, 267 bp, Canis familiaris
gatttcgtgtaccagtttaagttcgagtgctatttcaccaacgggacggagcgggtgcggcttctgactaaatacatctataaccgggaggagttcgtgcgcttcgacagcgacgtgggggagttccgggcggtcacggagctcgggcggcccgacgctgagtactggaaccgacagaaggacgagatggaccgggtacgggccgagctggacacggtgtgcagacacaactacgggagggaagagctcaccacgttgcagcggcga

SEQ ID NO: 104 - DNA, 267 bp, Canis familiaris
gatttcgtgttccagtgtaagttcgagtgctatttcaccaacgggacggagcgggtgcggtttctggctaaatacatctataaccgggaggagttcgtgcgcttcgacagcgacgtgggggagtaccgggcggtcacggagctcgggcggcccgacgctgagtcctggaacgggcagaaggagctcttggagcaggagcgggcaacggtggacacggtgtgcagacacaactacggggtggaagagctctacacgttgcagcggcga

SEQ ID NO: 105 - DNA, 267 bp, Canis familiaris
gatttcgtgtaccagtttaagggcgagtgctatttcaccaacgggacggagcgggtgcggcttctgactaaacacatctataaccgggaggagttcgtgcgcttcgacagcgacgtgggggagttccgggcggtcacggagctcgggcggcccgacgctgagtactggaaccggcagaaggacgaggtggaccgggtacgggccgaggtggacacggtgtgcagacacaactacgggatggaggagctcaccacgttgcagcggcga

SEQ ID NO: 106 - DNA, 267 bp, Canis familiaris
gatttcgtgtaccagtttaagggcgagtgctatttcaccaacgggacggagcgggtgcggttcgtggacagatacatctataaccgggaggagttcgtgcgcttcgacagcgacgtgggggagttccgggcggtcacggagctcgggcggcccgacgctgagtactggaaccggcagaaggacgaggtggaccgggtacgggccgaggtggacacggtgtgcagacacaactacgggatggaggagctcaccacgttgcagcggcga

SEQ ID NO: 107 - DNA, 267 bp, Canis familiaris
gatttcgtgtwccagtktaagkycgagtgctatttcaccaacgggacggagcgggtgcggyttctgrctaaatacatctataaccgggaggagttcgtgcgcttcgacagcgacgtgggggagtaccgggcggtcacggagctcgggcggcccgacgctgagtmctggaacssgcagaaggaskwswtggascrggwrcgggcmrmgstggacacggtgtgcagacacaactacggggtggaagagctctacacgttgcagcggcga

SEQ ID NO: 108 - DNA, 267 bp, Canis familiaris
gatttcgtgtwccagtktaaggscgagtgctatttcaccaacgggacggagcgggtgcggyttctgrctaaatacatctataaccgggaggagttcgtgcgcttcgacagcgacgtgggggagtwccgggcggtcacggagctcgggcggccctcggctgagtactggaacssgcagaaggasswswtggascrrgwrcgggcmrmgstggacacggtgtgcagacacaactacgggwkggaagagctcaccacgttgcagcggcga

SEQ ID NO: 109 - DNA, 267 bp, Canis familiaris
gatttcgtgtaccagtgtaaggccgagtgctatttcaccaacgggacggagcgggtgcggcttctggcgagagacatctataaccgggaggagcacgtgcgcttcgacagcgacgtgggggagttccgggcggtcacggagctcgggcggccctcggctgagtactggaacgggcagaaggagctcttggagcagaggcgggccgagctggacacggtgtgcagacacaactacgggagggaagagctcaccacgttgcagcggcga

SEQ ID NO: 110 - DNA, 267 bp, Canis familiaris
gatttcgtgtaccagtgtaagggcgagtgctatttcaccaacgggacggagcgggtgcggcttctggcgagagacatctataaccgggaggagcacgtgcgcttcgacagcgacgtgggggagtaccgggcggtcacggagctcgggcggccctacgctgagtactggaacgggcagaaggagctcttggagcggaagcgggccgagctggacacggtgtgcagacacaactacgggttggaagagctcaccacgttgcagcggcga

SEQ ID NO: 111 - DNA, 267 bp, Canis familiaris
gatttcgtgttccagtttaagggcgagtgctatttcaccaacgggacggagcgggtgcggcttctgactaaatacatctataaccgggaggagttcgtgcgcttcgacagcgacgtgggggagtaccgggcggtcacggagctcgggcggcccgacgctgagtactggaacccgcagaaggacgagatggaccgggtacgggccgagctggacacggtgtgcagacacaactacgggttggaagagctctacacgttgcagcggcga

SEQ ID NO: 112 - DNA, 267 bp, Canis familiaris
gatttcgtgttccagtttaagggcgagtgctatttcaccaacgggacggagcgggtgcggcttctgactaaatacatctataaccgggaggagtacgtgcgcttcgacagcgacgtgggggagttccgggcggtcacggagctcgggcggcccgacgctgagtactggaacccgcagaaggacgagatggaccgggtacgggccgagctggacacggtgtgcagacacaactacgggttggaagagctctacacgttgcagcggcga

SEQ ID NO: 113 - DNA, 267 bp, Canis familiaris
gatttcgtgtaccagtttaagggcgagtgctatttcaccaacgggacggagcgggtgcggcttctgactaaatacatctataaccgggaggagtacgtgcgcttcgacagcgacgtgggggagttccgggcggtcacggagctcgggcggccctcggctgagtactggaacccgcagaaggacgagatggaccgggtacgggccgagctggacacggtgtgcagacacaactacgggttggaagagctcaccacgttgcagcggcga

SEQ ID NO: 114 - DNA, 270 bp, Canis familiaris
cacatttcttggaggtggcaaagtccgagtgctatttcaccaacgggacggagcgggtgcggttcgtggaaagatacatccataaccgggaggagttcgtgcgcttcgacagcgacgtgggggagtaccgggcggtcacggagctcgggcggcccgtcgctgagtcctggaacgggcagaaggagatcttggagcaggagcgggcaacggtggacacctactgcagacacaactacggggtgattgagagcttcacggtgcagcggcgag

SEQ ID NO: 115 - DNA, 270 bp, Canis familiaris
cacatttcttggaggtggcaaagtccgagtgctatttcaccaacgggacggagcgggtgcggttcgtggaaagatacatccataaccgggaggagttcgtgcgcttcgacagcgacgtgggggagttccgggcggtcacggagctcgggcggcccgtcgctgagtcctggaacgggcagaaggagatcttggagcaggagcgggcaacggtggacacctactgcagacacaactacggggtgattgagagcttcacggtgcagcggcgag

SEQ ID NO: 116 - DNA, 270 bp, Canis familiaris
cacatttcttggagatggtaaagttcgagtgccatttcaccaacgggacggagcgggtgcggtatctggcgagagacatctataaccgggaggagatcctgcgcttcgacagcgacgtgggggagtaccgggcggtcacggagctcgggcggcccatcgctgagtcctggaaccggcagaaggagatcttggagcagaggcgggccgcggtggacacctactgcagacacaactacggggtgattgagagcttcgcggtgcagcggcgag

SEQ ID NO: 117 - DNA, 270 bp, Canis familiaris
cacatttcttggagatggtaaagttcgagtgccatttcaccaacgggacggagcgggtgcggtatctggcgagagacatctataaccgggaggagatcctgcgcttcgacagcgacgtgggggagtaccgggcggtcacggagctcgggcggcccatcgctgagtcctggaaccggcagaaggagatcttggagcagaggcgggccgcggtggacacctactgcagacacaactacggggtgattgagagcttcacggtgcagcggcgag

SEQ ID NO: 118 - DNA, 270 bp, Canis familiaris
cacatttcttggaggtggcaaagtccgagtgctatttcaccaacgggacggagcgggtgcggttcgtggaaagatacatccataaccgggaggagaacgtgcgcttcgacagcgacgtgggggagttccgggcggtcacggagctcgggcggcccgacgctgagtcctggaaccggcagaaggagctcttggagcggaagcgggccgcggtggacacctactgcagacacaactacggggtgattgagagcttcacggtgcagcggcgag

SEQ ID NO: 119 - DNA, 270 bp, Canis familiaris
cacatttcgtgtaccagtttaagcccgagtgccatttcaccaacgggacggagcgggtgcggttcgtggaaagacacatccataaccgggaggagttcgtgcgcttcgacagcgacgtgggggagtaccgggcggtcacggagctcgggcggcccgacgctgagtcctggaacgggcagaaggagctcttggagcaggagcgggcaacggtggacacctactgcagacacaactacggggtgattgagagcttcacggtgcagcggcgag

SEQ ID NO: 120 - DNA, 270 bp, Canis familiaris
cacatttcttggagatgttaaagttcgagtgccatttcaccaacgggacggagcgggtgcggttcgtggaaagatacatccataaccgggaggagaacgtgcgcttcgacagcgacgtgggggagtaccgggcggtcacggagctcgggcggcccgacgctgagtcctggaaccggcagaaggagctcttggagcaggagcgggccgcggtggacacctactgcagacacaactaccgggtgggcgagagcttcacggtgcagcggcgag

SEQ ID NO: 121 - DNA, 270 bp, Canis familiaris
cacatttcttggaggtggcaaaggccgagtgccatttcaccaacgggacggagcgggtgcggttcgtggaaagatacatctataaccgggaggagtacgtgcgcttcgacagcgacgtgggggagtaccgggcggtcacggagctcgggcggcccgacgctgagtactggaacccgcagaaggagctcttggagcgggcgcgggccgcggtggacacctactgcagacacaactacggggtgggcgagagcttcacggtgcagcggcgag

SEQ ID NO: 122 - DNA, 270 bp, Canis familiaris
cacatttcttggaggtggcaaaggccgagtgccatttcaccaacgggacggagcgggtgcggttcgtggaaagatacatctataaccgggaggagttcgtgcgcttcgacagcgacgtgggggagtaccgggcggtcacggagctcgggcggcccgacgctgagtactggaacccgcagaaggagctcttggaggggggccgggccgcggtggacacctactgcagacacaactacggggtgggcgagagcttcacggtgcagcggcgag

SEQ ID NO: 123 - DNA, 270 bp, Canis familiaris
cacatttcgtgaagatgtataaggccgagtgccatttcaccaacgggacggagcgggtgcggtatctgatgagagacatctataaccgggaggagttcgtgcgcttcgacagcgacgtgggggagttccgggcggtcacggagctcgggcggcgcgacgctgagtactggaacgggcagaaggagatcttggagcggaagcgggccgcggtggacacctactgcagacacaactaccgggtgggcgagagcttcacggtgcagcggcgag

SEQ ID NO: 124 - DNA, 270 bp, Canis familiaris
cacatttcgtgaagatgtataaggccgagtgccatttcaccaacgggacggagcgggtgcggtatctgatgagagacatctataaccgggaggagttcgtgcgcttcgacagcgacgtgggggagttccgggcggtcacggagctcgggcggcgcgacgctgagtactggaacgggcagaaggagatcttggagcggaagcgggccgcggtggacacctactgcagacacaactacggggtgattgagagcttcgcggtgcagcggcgag

SEQ ID NO: 125 - DNA, 270 bp, Canis familiaris
cacatttcttggaggtggcaaagtccgagtgctatttcaccaacgggacggagcgggtgcggttcgtggaaagatacatccataaccgggaggagaacgtgcgcttcgacagcgacgtgggggagtaccgggcggtcacggagctcgggcggcccgacgctgagtcctggaaccggcagaaggagttcttggagcggaagcgggccgcggtggacacctactgcagacacaactacggggtgattgagagcttcacggtgcagcggcgag

SEQ ID NO: 126 - DNA, 270 bp, Canis familiaris
cacatttcgtgtaccagtttaagcccgagtgccatttcaccaacgggacggagcgggtgcggttcgtggaaagatacatccataaccgggaggagttcgtgcgcttcgacagcgacgtgggggagtaccgggcggtcacggagctcgggcggcccgtcgctgagtcctggaacgggcagaaggagctcttggagcaggagcgggcaacggtggacacctactgcagacacaactacggggtgattgagagcttcacggtgcagcggcgag

SEQ ID NO: 127 - DNA, 270 bp, Canis familiaris
cacatttcgtgtaccagtttaagcccgagtgccatttcaccaacgggacggagcgggtgcggttcgtggaaagatacatccacaaccgggaggagttcgtgcgcttcgacagcgacgtgggggagtaccgggcggtcacggagctcgggcggcccgtcgctgagtcctggaacgggcagaaggagctcttggagcaggagcgggcaacggtggacacctactgcagacacaactacggggtgattgagagcttcrcggtgcagcggcgag

SEQ ID NO: 128 - DNA, 270 bp, Canis familiaris
cacatttcgtgaagatgtttaaggccgagtgccatttcaccaacgggacggagcgggtgcggcttctggcgagaagcatctataaccgggaggagttcgtgcgcttcgacagcgacgtgggggagttccgggcggtcacggagctcgggcggcgcgacgctgagtcctggaaccggcagaaggagctcttggagcagaggcgggccgcggtggacacctactgcagacacaactaccgggtgggcgagagcttcacggtgcagcggcgag

SEQ ID NO: 129 - DNA, 270 bp, Canis familiaris
cacatttcgtgaggatgtataaggccgagtgccatttcaccaacgggacggagcgggtgcggtttctggcgagaagcatctataaccgggaggagttcgcgcgcttcgacagcgacgtgggggagtaccgggcggtcacggagctcgggcggcgcgacgctgagtcctggaaccggcagaaggagctcttggagcggaggcgggccgaggtggacacctactgcagacacaactacggggtgattgagagcttcacggtgcagcggcgag

SEQ ID NO: 130 - DNA, 270 bp, Canis familiaris
cacatttcgtgtaccagtttaagcccgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaaccggcagaaggagctcttggagcaggagcgggccgcggtggacacctactgcagacacaactaccgggtgggcgagagcttcacggtgcagcggcgag

SEQ ID NO: 131 - DNA, 270 bp, Canis familiaris
cacatttcgtgtaccagtttaagcccgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaaccggcagaaggagctcttggagcaggagcgggccgcggtggacacggtgtgcagacacaactaccgggtgggcgagagcttcacggtgcagcggcgag

SEQ ID NO: 132 - DNA, 270 bp, Canis familiaris
cacatttcttggagatgttaaagtccgagtgctatttcaccaacgggacggagcgggtgcggttcgtggaaagatacatccataaccgggaggagaacgtgcgcttcgacagcgacgtgggggagtaccgggcggtcacggagctcgggcggcccgacgctgagtactggaacccgcagaaggagctcttggagcgggcgcgggccgcggtggacacctactgcagacacaactacggggtgggcgagagcttcacggtgcagcggcgag

SEQ ID NO: 133 - DNA, 270 bp, Canis familiaris
cacatttcttggagatggtaaagttcgagtgccatttcaccaacgggacggagcgggtgcggcttctggtgagagacatctataaccgggaggagcacgtgcgcttcgacagcgacgtgggggagtaccgggcggtcacggagctcgggcggcccgacgctgagtactggaacgggcagaaggagctcttggagcagaggcgggccgaggtggacacggtgtgcagacacaactacggggtgattgagagcttcacggtgcagcggcgag

SEQ ID NO: 134 - DNA, 270 bp, Canis familiaris
cacatttcttggagatggtaaagttcgagtgccatttcaccaacgggacggagcgggtgcggcttctggtgagagacatctataaccgggaggagcacgtgcgcttcgacagcgacgtgggggagtaccgggcggtcacggagctcgggcggcccgacgctgagtactggaacgggcagaaggagctcttggagcagaggcgggccgaggtggacacggtgtgcagacacaactacggggtgattgagagcttcgcggtgcagcggcgag

SEQ ID NO: 135 - DNA, 270 bp, Canis familiaris
cacatttcttggagatggtaaagttcgagtgccatttcaccaacgggacggagcgggtgcggcttctggtgagagacatctataaccgggaggagcacgtgcgcttcgacagcgacgtgggggagtaccgggcggtcacggagctcgggcggcccgacgctgagtactggaacgggcagaaggagctcttggagcagaggcgggccgaggtggacacctactgcagacacaactacggggtgattgagagcttcacggtgcagcggcgag

SEQ ID NO: 136 - DNA, 270 bp, Canis familiaris
cacatttcttggagatggtaaagttcgagtgccatttcaccaacgggacggagcgggtgcggcttctggtgagagacatctataaccgggaggagcacgtgcgcttcgacagcgacgtgggggagttccgggcggtcacggagctcgggcggcccgacgctgagtactggaacgggcagaaggagctcttggagcagaggcgggccgaggtggacacggtgtgcagacacaactacggggtgattgagagcttcacggtgcagcggcgag

SEQ ID NO: 137 - DNA, 270 bp, Canis familiaris
cacatttcttggaggtggcaaaggccgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaaccggcagaaggagctcttggagcggaagcgggccgaggtggacacctactgcagacacaactacggggtgattgagagcttcacggtgcagcggcgag

SEQ ID NO: 138 - DNA, 270 bp, Canis familiaris
cacatttcgtgaagatgtttaaggccgagtgccatttcaccaacgggacggagcgggtgcggtttctggcgagaagcatctataaccgggaggagttcgtgcgcttcgacagcgacgtgggggagtaccgggcggtcacggagctcgggcggcgcgacgctgagtcctggaaccggcagaaggagctcttggagcgggcgcgggccgcggtggacacctactgcagacacaactacggggtgattgagagcttcacggtgcagcggcgag

SEQ ID NO: 139 - DNA, 270 bp, Canis familiaris
cacatttcgtgaagatgtttaaggccgagtgccatttcaccaacgggacggagcgggtgcggtttctggcgagaagcatctataaccgggaggagttcgtgcgcttcgacagcgacgtgggggagtaccgggcggtcacggagctcgggcggcgcgacgctgagtcctggaaccggcagaaggagctcttggagcgggcgcgggccgcggtggacacctactgcagacacaactaccgggtgggcgagagcttcacggtgcagcggcgag

SEQ ID NO: 140 - DNA, 270 bp, Canis familiaris
cacatttcttggaggtggcaaagtccgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaaccggcagaaggagctcttggagcggaagcgggccgaggtggacacctactgcagacacaactacggggtgattgagagcttcacggtgcagcggcgag

SEQ ID NO: 141 - DNA, 270 bp, Canis familiaris
cacatttcgtgaggatgtataaggccgagtgccatttcaccaacgggacggagcgggtgcggtttctggcgagaagcatctataaccgggaggagttcgtgcgcttcgacagcgacgtgggggagtaccgggcggtcacggagctcgggcggcgcgacgctgagtcctggaaccggcagaaggagctcttggagcggaggcgggccgaggtggacacctactgcagacacaactacggggtgattgagagcttcacggtgcagcggcgag

SEQ ID NO: 142 - DNA, 270 bp, Canis familiaris
cacatttcttgaagatggtaaagttcgagtgccatttcaccaacgggacggagcgggtgcggttggtggaaagagacatctataaccgggaggagtacgtgcgcttcgacagcgacgtgggggagtaccgggcggtcacggagctcgggcggccctcggctgagtcctggaaccggcagaaggagttcttggagcagaggcgggccgaggtggacacggtgtgcagacacaactacggggtgggcgagagcttcacggtgcagcggcgag

SEQ ID NO: 143 - DNA, 270 bp, Canis familiaris
cacatttcgtgaagatgtttaaggccgagtgccatttcaccaacgggacggagcgggtgcggcttctggcgagaagcatctataaccgggaggagttcgtgcgcttcgacagcgacgtgggggagttccgggcggtcacggagctcgggcggcgcgacgctgagtcctggaaccggcagaaggagctcttggagcagaggcggcccgcggtggacacctactgcagacacaactaccgggtgggcgagagcttcacggtgcagcggcgag

SEQ ID NO: 144 - DNA, 270 bp, Canis familiaris
cacatttcgtgtaccagtttaagcccgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaacgggcagaaggagatcttggagcaggagcgggccgaggtggacacctactgcagacacaactacggggtgattgagagcttcacggtgcagcggcgag

SEQ ID NO: 145 - DNA, 270 bp, Canis familiaris
cacatttcttggagatgttaaagttcgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaaccggcagaaggagctcttggagcaggagcgggccgcggtggacacctactgcagacacaactaccgggtgggcgagagcttcacggtgcagcggcgag

SEQ ID NO: 146 - DNA, 270 bp, Canis familiaris
cacatttcttggaggtgttaaagtccgagtgctatttcaccaacgggacggagcgggtgcggttcgtggaaagatacatccataaccgggaggagaacgtgcgcttcgacagcgacgtgggggagtaccgggcggtcacggagctcgggcggcgcgacgctgagtcctggaaccggcagaaggagctcttggagcggaagcgggccgaggtggacacctactgcagacacaactaccgggtgggcgagagcttcacggtgcagcggcgag

SEQ ID NO: 147 - DNA, 270 bp, Canis familiaris
cacatttcttggaggtgttaaagttcgagtgccatttcaccaacgggacggagcgggtgcggttggtggaaagatacatctataaccgggaggagttcgcgcgcttcgacagcgacgtgggggagtaccgggcggtcacggagctcgggcggcgcgacgctgagtcctggaaccggcagaaggagctcttggagcagaggcgggccgcggtggacacctactgcagacacaactaccgggtgggcgagagcttcacggtgcagcggcgag

SEQ ID NO: 148 - DNA, 270 bp, Canis familiaris
cacatttcttggagatgttaaagtccgagtgctatttcaccaacgggacggagcgggtgcggttcgtggaaagatacatccataaccgggaggagaacgtgcgcttcgacagcgacgtgggggagtaccgggcggtcacggagctcgggcggcccgacgctgagtactggaaccggcagaaggagctcttggagcggaagcgggccgaggtggacacctactgcagacacaactacggggtgattgagagcttcacggtgcagcggcgag

SEQ ID NO: 149 - DNA, 270 bp, Canis familiaris
cacatttcgtgtaccagtttaaggccgagtgccatttcaccaacgggacggagcgggtgcggttcgtggaaagatacatccataaccgggaggagttcgtgcgcttcgacagcgacgtgggggagtaccgggcggtcacggagctcgggcggcgcgacgctgagtcctggaacgggcagaaggagatcttggagcaggagcgggcaacggtggacacctactgcagacacaactacggggtgattgagagcttcacggtgcagcggcgag

SEQ ID NO: 150 - DNA, 270 bp, Canis familiaris
cacatttcttggaggtggcaaaggccgagtgccatttcaccaacgggacggagcgggtgcggttcgtggaaagatacatctataaccgggaggagttcgtgcgcttcgacagcgacgtgggggagtaccgggcggtcacggagctcgggcggcccgacgctgagtactggaacccgcagaaggagctcttggagcgggcgcgggccgcggtggacacctactgcagacacaactacggggtgggcgagagcttcacggtgcagcggcgag

SEQ ID NO: 151 - DNA, 270 bp, Canis familiaris
cacatttcgtgaagatgtataaggccgagtgccatttcaccaacgggacggagcgggtgcggtatctgatgagagacatctataaccgggaggagaacgtgcgcttcgacagcgacgtgggggagttccgggcggtcacggagctcgggcggcgcgacgctgagtcctggaacgggcagaaggagatcttggagcggaagcgggccgcggtggacacctactgcagacacaactacggggtgattgagagcttcgcggtgcagcggcgag

SEQ ID NO: 152 - DNA, 270 bp, Canis familiaris
cacatttcttggagatggtaaagttcgagtgccatttcaccaacgggacggagcgggtgcggcttctggtgagagacatctataaccgggaggagcacgtgcgcttcgacagcgacgtgggggagtaccgggcggtcacggagctcgggcggcccgacgctgagtcctggaaccggcagaaggagctcttggagcagaggcgggccgaggtggacacggtgtgcagacacaactacggggtgattgagagcttcrcggtgcagcggcgag

SEQ ID NO: 153 - DNA, 270 bp, Canis familiaris
cacatttcttgaagatggtaaagttcgagtgccatttcaccaacgggacggagcgggtgcggtatctgatgagagacatctataaccgggaggagttcgtgcgcttcgacagcgacgtgggggagttccgggcggtcacggagctcgggcggcgcgacgctgagtcctggaacgggcagaaggagctcttggagcagaagcgggccgcggtggacacctactgcagacacaactacggggtgattgagagcttcgcggtgcagcggcgag

SEQ ID NO: 154 - DNA, 270 bp, Canis familiaris
cacatttcgtgtaccagtttaagcccgagtgccatttcaccaacgggacggagcgggtgcggttcgtggaaagatacatccataaccgggaggagttcgtgcgcttcgacagcgacgtgggggagtaccgggcggtcacggagctcgggcggcccgtcgctgagtcctggaacgggcagaaggagatcttggagcaggagcgggcaacggtggacacggtgtgcagacacaactacggggtgattgagagcttcacggtgcagcggcgag

SEQ ID NO: 155 - DNA, 270 bp, Canis familiaris
cacatttcgtgtaccagtttaagcccgagtgccatttcaccaacgggacggagcgggtgcggttcgtggaaagatacatccataaccgggaggagttcgtgcgcttcgacagcgacgtgggggagtaccgggcggtcacggagctcgggcggcccgtcgctgagtcctggaacgggcagaaggagatcttggagcaggagcgggcaacggtggacacctactgcagacacaactacggggtgattgagagcttcacggtgcagcggcgag

SEQ ID NO: 156 - DNA, 270 bp, Canis familiaris
cacatttcttggagatgttaaagttcgagtgccatttcaccaacgggacggagcgggtgcggtatctggtgagagacatctataaccgggaggagcacgtgcgcttcgacagcgacgtgggggagtaccgggcggtcacggagctcgggcggcccgacgctgagtactggaaccggcagaaggagctcttggagcggaggcgggccgaggtggacacctactgcagacacaactaccgggtgggcgagagcttcacggtgcagcggcgag

SEQ ID NO: 157 - DNA, 270 bp, Canis familiaris
cacatttcgtgaagatgtttaaggccgagtgccatttcaccaacgggacggagcgggtgcggcttctggcgagaagcatctataaccgggaggagttcgtgcgcttcgacagcgacgtgggggagttccgggcggtcacggagctcgggcggcgcgacgctgagtcctggaaccggcagaaggagttcttggagcagaggcgggccgcggtggacacctactgcagacacaactaccgggtgggcgagagcttcacggtgcagcggcgag

SEQ ID NO: 158 - DNA, 270 bp, Canis familiaris
cacatttcttggagatgttaaagtccgagtgccatttcaccaacgggacggagcgggtgcggttcgtggaaagatacatccataaccgggaggagaacgtgcgcttcgacagcgacgtgggggagtaccgggcggtcacggagctcgggcggcgcgacgctgagtcctggaaccggcagaaggagctcttggagcggaagcgggccgaggtggacacctactgcagacacaactacggggtgattgagagcttcacggtgcagcggcgag

SEQ ID NO: 159 - DNA, 270 bp, Canis familiaris
cacatttcttggaggtggcaaaggccgagtgccatttcaccaacgggacggagcgggtgcggttcgtggaaagatacatctataaccgggaggagtacgtgcgcttcgacagcgacgtgggggagtaccgggcggtcacggagctcgggcggcccgacgctgagtcctggaacccgcagaaggagctcttggagcgggcgcgggccgcggtggacacctactgcagacacaactacggggtgggcgagagcttcacggtgcagcggcgag

SEQ ID NO: 160 - DNA, 270 bp, Canis familiaris
cacatttcttggagatggtaaagttcgagtgccatttcaccaacgggacggagcgggtgcggcttctggtgagagacatctataaccgggaggagcacgtgcgcttcgacagcgacgtgggggagtaccgggcggtcacggagctcgggcggcccgacgctgagtactggaacgggcagaaggagctcttggagcggaggcgggccgaggtggacacggtgtgcagacacaactaccgggtgattgagagcttcacggtgcagcggcgag

SEQ ID NO: 161 - DNA, 270 bp, Canis familiaris
cacatttcttgaagatggtaaagttcgagtgccatttcaccaacgggacggagcgggtgcggttggtggaaagagacatctataaccgggaggagtacgtgcgcttcgacagcgacgtgggggagttccgggcggtcacggagctcgggcggccctcggctgagtactggaacgggcagaaggagatcttggagcagaggcgggccgaggtggacacggtgtgcagacacaactacggggtgattgagagcttcgcggtgcagcggcgag

SEQ ID NO: 162 - DNA, 270 bp, Canis familiaris
cacatttcttggagatgttaaagttcgagtgccatttcaccaacgggacggagcgggtgcggtatctgatgagagacatctataaccgggaggagaacgtgcgcttcgacagcgacgtgggggagttccgggcggtcacggagctcgggcggcgcgacgctgagtcctggaacgggcagaaggagctcttggagcagaagcgggccgcggtggacacctactgcagacacaactacggggtgattgagagcttcgcggtgcagcggcgag

SEQ ID NO: 163 - DNA, 270 bp, Canis familiaris
cacatttcttggagatgttaaagttcgagtgccatttcaccaacgggacggagcgggtgcggtatctggtgagagacatctataaccgggaggagaacgtgcgcttcgacagcgacgtgggggagttccgggcggtcacggagctcgggcggcccgacgctgagtactggaacgggcagaaggagctcttggagcagaagcgggccgcggtggacacctactgcagacacaactacggggtgattgagagcttcgcggtgcagcggcgag

SEQ ID NO: 164 - DNA, 270 bp, Canis familiaris
cacatttcttggagatgttaaagttcgagtgccatttcaccaacgggacggagcgggtgcggtatctggtgagagacatctataaccgggaggagcacgtgcgcttcgacagcgacgtgggggagtaccgggcggtcacggagctcgggcggcccgacgctgagtactggaaccggcagaaggagctcttggagcggaggcgggccgaggtggacacggtgtgcagacacaactaccgggtgggcgagagcttcacggtgcagcggcgag

SEQ ID NO: 165 - DNA, 270 bp, Canis familiaris
cacatttcttggaggtggcaaagtccgagtgctatttcaccaacgggacggagcgggtgcggttagtggaaagatacatccataaccgggaggagaacgtgcgcttcgacagcgacgtgggggagttccgggcggtcacggagctcgggcggcccgacgctgagtcctggaaccggcagaaggagctcttggagcagaggcgggccgcggtggacacctactgcagacacaactaccgggtgggcgagagcttcacggtgcagcggcgag

SEQ ID NO: 166 - DNA, 270 bp, Canis familiaris
cacatttcttggagatgttaaagtccgagtgctatttcaccaacgggacggagcgggtgcggttcgtggaaagatacatccataaccgggaggagaacgtgcgcttcgacagcgacgtgggggagtaccgggcggtcacggagctcgggcggcccgacgctgagtcctggaacgggcagaaggagctcttggagcggaagcgggccgaggtggacacctactgcagacacaactacggggtgggcgagagcttcacggtgcagcggcgag

SEQ ID NO: 167 - DNA, 270 bp, Canis familiaris
cacatttcttggagatgttaaagtccgagtgctatttcaccaacgggacggagcgggtgcggttcgtggaaagatacatccataaccgggaggagaacgtgcgcttcgacagcgacgtgggggagtaccgggcggtcacggagctcgggcggcccgacgctgagtcctggaacgggcagaaggagctcttggagcggaagcgggccgaggtggacacctactgcagacacaactacggggtgattgagagcttcacggtgcagcggcgag

SEQ ID NO: 168 - DNA, 270 bp, Canis familiaris
cacatttcttggagatgttaaagtccgagtgctatttcaccaacgggacggagcgggtgcggttcgtggaaagatacatccataaccgggaggagaacgtgcgcttcgacagcgacgtgggggagtaccgggcggtcacggagctcgggcggcccgacgctgagtcctggaaccggcagaaggagctcttggagcaggagcgggccgcggtggacacctactgcagacacaactacggggtgattgagagcttcacggtgcagcggcgag

SEQ ID NO: 169 - DNA, 270 bp, Canis familiaris
cacatttcttgaagatggtaaagttcgagtgccatttcaccaacgggacggagcgggtgcggtatctgatgagagacatctataaccgggaggagttcgtgcgcttcgacagcgacgtgggggagttccgggcggtcacggagctcgggcggcgcgacgctgagtcctggaacgggcagaaggagctcttggagcggaggcgggccgcggtggacacctactgcagacacaactacggggtgattgagagcttcacggtgcagcggcgag

SEQ ID NO: 170 - DNA, 270 bp, Canis familiaris
cacatttcttggagatgttaaagtccgagtgctatttcaccaacgggacggagcgggtgcggttcgtggaaagatacatccataaccgggaggagaacgtgcgcttcgacagcgacgtgggggagtaccgggcggtcacggagctcgggcggcccgacgctgagtcctggaaccggcagaaggagctcttggagcggaagcgggccgaggtggacacctactgcagacacaactacggggtgattgagagcttcacggtgcagcggcgag

SEQ ID NO: 171 - DNA, 270 bp, Canis familiaris
cacatttcttgaagatggtaaagttcgagtgccatttcaccaacgggacggagcgggtgcggttggtggaaagagacatctataaccgggaggagtacgtgcgcttcgacagcgacgtgggggagttccgggcggtcacggagctcgggcggccctcggctgagtactggaacgggcagaaggagctcttggagcagaggcgggccgaggtggacacggtgtgcagacacaactacggggtgattgagagcttcgcggtgcagcggcgag

SEQ ID NO: 172 - DNA, 270 bp, Canis familiaris
cacatttcttggagatggtaaagtccgagtgccatttcaccaacgggacggagcgggtgcggcttctggtgagagacatctataaccgggaggagcacgtgcgcttcgacagcgacgtgggggagtaccgggcggtcacggagctcgggcggcccgacgctgagtactggaacgggcagaaggagctcttggagcagaggcgggccgaggtggacacggtgtgcagacacaactacggggtgattgagagcttcgcggtgcagcggcgag

SEQ ID NO: 173 - DNA, 270 bp, Canis familiaris
cacatttcgtgtaccagtttaaggccgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaacgggcagaaggagatcttggagcaggagcgggcaacggtggacacctactgcagacacaactacggggtgattgagagcttcacggtgcagcggcgag

SEQ ID NO: 174 - DNA, 270 bp, Canis familiaris
cacatttcttggagatggtaaagttcgagtgccatttcaccaacgggacggagcgggtgcggtatctgctgagagacatctataaccgggaggagatcctgcgcttcgacagcgacgtgggggagttccgggcggtcacggagctcgggcggcccgacgctgagtcctggaaccggcagaaggagctcttggagcagaagcgggccgcggtggacacctactgcagacacaactaccgggtgggcgagagcttcacggtgcagcggcgag

SEQ ID NO: 175 - DNA, 270 bp, Canis familiaris
cacatttcttgaagatggtaaagttcgagtgccatttcaccaacgggacggagcgggtgcggtatctgatgagagacatctataaccgggaggagttcgtgcgcttcgacagcgacgtgggggagtaccgggcggtcacggagctcgggcggcccgacgctgagtcctggaacgggcagaaggagctcttggagcaggagcgggcaacggtggacacctactgcagacacaactaccgggtgggcgagagcttcacggtgcagcggcgag

SEQ ID NO: 176 - DNA, 270 bp, Canis familiaris
cacatttcttggaggtggcaaagtccgagtgctatttcaccaacgggacggagcgggtgcggttcgtggaaagatacatccataaccgggaggagaacgtgcgcttcgacagcgacgtgggggagtaccgggcggtcacggagctcgggcggcccgacgctgagtcctggaacgggcagaaggagctcttggagcagaggcgggccgcggtggacacctactgcagacacaactacggggtgattgagagcttcacggtgcagcggcgag

SEQ ID NO: 177 - DNA, 270 bp, Canis familiaris
cacatttcttggagatgtataaggccgagtgccatttcaccaacgggacggagcgggtgcggtttctggcgagaagcatctataaccgggaggagttcgtgcgcttcgacagcgacgtgggggagtaccgggcggtcacggagctcgggcggcccatcgctgagtcctggaaccggcagaaggagttcttggagcggaggcgggccgaggtggacacctactgcagacacaactacggggtgattgagagcttcacggtgcagcggcgag

SEQ ID NO: 178 - DNA, 270 bp, Canis familiaris
cacatttcttggaggtggcaaaggccgagtgctatttcaccaacgggacggagcgggtgcggttcgtggaaagatacatccataaccgggaggagaacgtgcgcttcgacagcgacgtgggggagtaccgggcggtcacggagctcgggcggcccgacgctgagtcctggaacgggcagaaggagctcttggagcggaagcgggccgaggtggacacctactgcagacacaactacggggtgggcgagagcttcacggtgcagcggcgag

SEQ ID NO: 179 - DNA, 270 bp, Canis familiaris
cacatttcgtgtaccagtttaagcccgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtactggaacgggcagaaggagatcttggagcaggagcgggcaacggtggacacctactgcagacacaactacggggtgattgagagcttcacggtgcagcggcgag

SEQ ID NO: 180 - DNA, 250 bp, Canis familiaris
cacatttcgtgtaccagtttaagcccgagtgccatttcaccaacgggacggagcgggtgcggttcgtggaaagatacatccataaccgggaggagatcctgcgcttcgacagcgacgtgggggagtaccgggcggtcacggagctcgggcggcccgtcgctgagtcctggaacgggcagaaggagatcttggagcaggagcgggcaacggtggacacggtgtgcagacacaactacggggtgattgagag

SEQ ID NO: 181 - DNA, 270 bp, Canis familiaris
cacatttcgtgaagatgtttaaggccgagtgccatttcaccaacgggacggagcgggtgcggcttctggcgagaagcatctataaccgggaggagttcgtgcgcttcgacagcgacgtgggggagtaccgggcggtcacggagctcgggcggcccgacgctgagtcctggaaccggcagaaggagctcttggagcagaggcgggccgaggtggacacctactgcagacacaactacggggtgattgagagcttcacggtgcagcggcgag

SEQ ID NO: 182 - DNA, 270 bp, Canis familiaris
cacatttcgtgtaccagtttaagcccgagtgccatttcaccaacgggacggagcgggtgcggttcgtggaaagatacatccataaccgggaggagttcgtgcgcttcgacagcgacgtgggggagttccgggcggtcacggagctcgggcggccctcggctgagtcctggaacgggcagaaggagctcttggagcagaagcgggccgcggtggacacctactgcagacacaactacggggtgattgagagcttcgcggtgcagcggcgag

SEQ ID NO: 183 - DNA, 270 bp, Canis familiaris
cacatttcttggagatgttaaagttcgagtgccatttcaccaacgggacggagcgggtgcggtatctgatgagagacatctataaccgggaggagcacgtgcgcttcgacagcgacgtgggggagttccgggcggtcacggagctcgggcggcccgacgctgagtactggaaccggcagaaggagctcttggagcggaggcgggccgaggtggacacggtgtgcagacacaactacggggtgattgagagcttcacggtgcagcggcgag

SEQ ID NO: 184 - DNA, 270 bp, Canis familiaris
cacatttcttggagatgttaaagttcgagtgccatttcaccaacgggacggagcgggtgcggtatctgatgagagacatctataaccgggaggagcacgtgcgcttcgacagcgacgtgggggagttccgggcggtcacggagctcgggcggcccgacgctgagtactggaacgggcagaaggagctcttggagcggaagcgggccgaggtggacacggtgtgcagacacaactacggggtgattgagagcttcacggtgcagcggcgag

SEQ ID NO: 185 - DNA, 270 bp, Canis familiaris
cacatttcttggagatgtttaagttcgagtgctatttcaccaacgggacggagcgggtgcggcttctggtgagaagcatctataaccgggaggagttcgtgcgcttcgacagcgacgtgggggagtaccgggcggtcacggagctcgggcggcccatcgctgagtcctggaaccggcagaaggagttcttggagcagaggcgggccgaggtggacacggtgtgcagacacaactacggggtgggcgagagcttcacggtgcagcggcgag

SEQ ID NO: 186 - DNA, 270 bp, Canis familiaris
cacatttcgtgaggatgtttaaggccgagtgccatttcaccaacgggacggagcgggtgcggtttctggcgagaagcatctataaccgggaggagttcgtgcgcttcgacagcgacgtgggggagtaccgggcggtcacggagctcgggcggcccgacgctgagtactggaaccggcagaaggagctcttggagcagaagcgggccgcggtggacacctactgcagacacaactaccgggtgggcgagagcttcacggtgcagcggcgag

SEQ ID NO: 187 - DNA, 270 bp, Canis familiaris
cacatttcttggagatgttaaagtccgagtgccatttcaccaacgggacggagcgggtgcggcttctggtgagagacatctataaccgggaggagttggtgcgcttcgacagcgacgtgggggagttccgggcggtcacggagctcgggcggcccatcgctgagtcctggaaccggcagaaggagctcttggagcggaggcgggccgaggtggacacggtgtgcagacacaactacggggtgattgagagcttcacggtgcagcggcgag

SEQ ID NO: 188 - DNA, 270 bp, Canis familiaris
cacatttcttggagatgttaaagtccgagtgccatttcaccaacgggacggagcgggtgcggtatctggtgagagacatctataaccgggaggagcacgtgcgcttcgacagcgacgtgggggagtaccgggcggtcacggagctcgggcggcccgacgctgagtcctggaaccggcagaaggagctcttggagcggaggcgggccgaggtggacacctactgcagacacaactacggggtgattgagagcttcacggtgcagcggcgag

SEQ ID NO: 189 - DNA, 270 bp, Canis familiaris
cacatttcttggagatggtaaagttcgagtgccatttcaccaacgggacggagcgggtgcggcttctggtgagagacatctataaccgggaggagcacgtgcgcttcgacagcgacgtgggggagtaccgggcggtcacggagctcgggcggcccgacgctaagtactacaacgggcagaaggagctcttggagcagaggcgggccgaggtggacacggtgtgcaaacacaactacggggtgattgagagcttcacggtgcagcggcgag

SEQ ID NO: 190 - DNA, 270 bp, Canis familiaris
cacatttcttggagatgttaaagtccgagtgctatttcaccaacgggacggagcgggtgcggttcgtggaaagatacatccataaccgggaggagcacgtgcgcttcgacagcgacgtgggggagtaccgggcggtcacggagctcgggcggcccgacgctgagtcctggaaccggcagaaggagctcttggagcggaagcgggccgcggtggacacctactgcagacacaactacggggtgattgagagcttcgcggtgcagcggcgag

SEQ ID NO: 191 - DNA, 270 bp, Canis familiaris
cacatttcgtgaagatgtttaaggccgagtgccatttcaccaacgggacggagcgggtgcggcttctggcgagaagcatctataaccgggaggagttcgtgcgcttcgacagcgacgtgggggagtaccgggcggtcacggagctcgggcggcccgacgctgagtcctggaaccggcagaaggagctcttggagcagaggcgggccgcggtggacacctactgcagacacaactacggggtgattgagagcttcacggtgcagcggcgag

SEQ ID NO: 192 - DNA, 270 bp, Canis familiaris
cacatttcttggagatgttaaagtccgagtgctatttcaccaacgggacggagcgggtgcggttcgtggaaagatacatccataaccgggaggagcacgtgcgcttcgacagcgacgtgggggagtaccgggcggtcacggagctcgggcggcccgacgctgagtcctggaaccagcagaaggagctcttggagcggaagcgggccgaggtggacacctactgcagacacaactacggggtgattgagagcttcgcggtgcagcggcgag

SEQ ID NO: 193 - DNA, 270 bp, Canis familiaris
cacatttcttggagatgttaaagttcgagtgccatttcaccaacgggacggagcgggtgcggtatctgatgagagacatctataaccgggaggagcacgtgcgcttcgacagcgacgtgggggagttccgggcggtcacggagctcgggcggcccgacgctgagtactggaacgggcagaaggagctcttggagcggaggcgggccgaggtggacacggtgtgcagacacaactacggggtgattgagagcttcacggtgcagcggcgag

SEQ ID NO: 194 - DNA, 270 bp, Canis familiaris
cacatttcgtgtacctgtttaagcccgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaacgggcagaaggagatcttggagcaggagcgggcaacggtggacacctactgcagacacaactacggggtgattgagagcttcacggtgcagcggcgag

SEQ ID NO: 195 - DNA, 270 bp, Canis familiaris
cacatttcttgaagatggtaaagttcgagtgccatttcaccaacgggacggagcgggtgcggttggtggaaagagacatctataaccgggaggagtacgtgcgcttcgacagcgacgtgggggagttccgggcggtcacggagctcgggcggccctcggctgagtcctggaaccggcagaaggagttcttggagcagaggcgggccgaggtggacacggtgtgcagacacaactaccgggtgggcgagagcttcacggtgcagcggcgag

SEQ ID NO: 196 - DNA, 270 bp, Canis familiaris
cacatttcttggagatgttaaagtccgagtgccatttcaccaacgggacggagcgggtgcggttcgtggaaagatacatccataaccgggaggagaacgtgcgcttcgacagcgacgtgggggagtaccgggcggtcacggagctcgggcggcccgacgctgagtcctggaaccggcagaaggagctcttggagcggaagcgggccgaggtggacacctactgcagacacaactacggggtgattgagagcttcacggtgcagcggcgag

SEQ ID NO: 197 - DNA, 270 bp, Canis familiaris
cacatttcttgaagatggtaaagttcgagtgccatttcaccaacgggacggagcgggtgcggttggtggaaagagacatctataaccgggaggagtacgtgcgcttcgacagcgacgtgggggagttccgggcggtcacggagctcgggcggccctcggctgagtactggaacgggcagaaggagctcttggagcagaggcgggccgaggtggacacggtgtgcagacacaactacggggtgggcgagagcttcacggtgcagcggcgag

SEQ ID NO: 198 - DNA, 270 bp, Canis familiaris
cacatttcgtgtacctgtttaagcccgagtgccatttcaccaacgggacggagcgggtgcggttcgtggaaagatacatccataaccgggaggagttcgtgcgcttcgacagcgacgtgggggagtaccgggcggtcacggagctcgggcggcccgtcgctgagtcctggaacgggcagaaggagatcttggagcagaagcgggccgcggtggacacctactgcagacacaactacggggtgattgagagcttcacggtgcagcggcgag

SEQ ID NO: 199 - DNA, 270 bp, Canis familiaris
cacatttcttggaggtggcaaagtccgagtgctatttcaccaacgggacggagcgggtgcggttcgtggaaagatacatccataaccgggaggagaacgtgcgcttcgacagcgacgtgggggagtaccgggcggtcacggagctcgggcggcgcgacgctgagtcctggaaccggcagaaggagctcttggagcggaagcgggccgaggtggacacctactgcagacacaactacggggtgattgagagcttcacggtgcagcggcgag

SEQ ID NO: 200 - DNA, 270 bp, Canis familiaris
cacatttcttgaagatggtaaagttcgagtgccatttcaccaacgggacggagcgggtgcggtatctgatgagagacatctataaccgggaggagttcgtgcgcttcgacagcgacgtgggggagttccgggcggtcacggagctcgggcggcgcgacgctgagtcctggaacgggcagaaggagctcttggagcagaagcgggccgcggtggacacctactgcagacacaactacggggtgattgagagcttcacggtgcagcggcgag

SEQ ID NO: 201 - DNA, 270 bp, Canis familiaris
cacatttcgtgaagatgtttaaggccgagtgccatttcaccaacgggacggagcgggtgcggcttctggcgagaagcatctataaccgggaggagttcgtgcgcttcgacagcgacgtgggggagttccgggcggtcacggagctcgggcggcccgacgctgagtcctggaaccggcagaaggagttcttggagcagaggcgggccgcggtggacacctactgcagacacaactaccgggtgggcgagagcttcacggtgcagcggcgag

SEQ ID NO: 202 - DNA, 270 bp, Canis familiaris
cacatttcgtggaggtgtttaaggccgagtgccatttcaccaacgggacggagcgggtgcggttcgtggaaagaagcatctataaccgggaggagtacgtgcgcttcgacagcgacgtgggggagtaccgggcggtcacggagctcgggcggcccgacgctgagtactggaacccgcagaaggagctcttggagcgggggcgggccgaggtggacacctactgcagacacaactacggggtgggcgagagcttcacggtgcagcggcgag

SEQ ID NO: 203 - DNA, 270 bp, Canis familiaris
cacatttcttggagatgttaaagttcgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaaccggcagaaggagctcttggagcagaggcgggccgaggtggacacctactgcagacacaactacggggtgggcgagagcttcacggtgcagcggcgag

SEQ ID NO: 204 - DNA, 270 bp, Canis familiaris
cacatttcttgaagatggtaaagttcgagtgccatttcaccaacgggacggagcgggtgcggtttgtggaaagatacatctataaccgggaggagtacgtgcgcttcgacagcgacgtgggggagtaccgggcggtcacggagctcgggcggcccgacgctgagtcctggaaccggcagaaggagctcttggagcagaggcgggccgcggtggacacctactgcagacacaactacggggtgggcgagagcttcacggtgcagcggcgag

SEQ ID NO: 205 - DNA, 270 bp, Canis familiaris
cacatttcttgaacgtggcaaagtccgagtgctatttcaccaacgggacggagcgggtgcggttcgtggacagatacatctataaccgggaggagttcgtgcgcttcgacagcgacgtgggggagttccgggcggtcacggagctcgggcggcccgacgctgagtacctgaaccggcagaaggagatcttggagcaggagcgggccgcggtggacacctactgcagacacaactacggggtgggcgagagcttcacggtgcagcggcgag

SEQ ID NO: 206 - DNA, 270 bp, Canis familiaris
cacatttcttgaacgtggcaaagtccgagtgctatttcaccaacgggacggagcgggtgcggttcgtggacagatacatctataaccgggaggagttcgtgcgcttcgacagcgacgtgggggagttccgggcggtcacggagctcgggcggcccgacgctgagtacctgaaccggcagaaggagatcttggagcaggagcgggccgcggtggacacctactgcagacacaactacggggtgattgagagcttcacggtgcagcggcgag

SEQ ID NO: 207 - DNA, 270 bp, Canis familiaris
cacatttcgtgtaccagtttaagggcgagtgctatttcaccaacgggacggagcgggtgcggcttctggcgagaagcatctataaccgggaggagttcgtgcgcttcgacagcgacgtgggggagttccgggcggtcacggagctcgggcggcccgacgctgagtactggaaccggcagaaggagctcttggagcagaggcgggccgcggtggacacctactgcagacacaactacggggtgattgagagcttcacggtgcagcggcgag

SEQ ID NO: 208 - DNA, 270 bp, Canis familiaris
cacatttcttgaacgtggcaaagtccgagtgctatttcaccaacgggacggagcgggtgcggttcgtggacagatacatctataaccgggaggagttcgtgcgcttcgacagcgacgtgggggagttccgggcggtcacggagctcgggcggcccgacgctgagtactggaaccggcagaaggagatcttggagcaggagcgggccgcggtggacacctactgcagacacaactacggggtgattgagagcttcacggtgcagcggcgag

SEQ ID NO: 209 - DNA, 270 bp, Canis familiaris
cacatttcttggaggtggcaaagtycgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaacgggcagaaggagctcttggagcaggagcgggccgcggtggacacctactgcagacacaactaccgggtgggcgagagcttcacggtgcagcggcgag

SEQ ID NO: 210 - DNA, 270 bp, Canis familiaris
cacatttcttggagatgttaaagttcgagtgccatttcaccaacgggacggagcgggtgcggtatctggtgagagacatctataaccgggaggagcacgtgcgcttcgacagcgacgtgggggagttccgggcggtcacggagctcgggcggcccgacgctgagtactggaacgggcagaaggagctcttggagcagaagcgggccgcggtggacacctactgcagacacaactacggggtgattgagagcttcgcggtgcagcggcgag

SEQ ID NO: 211 - DNA, 270 bp, Canis familiaris
cacatttcttggaggtggcaaaggycgagtgccatttcaccaacgggacggagcgggtgcggttcgtggaaagatacatctataaccgggaggagtacgtgcgcttcgacagcgacgtgggggagtaccgggcggtcacggagctcgggcggcccgacgctgagtcctggaaccggcagaaggagatcttggagcaggagcgggcaacggtggacacggtgtgcagacacaactacggggtgggcgagagcttcacggtgcagcggcgag

SEQ ID NO: 212 - DNA, 270 bp, Canis familiaris
cacatttcttgaagatggtaaagttcgagtgccatttcaccaacgggacggagcgggtgcggcttctggtgagagacatctataaccgggaggagcacgtgcgcttcgacagcgacgtgggggagtaccgggcggtcacggagctcgggcggcccgacgctgagtactggaacgggcagaaggagctcttggagcagaggcgggccgaggtggacacggtgtgcagacacaactacggggtgattgagagcttcacggtgcagcggcgag

SEQ ID NO: 213 - DNA, 270 bp, Canis familiaris
cacatttcttggaggtggcaaagtccgagtgccatttcaccaacgggacggagcgggtgcggttcgtggaaagatacatccataaccgggaggagttcgtgcgcttcgacagcgacgtgggggagtaccgggcggtcacggagctcgggcggcccggcgctgagtcctggaaccggcagaaggagctcttggagcggaagcgggccgaggtggacacggtgtgcagacacaactacggggtgattgagagcttcacggtgcagcggcgag

SEQ ID NO: 214 - DNA, 270 bp, Canis familiaris
cacatttcttggagatgttaaagtccgagtgctatttcaccaacgggacggagcgggtgcggttcgtggaaagatacatccataaccgggaggagcacgtgcgcttcgacagcgacgtgggggagtaccgggcggtcacggagctcgggcggcccgacgctgagtcctggaaccggcagaaggagctcttggagcggaagcgggccgaggtggacacctactgcagacacaactacggggtgattgagagcttcgcggtgcagcggcgag

SEQ ID NO: 215 - DNA, 270 bp, Canis familiaris
cacatttcttggagatggtaaagttcgagtgccatttcaccaacgggacggagcgggtgcggtatctggaaagatacatctataaccgggaggagatcgtgcgcttcgacagcgacgtgggggagtaccgggcggtcacggagctcgggcggcccatcgctgagtcctggaaccggcagaaggagctcttggagcagaggcgggccgcggtggacacctactgcagacacaactacggggtgattgagagcttcacggtgcagcggcgag

SEQ ID NO: 216 - DNA, 270 bp, Canis familiaris
cacatttcttggagatgttaaagttcgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaaccggcagaaggagctcttggagcaggagcgggccgcggtggacacggtgtgcagacacaactaccgggtgggcgagagcttcacggtgcagcggcgag

SEQ ID NO: 217 - DNA, 270 bp, Canis familiaris
cacatttcttggagatggtaaagttcgagtgccatttcaccaacgggacggagcgggtgcggcttctggtgagagacatctataaccgggaggagcacgtgcgcttcgacagcgacgtgggggagttccgggcggtcacggagctcgggcggcccgacgctgagtactggaacgggcagaaggagatcttggagcaggagcgggcaacggtggacacctactgcagacacaactacggggtgattgagagcttcacggtgcagcggcgag

SEQ ID NO: 218 - DNA, 270 bp, Canis familiaris
cacatttcgtgaagatgtttaaggccgagtgccatttcaccaacgggacggagcgggtgcggcttctggcgagagacatctataaccgggaggagttcgtgcgcttcgacagcgacgtgggggagtaccgggcggtcacggagctcgggcggcgcgacgctgagtcctggaaccggcagaaggagctcttggagcgggcgcgggccgcggtggacacctactgcagacacaactaccgggtgggcgagagcttcacggtgcagcggcgag

SEQ ID NO: 219 - DNA, 270 bp, Canis familiaris
cacatttcttggagatggtaaagttcgagtgccatttcaccaacgggacggagcgggtgcggtatgtgctgagagacatctataaccgggaggagatcgtgcgcttcgacagcgacgtgggggagttccgggcggtcacggagctcgggcggcccgacgctgagtcctggaaccggcagaaggagctcttggagcagaagcgggccgcggtggacacctactgcagacacaactaccgggtgggcgagagcttcacggtgcagcggcgag

SEQ ID NO: 220 - DNA, 270 bp, Canis familiaris
cacatttcgtgtaccagtttaagcccgagtgccatttcaccaacgggacggagcgggtgcggttcgtggaaagatacatccataaccgggaggagttcgtgcgcttcgacagcgacgtgggggagtaccgggcggtcacggagctcgggcggcccgtcgctgagtcctggaacgggcagaaggagctcttggagcaggagcgggccgcggtggacacctactgcagacacaactacggggtgattgagagcttcacggtgcagcggcgag

SEQ ID NO: 221 - DNA, 270 bp, Canis familiaris
cacatttcgtgaagatggctaaggccgagtgccatttcaccaacgggacggagcgggtgcggtttctggcaagaaacatctataaccgggaggagttcgtgcgcttcgacagcgacgtgggggagttccgggcggtcacggagctcgggcggcccgacgctgagtcctggaaccggcagaaggagctcttggagcgggagcgggccgcggtggacacctactgcagacacaactaccgggtgggcgagagcttcacggtgcagcggcgag

SEQ ID NO: 222 - DNA, 270 bp, Canis familiaris
cacatttcttgaagatggtaaagtccgagtgctatttcaccaacgggacggagcgggtgcggttcgtggaaagatacatccataaccgggaggagctcgtgcgcttcgacagcgacgtgggggagttccgggcggtcacggagctcgggcggcccgacgctgagtcctggaaccggcagaaggagctcttggagcggaagcgggccgaggtggacacggtgtgcagacacaactacggggtgattgagagcttcacggtgcagcggcgag

SEQ ID NO: 223 - DNA, 270 bp, Canis familiaris
cacatttcgtgaagatgtttaagtccgagtgccatttcaccaacgggacggagcgggtgcggtatctggcgagagacatctataaccgggaggagttcgtgcgcttcgacagcgacgtgggggagttccgggcggtcacggagctcgggcggcccgacgctgagtcctggaaccggcagaaggagctcttggagcgggcgcgggccgcggtggacacctactgcagacacaactaccgggtgggcgagagcttcacggtgcagcggcgag

SEQ ID NO: 224 - DNA, 270 bp, Canis familiaris
cacatttcttggagatgttaaaggccgagtgccatttcaccaacgggacggagcgggtgcggtatctggtgagagacatctataaccgggaggagttggtgcgcttcgacagcgacgtgggggagttccgggcggtcacggagctcgggcggcccgacgctgagtactggaacgggcagaaggagatcttggagcggaggcgggccgagctggacacggtgtgcagacacaactacggggtgattgagagcttcacggtgcagcggcgag

SEQ ID NO: 225 - DNA, 270 bp, Canis familiaris
cacatttcttggagatgttaaagttcgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaaccggcagaaggagttcttggagcagaggcgggccgaggtggacacctactgcagacacaactacggggtgggcgagagcttcacggtgcagcggcgag

SEQ ID NO: 226 - DNA, 270 bp, Canis familiaris
cacatttcttgaagatgtcaaagtccgagtgctatttcaccaacgggacggagcgggtgcggttggtggaaagatgcatctataaccgggaggagtacgtgcgcttcgacagcgacgtgggggagtaccgggcggtcacggagctcgggcggccctcggctgagtcctggaacgggcagaaggagttcttggagcagaagcgggccgaggtggacacggtgtgcagacacaactacggggtgggcgagagcttcacggtgcagcggcgag

SEQ ID NO: 227 - DNA, 270 bp, Canis familiaris
cacatttcttggagatgttaaagtccgagtgctatttcaccaacgggacggagcgggtgcggttcgtggaaagatacatccataaccgggaggagcacgtgcgcttcgacagcgacgtgggggagtaccgggcggtcacggagctcgggcggcccgacgctgagtcctggaaccggcagaaggagctcttggagcggaagcgggccgaggtggacacctactgcagacacaactacggggtgattgagagcttcacggtgcagcggcgag

SEQ ID NO: 228 - DNA, 270 bp, Canis familiaris
cacatttcttggaggtggcaaagtccgagtgctatttcaccaacgggacggagcgggtgcggttcgtggaaagatacatctataaccgggaggagtacgtgcgcttcgacagcgacgtgggggagtaccgggcggtcacggagctcgggcggcgcgacgctgagtcctggaaccggcagaaggagctcttggagcggaagcgggccgcggtggacacctactgcagacacaactaccgggtgggcgagagcttcacggtgcagcggcgag

SEQ ID NO: 229 - DNA, 270 bp, Canis familiaris
cacatttcttggagatggtaaagtccgagtgctatttcaccaacgggacggagcgggtgcggttcgtggaaagatacatccataaccgggaggagctcgtgcgcttcgacagcgacgtgggggagtaccgggcggtcacggagctcgggcggcccgacgctgagtcctggaaccggcagaaggagctcttggagcggaagcgggccgaggtggacacggtgtgcagacacaactacggggtgattgagagcttcacggtgcagcggcgag

SEQ ID NO: 230 - DNA, 270 bp, Canis familiaris
cacatttcgtgaggatgtataaggccgagtgccatttcaccaacgggacggagcgggtgcggtatctgatgagagacatctataaccgggaggagttcgcgcgcttcgacagcgacgtgggggagtaccgggcggtcacggagctcgggcggcgcgacgctgagtcctggaaccggcagaaggagctcttggagcggaggcgggccgaggtggacacctactgcagacacaactacggggtgattgagagcttcacggtgcagcggcgag

SEQ ID NO: 231 - DNA, 270 bp, Canis familiaris
cacatttcttggaggtggcaaagtccgagtgctatttcaccaacgggacggagcgggtgcggttcgtggaaagatacatccataaccgggaggagttcgtgcgcttcgacagcgacgtgggggagtaccgggcggtcacggagctcgggcggccctcggctgagtcctggaacgggcagaaggagatcttggagcaggagcgggcaacggtggacacctactgcagacacaactacggggtgattgagagcttcacggtgcagcggcgag

SEQ ID NO: 232 - DNA, 270 bp, Canis familiaris
cacatttcttgaagatggtaaagttcgagtgccatttcaccaacgggacggagcgggtgcggttggtggaaagagacatctataaccgggaggagtacgtgcgcttcgacagcgacgtgggggagtaccgggcggtcacggagctcgggcggccctcggctgagtcctggaaccggcagaaggagttcttggagcagaggcgggccgcggtggacacctactgcagacacaactacggggtgggcgagagcttcacggtgcagcggcgag

SEQ ID NO: 233 - DNA, 270 bp, Canis familiaris
cacatttcgtgtaccagtttaagggcgagtgccatttcaccaacgggacggagcgggtgcggtttctggcgagaagcatctataaccgggaggagttcgtgcgcttcgacagcgacgtgggggagtaccgggcggtcacggagctcgggcggcccatcgctgagtcctggaaccggcagaaggagttcttggagcggaggcgggccgaggtggacacctactgcagacacaactacggggtgattgagagcttcacggtgcagcggcgag

SEQ ID NO: 234 - DNA, 270 bp, Canis familiaris
cacatttcttggaggtggcaaagtccgagtgctatttcaccaacgggacggagcgggtgcggttcgtggaaagatacatccataaccgggaggagaacgtgcgcttcgacagcgacgtgggggagttccgggcggtcacggagctcgggcggcccgacgctgagtactggaacgggcagaaggagctcttggagcagaagcgggccgcggtggacacctactgcagacacaactacggggtgggcgagagcttcacggtgcagcggcgag

SEQ ID NO: 235 - DNA, 270 bp, Canis familiaris
cacatttcgtgaggatgtttaaggccgagtgctatttcaccaacgggacggagcgggtgcggttggtggaaagagacatctataaccgggaggagttcgtgcgcttcgacagcgacgtgggggagtaccgggcggtcacggagctcgggcggcccgacgctgagtactggaacgggcagaaggagctcttggagcagaggcgggccgaggtggacacctactgcagacacaactaccgggtgggcgagagcttcacggtgcagcggcgag

SEQ ID NO: 236 - DNA, 270 bp, Canis familiaris
cacatttcgtgaggatgtttaaggccgagtgctatttcaccaacgggacggagcgggtgcggttggtggaaagagacatctataaccgggaggagtacgtgcgcttcgacagcgacgtgggggagtaccgggcggtcacggagctcgggcggcccatcgctgagtcctggaaccggcagaaggagttcttggagcagaggcgggccgcggtggacacctactgcagacacaactaccgggtgggcgagagcttcacggtgcagcggcgag

SEQ ID NO: 237 - DNA, 270 bp, Canis familiaris
cacatttcttggagatggtaaagttcgagtgccatttcaccaacgggacggagcgggtgcggtatctggcgagagacatctataaccgggaggagatcctgcgcttcgacagcgacgtgggggagttccgggcggtcacggagctcgggcggcccatcgctgagtcctggaaccggcagaaggagatcttggagcagaggcgggccgcggtggacacctactgcagacacaactacggggtgattgagagcttcgcggtgcagcggcgag

SEQ ID NO: 238 - DNA, 19 bp, Artificial Sequence
  Primer
gatccccccgtccccacag

SEQ ID NO: 239 - DNA, 15 bp, Artificial Sequence
  Primer
cgcccgctgcgctca

SEQ ID NO: 240 - DNA, 20 bp, Artificial Sequence
  Primer
taaggttcttttctccctct

SEQ ID NO: 241 - DNA, 20 bp, Artificial Sequence
  Primer
ggacagattcagtgaagaga

SEQ ID NO: 242 - DNA, 20 bp, Artificial Sequence
  Primer
ctcactggcccggctgtctc

SEQ ID NO: 243 - DNA, 20 bp, Artificial Sequence
  Primer
cacctcgccgctgcaacgtg